(19) United States
(12) Patent Application Publication
(10) Pub. No.: US 2010/0169988 A1
(43) Pub. Date: Jul. 1, 2010

(54) MEANS AND METHODS FOR ISOLATING AND DETERMINING NOVEL TARGETS FOR THE TREATMENT OF NEURODEGENERATIVE, NEUROLOGICAL OR NEUROPSYCHIATRIC DISORDERS AND COMPOSITIONS COMPRISING THE SAME

(76) Inventors: Bernhard Kohli, Zurich (CH); Uwe Konietzko, Zurich (CH); Roger Nitsch, Zumikon (CH); Jan Grimm, Dubendorf (CH)

Correspondence Address: LATIMER INTELLECTUAL PROPERTY LAW, LLP, P.O. BOX 711200, HERNDON, VA, 20171, US

(21) Appl. No.: 12/517,420

(22) PCT Filed: Dec. 6, 2007
(86) PCT No.: PCT/EP2007/010631

§ 371 Date: Dec. 8, 2009

(30) Foreign Application Priority Data
Dec. 6, 2006 (EP) 06025239.2

(51) Int. Cl. G01N 33/00; A61K 38/43; A61K 38/45; A61K 38/46; C12Q 1/68; G01N 33/53; G01N 33/573; C12N 9/12; C12N 9/14; C12N 9/90; C12N 5/00; C40B 40/06; A61K 38/16; C07K 14/00; A01K 67/00; A61K 38/52

(52) U.S. Cl. 800/3; 424/94.1; 424/94.5; 424/94.6; 435/6; 435/7.1; 435/7.4; 435/194; 435/195; 435/233; 435/325; 506/16; 514/12; 530/350; 800/9

(57) ABSTRACT

A novel method of identifying and obtaining molecules interacting with neurodegenerative, neurological or neuropsychiatric disorder-associated proteins is provided, which is suitable for drug screening and drug development. Furthermore, drugs and drug targets for the therapeutic intervention of neurodegenerative, neurological or neuropsychiatric disorders, in particular Alzheimer's disease are described.

### FIELD OF THE INVENTION

The present invention generally relates to the technical field of medicine, in particular to the field of neurodegenerative, neurological or neuropsychiatric disorders. More specifically, the invention relates to methods of identifying and obtaining target proteins involved in neurodegenerative, neurological or neuropsychiatric disorders and neurodegenerative diseases in particular. The present invention further concerns the use of those target proteins in methods of screening and isolating therapeutic agents for treating neurodegenerative, neurological or neuropsychiatric disorders, in particular Alzheimer's disease (AD). In a further aspect, the present invention relates to an animal model useful in screening, isolating and testing of compounds and therapeutic agents. Furthermore, an in-vivo assay is provided for testing and validating compounds, compositions and agents for their potential efficacy as therapeutics for the treatment of neurodegenerative, neurological or neuropsychiatric disorders, in particular AD and other amyloidoses.

### BACKGROUND OF THE INVENTION

Alzheimer's disease (AD) is a chronic progressive neurodegenerative disease which is clinically characterized by progressive deficits in memory leading to complete erosion of higher cognitive functions. The pathology of Alzheimer's disease is characterized by three major hallmarks: β-amyloid plaques and vascular β-amyloid composed of proteinacious deposits of β-amyloid peptide; neurofibrillary tangles composed of tau-protein; and Lewy bodies composed of α-synuclein. Because most current evidence points to an important role of β-amyloid (An) peptide aggregates in the primary cause of neurodegeneration in Alzheimer's disease, there is a major focus in the research on Aβ peptide formation, aggregation, and turnover in order to identify targets for the development of drugs designed to reduce its formation or to activate mechanisms that accelerate its clearance from brain. An overview concerning Alzheimer dementia, neuropathological changes in the disease state and genetic causes as well as other risk factors is given below in the detailed description of the invention.

The amyloid precursor protein (APP) is a widely expressed transmembrane protein that is the source of the β-amyloid peptide and other peptide fragments with varying effects on neural function (Mattson, Nature 430 (2004), 631-639). Cleavage of APP by beta secretase releases the soluble ectodomain of APP (sAPPbeta) and generates a membrane bound C-terminal fragment (C99), which is then cleaved by gamma secretase to release the β-amyloid peptide and the APP intracellular domain that can translocate to the nucleus and regulate gene expression. An alternative cleavage starting with alpha-secretase is non-amyloidogenic and releases secreted APPalpha, which has neuroprotective effects and regulates cell excitability and synaptic plasticity. While it is commonly accepted that Abeta peptides are a key factor in Alzheimer's disease, a direct role for APP in its full-length configuration cannot be excluded. The precise biological functions of APP are not fully understood, but increasing evidence suggests it has important roles in regulating neuronal survival, neurite outgrowth, synaptic plasticity, vesicle transport, trafficking of growth factors and cell adhesion among others.

Hence, there is still a need for therapeutic and diagnostic means for the treatment of AD as well as other disorders associated with APP.

### SUMMARY OF THE INVENTION

Assays described in the prior art mainly target the formation of Aβ by blocking the secretases (γ or β) responsible for cleaving the amyloid precursor protein (APP). In contrast, the method of the present invention uses APP for identifying molecules, in particular proteins which are associated with APP function, including APP processing, cellular trafficking, degradation, isomerization, modification and direct or indirect regulation by APP of downstream processes like neuronal survival, synaptic plasticity, trafficking of growth factors, glucose metabolism, among others, and which are thus believed to provide novel targets for therapeutic intervention. In this context, a novel approach for identifying and obtaining molecules which interact with proteins associated with a neurodegenerative, neurological or neuropsychiatric disorder such as AD has been developed. In particular, the present invention provides a system in which a given protein known to be involved in the onset or progression of a neurodegenerative, neurological or neuropsychiatric disorder is fused to a tag and provided within a cellular or physiological tissue environment resembling the corresponding tissue affected by the disorder in a subject, particularly human. More specifically, a non-human transgenic animal is provided, that has been genetically engineered to express recombinant, tagged APP as “bait” in the brain in order to identify and isolate brain molecules that interact with APP either directly or indirectly, via other binding molecules. Samples taken from brain tissue, cells or fluid can directly be used for the purification of APP complexes and subsequent analysis including isolating and determining APP interacting molecules. The system of the present invention has the advantage that it allows for the direct purification of binding partners from cells or tissues, and overcomes the drawbacks of in vitro methods performed in artificial, un-physiologic, environments that influence the interaction of APP with its binding partners.

Besides providing a native, and physiologically relevant, environment for complex formation of the tagged protein with its putative binding partners, a further advantage of the system of the present invention is that specifically interacting proteins can be identified and isolated, that are present, induced or more abundant, respectively, under pathological conditions as compared to healthy conditions. One such pathological condition could be generated by crossing the “tagged” mice with mouse models of diseases. More specifically, these models could be models of Alzheimer's disease, i.e. mice that express in brain amyloid, tau or α-synuclein pathologies. This substantially reduces the risk of false positive results as compared to conventional assays in which the target protein is subjected to samples of proteins or fragments thereof under unbiased conditions, and molecules may be identified that do not bind to APP, for example under native pathological conditions. Accordingly, it is prudent to expect that proteins identified in accordance with the method of the present invention to bind to the tagged protein involved in the neurodegenerative, neurological or neuropsychiatric disorder and agents capable of modulating the so identified proteins are indeed associated with the onset or progression of the disorder as well and therefore provide suitable targets for therapeutic intervention and are useful as diagnostic markers. Thus, the present invention also relates to a method for treating a neurodegenerative, neurological or neuropsychiatric disorder in a subject comprising administration to the subject an agent, wherein said agent is specific for a protein selected from the group consisting of the proteins referred to in tables 1, 2, 4, 5, 13 and 14, infra, and the corresponding human orthologs, paralogs or homologs thereof or is derived from such protein and binds to APP. Preferably, such binding results in the inhibition of functions or processing patterns that contribute to central nervous system disease, including amyloidogenic APP processing, cellular trafficking, signaling, degradation, isomerization, modification and direct or indirect regulation by APP of downstream processes like neuronal survival, synaptic plasticity, trafficking of growth factors, glucose metabolism, and most preferably said agent can cross the blood brain carrier.

While in the following the present invention will be explained in more detail with respect to APP and AD, it is to be understood that unless indicated otherwise, the embodiments disclosed herein are equally applicable to any other proteins associated with a neurodegenerative, neurological or neuropsychiatric disorder, in particular with respect to those identified in the experimental section, infra.

In the following, reference is made to previous European patent application EP 06 025 239.2, filed Dec. 6, 2006 and titled “Means and methods for isolating and determining novel targets for the treatment of neurodegenerative, neurological or neuropsychiatric disorders and compositions comprising the same”, a copy of which is contained with the international file.

### BRIEF DESCRIPTIONS OF THE DRAWINGS

FIG. 1 (corresponding to FIG. 1-1 of EP 06 025 239.2): describes APP processing: APP processing can follow two fundamentally different pathways, the so-called non-amyloidogenic and the amyloidogenic pathway, depending on whether α- or β-secretase is responsible for ectodomain shedding, which is a prerequisite for cleavage by γ-secretase, which in a two-step cleavage at the γ-41 or 42 and then the S₃ cleavage site of APP releases the intracellular domain (AICD). The subcellular localization of these individual cleavage steps and factors influencing the predominance of the α- vs. the β-cleavage pathway are discussed in the following subchapters. Lumenal or extracellular Aβ is believed to first form oligomeric, soluble aggregates, before forming high molecular weight fibrillar aggregates that are believed to consist of tetrameric Aβ aggregates stacked on top of each other in 15° step shifts, labeled “fibrillar Aβ” in the upper right of the figure (Li et al. 1999). Cleavage steps and positions are denoted by the corresponding Greek symbol and the legend at the bottom right describes the icons used throughout the figure.

FIG. 2 (corresponding to FIG. 1-2 of EP 06 025 239.2): schematically represents the amyloid cascade hypothesis: A large body of evidence shows Aβ pathology to precipitate Tau pathology and to be the main trigger for AD pathology and symptoms. New data testifying to this is summarized from (Oddo et al. 2003) in the graph at the bottom left: Active immunization (“injection”) of TauP301L, swAPP, PS1 (M146V) triple transgenic mice results in rapid plaque clearance and belated reduction of staining for hyperphosphorylated Tau, with a similar time lag occurring during redevelopment of Aβ and Tau deposits. Schematic: This figure focuses on the amyloidogenic processing pathway of APP (cleavage sites at top), with the general processing and topology of APP shown in FIG. 1. The central toxic species in terms of aggregation level of Aβ is still a matter of debate—probably different forms of Aβ, also depending on intracellular or extracellular presence, result in different cellular and immune system responses. It is thought that Aβ pathology disturbs the equilibrium between Tau phosphatases and kinases, resulting in higher levels and phosphorylation status (P) of Tau, with the resulting aggregation and formation of paired helical filaments depicted as grey and white strands. Both Aβ and Tau pathology finally are responsible for the ensemble of symptoms that make up AD.

FIG. 3 (corresponding to FIG. 1-3 of EP 06 025 239.2): shows that APP is a single pass trans-membrane protein containing several functional domains: The large extracellular portion of the protein (left) contains a leader signal peptide (PepSig) that is rapidly cleaved after correct targeting to and insertion into the ER membrane, Heparin-, Copper-, Zinc-, Collagen-binding and Chondroitin sulfate attachment regions (Hep, Cu, Zn, Col and ChonS, respectively) that are important for interaction with the ECM, the Kunitz Protease Inhibitor domain, all as described above, and finally, the infamous Aβ-region strongly implied in the pathophysiology of AD. The intracellular domain (right) contains two main binding regions for cytosolic proteins: a G0-protein binding juxtamembrane region potentially implied in any putative receptor function of APP as well as the YENPTY region that has been shown to bind to a network of proteins involved in nuclear signaling, cytoskeleton adhesion and vesicle transport, among other functions.

FIG. 4 (corresponding to FIG. 1-4 of EP 06 025 239.2): schematically shows the major players in the AICD protein interaction network and their roles: The intracellular domain of APP is shown horizontally, including its sequence and the important YENPTY domain. Possible sites of phosphorylation are denoted by “P”. Interactors are grouped by dotted lines according to functional pathways, which are discussed throughout the detailed description of the invention. Endocytosis is important for intracellular trafficking of APP and secretase access and is mediated by Adaptin/Clathrin coating of vesicles, while Dynamin is responsible for pinching them off from the membrane. X11 is a processing modifier of APP, putatively through its additional interaction with Presenilins (PS). Like X11, the mDab protein, which is involved in neuronal development, also has a PTB domain with which it can interact with AICD. Abl Kinase and Jun-N-terminal Kinase (JNK) can phosphorylate AICD at Y683 and T668, respectively, which is one mode of regulating binding to AICD. JNK binds to AICD via JNK-interacting protein (MP), thus linking AICD to the cellular stress response pathway. Indirect interaction of AICD with the cytoskeleton is severalfold: Fe65, a major direct interactor of AICD, can bind to the N-terminal domain of Tau protein, linking it to microtubules and enabling vesicular transport. Also, Fe65 binds Mena through its N-terminal WW-domain, linking AICD to the Actin cytoskeleton through Menas interaction with Profilin. Importantly, Fe65 is also a crucial player in the nuclear signaling pathway of AICD on which we focus: it shuttles AICD to the nucleus and can additionally interact with Tip60 to form a transcriptionally active nuclear complex. Some cross-interactions between pathways or additional transmembrane proteins such as Presenilins and Low Density Lipoprotein Receptor (LRP) are left out for simplicity. Important protein subdomains are JBD (JNK binding domain), KB (Kinesin light chain binding domain), WW (WW-domain) and the PTBs already mentioned.

FIG. 5 (corresponding to FIG. 1-6 of EP 06 025 239.2): schematically shows the LC-MS/MS workflow: 1) A solution of proteins, e.g. from an affinity purification, is digested using the amino-acid specific protease Trypsin, which cleaves exclusively after Lysine or Arginine residues, except when directly followed by a Proline. 2) The desalted peptide digest is injected into a reverse phase capillary that 3) separates peptides according to hydrophobicity in a positive gradient of volatile organic solvent such as Acetonitrile. 4) The first MS scan separates peptides according to m/z ratio and the data-dependent machine control software chooses the strongest signals for fragmentation 5), resulting in collision-induced dissociation into 6) b- and y-ion series that are analyzed in a second MS scan. 7) Protein sequences in a protein database are cleaved in silico by Trypsin and the theoretical masses of b- and y-series ions from the resulting peptides are calculated and 8) correlated with the experimental spectra. 9) Good fits of experimental and theoretical spectra allow identification of protein components in the original mixture.

FIG. 6 (corresponding to FIG. 3-5 of EP 06 025 239.2): shows that the specific release of AICD peptides with bound interactors by PreScission cleavage reduces contaminant background proteins: A: Schematic of the PreScission protease cleavage procedure; grey boxes=contaminant proteins unspecifically bound to the matrix, circles shaded grey=the desired AICD interacting proteins, triple colored bar (grey-black-white)=Biotin/PreScission recognition site/N-terminal AICD peptide, scissors=PreScission protease. B: Undifferentiated SH-SY5Y cells were lysed and bound to magnetic beads coated with Streptavidin-bound synthetic PrSciAICD(wt)/(mut). Two conventional wash steps were followed by a wash step with PreScission buffer and cleavage mediated peptide release from the matrix by addition of PreScission protease. Legend: wt=PrSciAICD(wt) eluted with PreScission protease, LDS=remaining matrix from the cleavage step eluted with LDS gel loading buffer, mut=PrSciAICD(mut) eluted with PreScission protease. It is evident that the ratio of eluted X11α to total protein in the wt lane is far higher than that from the LDS lane, which shows that the desired background reducing effect was precisely obtained.

FIG. 7 (corresponding to FIG. 3-8 of EP 06 025 239.2): shows that conventional dynamic exclusion algorithms do not take chromatographic peak information into account, in contrast to Fulspec: This schematic depicts differences between currently employed exclusion rules and the idea behind Fulspec. It represents a slice through the data from a typical LC-MS/MS run with the m/z value remaining constant. The first broad peak probably corresponds to an excessively abundant peptide. Based on signal intensity, both algorithms might choose the first CID. Conventional dynamic exclusion repeats sampling this same peptide, not yielding new information, until the predefined threshold number of CIDs from the same m/z value is achieved. This is followed by a rigid exclusion period, during which the following two peaks shaded grey would be missed in this case. The Fulspec algorithm takes chromatographic fundamentals into account: Peak to Trough ratio, NewPeak to Trough ratio. Also, the signal increase of a chromatographic peak in relation to the signal intensity at the time of first sampling thereof is a parameter. The exact implementation of the parameters depicted here is discussed in the context of Fulspec rules. In summary, Fulspec would not waste analytical capacity on the “surplus” CIDs and might additionally, at the later timepoints (shaded grey), pick the wanted CIDs.

FIG. 8 (corresponding to FIG. 3-11 of EP 06 025 239.2): represents the TAP-AICD tandem affinity purification established from stably transfected Hek 293 and SH-SY5Y cells: A: The TAP procedure is shown schematically. B: TAP-tagged AICD and empty TAP tag vector alone were both transfected into Hek 293 and SH-SY5Y cells. Both proteins were under control of the strong CMV promoter. Negative selection was applied by treating cells with G418, a Neomycin analog. (Neo: Neomycin resistance cassette). The exact percentage of cells producing TAP-AICD or TAP could not be assessed because of the unreliability of a commercial anti-CBP antibody. Therefore, expression of TAP-AICD was assessed in the first eluate (EL1) from the TAP procedure, using an antibody against the C-terminus of APP (α-CT). CMV: Cytomegalovirus promoter, CBP: Calmodulin Binding Peptide, SBP: Streptavidin Binding Peptide, N=TAP tag alone, A=TAP-AICD, wt=wildtype cells. C: WB of fractions from the entire purification procedure, color coded according to the frames in A. Hek lysate containing TAP-AICD (A) or TAP (N) was applied to Streptavidin sepharose, washed, eluted competitively with Biotin, applied to Calmodulin Sepharose and eluted by administering EGTA. As can be seen in the second-last lane, elution of TAP-AICD by LDS loading buffer demonstrates that application of EGTA alone does not elute all bound TAP-AICD. SN1: supernatant after the Streptavidin binding step, SN2: supernatant after binding to the Calmodulin beads, EL2: second eluate after addition of EGTA. D: As a positive control that the TAP lysis and washing buffers are compatible with the co-pull-down of a known interaction partner, Fe65, Hek 293 cells were cotransfected with both HA-Fe65 and either APP-SBP, or APP-2Myc, lysed and purified using Streptavidin beads. Streptavidin sepharose clearly specifically pulled down Fe65 only where APP-SBP was present. lys=lysate, SN=supernatant, EL=eluate

FIG. 9 (corresponding to FIG. 3-15 of EP 06 025 239.2): shows the pUKBK vector system consists of a small, modular eukaryotic expression system with several different tagged derivatives: A: The basic vector, which is only 3.54 kb, was constructed from the individual elements depicted in boxes (legend in middle). It is shown with APP-Citrine, as inserted through the cassette-swapping system using the restriction sites Sfi I, Asc I, Pme I. Sfi I is a 12 by cutter, and Asc I and Pme I are both restriction enzymes with an 8 by recognition sequence, resulting in a low probability that these restriction sites are present in the cDNA or tag that is to be inserted. Still, if required, staggered PCR can of course be used instead of normal PCR and restriction to generate the necessary inserts. In this case, APP constitutes the cDNA and Citrine the tag. B: The basic system was extended with a variety of tags for observing the subcellular localization in fluorescence microscopy, WB staining or one-step affinity purification. Each cDNA cloned and analyzed can be inserted into any of these five different basic vectors in one cloning step, as the cDNA insert preparation is the same for all. For use in primary neurons, the system was driven by a GAPDH promoter, which confers a more physiological level of expression, but mostly, the stronger CMV promoter is used to advantage in biochemical or microscopy experiments. A brief description of the construction of the system is shown in FIG. 15.

FIG. 10 (corresponding to FIG. 3-20 of EP 06 025 239.2): shows α-CTF and β-CTF constructs and comparison of the Signal Peptide properties compared to APP: A: Peptide Signal of the CTF constructs have properties very similar to that of native APP, in spite of the wholly new context, according to PrediSi (Hiller et al. 2004). For cloning reasons, the CTF constructs have an additional Glycine in second position after Methionine, shifting the cleavage position by one aa. Vertical line denotes cleavage position in the constructs. B: Schematic of constructs, not drawn to scale. Promoter: Cytomegalovirus (CMV).

FIG. 11 (corresponding to FIG. 4-1 of EP 06 025 239.2): schematically represents known and novel putative APP trafficking routes and interactions in the synapse: This schematic combines several of the sorting routes and interactions discussed in the detailed description of the invention, or as indicated by appropriate reference. A: Close up of a synapse. APP is translocated to the synapse in vesicles transported along microtubules by Kinesin (Ferreira et al. 1993). In preparation of Calcium-influx-induced excocytosis, APP may play an as yet undefined role in formation of SNARE complexes, as based on co-purification of all major SNARE complex components with APP from mouse brain. With the presence of 14-3-3 η as an enriched component of APP-TAP-AICD purifications that is functionally hitherto unaccounted for, it might be possibly involved as a scaffolding protein in this assembly. However, clear indications of association of APP with Clathrin and Dynamin have been found, which mediate endocytosis. If α-secretase has not yet performed ecto-domain shedding at the plasma membrane, APP can be cleaved endosomally by BACE1 and γ-secretase. B: While Aβ forms intracellular aggregates or can be secreted, AICD can be shuttled by Fe65 and 14-3-3 γ to the nucleus, where it forms ternary complexes with Tip60 and activates transcription. All proteins that were identified in some form as associated with APP/AICD in our MS experiments have grey font in the legend.

FIG. 12 (corresponding to FIG. 2-1 of EP 06 025 239.2): represents that staggered PCR cloning allows insertion of inserts containing internal restriction sites required for ligation.

FIG. 13 (corresponding to FIG. 2-2 of EP 06 025 239.2): demonstrates that assigning absolute probability values to peptide IDs is based on fitting the score distribution into two distinct populations: The underlying assumption is that quality scores are normally distributed for both incorrect and correct identifications. This concept can also be applied to probability values for entire proteins.

FIG. 14 (corresponding to FIG. 7-1 of EP 06 025 239.2): represents the cloning of the construct for the APP-TAP-AICD transgenic mouse: The TAP cassette was amplified by staggered end PCR from the commercial pN-TAP a vector from Stratagene, inserted via BsrG I and Nco I into an APP construct where Nco I and BsrG I restriction sites had been entered by site directed mutagenesis at the positions indicated in Example 7. The receiving vector already contained another IP—cassette, consisting of Flag, HA and Myc epitopes, which we tested but abandoned in favor of the TAP system. From this plasmid, a second staggered PCR was required to enter the entire APP-TAP-AICD construct into the required Xho I site in a Prion-Promoter containing plasmid. The insert-containing region was fully sequenced prior to vector linearization, purification and microinjection.

FIG. 15 (corresponding to FIG. 7-2 of EP 06 025 239.2): schematically represents the construction of pUKBK-C basic vector: 1) Combination of a custom multiple cloning site and a minimal resistance and replication cassette from a bacterial vector 2) Entry of PGK eukaryotic promoter for Neomycin-resistance conferring protein in eukaryotes for stable selection 3) insertion of GAPDH eukaryotic vector to drive expression of cDNA inserted into the multiple cloning site 4) insertion of a second multiple cloning site with the long and thus rare Sfi I and Pme I restriction sites, with spacer sequences in between 5) replacement of the GAPDH promoter with the stronger viral CMV promoter 6) insertion of a polyadenylation signal (poly A) at the 3′ end of the Sfi-Pme flanked cDNA insertion region. Small “c” denotes oligonucleotide ends that are only compatible with the sticky end overhang of the respective enzyme but do not reconstitute the correct restriction sequence on ligation, effectively eliminating one occurrence of the restriction site.

### ABBREVIATIONS

1DGE One dimensional gel electrophoresis (SDS-PAGE)
2DGE Two dimensional gel electrophoresis; IEF followed by SDS-PAGE
aa Amino acid
ab Antibody
ACN Acetonitrile; CH₃CN
AD Alzheimer's Disease
ADAM A disintegrin and metalloprotease
AFT AICD-Fe65-Tip60 complex
AFT AICD/Fe65/Tip60 (tripartite nuclear complexes)
AICD APP intracellular domain
Amp Ampicillin
APP Amyloid Precursor Protein
APS Ammonium persulfate
BD Binding domain
b-ME β-Mercaptoethanol
bp Base pairs
BPI Base peak ion
C13O18 Chromosome 13 ORF 18
CBP Calmodulin Binding Peptide
CFP Cyan Fluorescent Protein
CHCA Alpha-Cyano-4-Hydroxycinnamic Acid
CHO Chinese hamster ovary cells
CID collision induced dissociation
CIP Calf intestinal phosphatase
CLSM Confocal laser scanning microscopy
CMV Cytomegalovirus (strong eukaryotic promoter)
Ct Number of cycles required for a PCR product to be present at threshold level
DAPI 4,6-Diamidino-2-phenylindole; a stain for dsDNA used to visualize the nucleus
DAPT N—[N-(3,5-Difluorophenacetyl-L-alanyl)]-S-phenylglycine t-Butyl Ester; a γ-secretase inhibitor
DBD DNA binding domain
DEPC Diethylpyrocarbonate
DMEM Dulbecco's modified eagle medium
DMSO Dimethylsulfoxide
dsDNA Double stranded DNA
DST Disuccinimidyl-tartrate
DTT Dithiothreitol
ECL Electrochemiluminescence
ECM Extracellular matrix
EDTA Ethylenediamine-tetra-acetic acid
EGTA Ethyleneglycol-bis(diaminoethylether)-tetra-acetic acid
EL Eluate
EOAD Early onset AD, cf. FAD
ER Endoplasmic reticulum
EtOH Ethanol
FA formic acid
FAD Familial AD
FCS Fetal calf serum
flAPP Full-length APP
FT-ICR Fourier-Transform ion cyclotron resonance (MS), or FT(-MS) for short
Fulspec Full-scan based peak exclusion (algorithm)
GluFib Glu-Fibrionopeptide calibration peptide (EGVNDNEEGFFSAR)
HA Hemagglutinin (-tag)
HRP Horse-radish peroxidase
IEF Isoelectric focusing of proteins; separation based on pH-dependent charge
IP Immunoprecipitation
IPTG Isopropyl-β-D-galactopyranoside
Kan Kanamycin
KD knockdown
KLC Kinesin light chain
KO Knockout (incapacitated or deleted gene)
LB-medium Luria Bertani medium
LF Lipofectamine 2000 transfection reagent
LTP Long term potentiation
MALDI Matrix assisted laser desorption/ionization
MAPK Mitogen activated protein kinase (pathway)
MCI Mild cognitive impairment
MetOH Methanol
MMTS Methyl methanethiosulfonate
MS/MS Tandem mass spectrometry
MW Molecular weight
NFT Neurofibrillary tangle
NSF N-ethyl-maleimide sensitive factor
P/S Penicillin-Streptomycin antibiotic mixture
PAT Protein interacting with APP tail
PBS Phosphate buffered saline
PCR Polymerase chain reaction
PFA Paraformaldehyde
Pfu Pyrococcus furiosus
pI Isoelectric point
PMT Photomultiplier
ppm Parts per million
ProlR Prolactin Receptor
PS Presenilin, a component of γ-secretase
PTB Phospho-tyrosine binding (domain)
RA Retinoic acid
RIP Regulated intramembraneous proteolysis
RT Room temperature
SN Signal-to-noise ratio; reduced by n^{1/2} when averaged n-fold
SBP Streptavidin Binding Peptide
SDM Site directed mutagenesis
SDS-PAGE Sodium dodecyl sulfate polyacrylamide gel electrophoresis
SELDI Surface enhanced laser desorption ionisation
SN supernatant
SNAP Synaptosome-associated protein 25/soluble NSF attachment protein
SPA Sinapinic acid; a matrix reagent for SELDI-TOF of larger peptides or proteins
SREBP Sterol-response-element-binding protein
SS Silver staining
TA Transcription activating domain
TACE Tumor necrosis factor a converting enzyme
TAP Tandem affinity purification
TCEP Tris-(2-carboxyethyl) phosphine
TEMED Tetramethyl-ethylenediamine
TFA Trifluoroacetic acid
TIC Total ion count
Tm Melting temperature, a.k.a. annealing temperature
TOF Time of flight (TOF/TOF enables MS/MS)
TPP Transproteomic data analysis pipeline
TX100 Triton X-100
WB Western Blotting
wt Wildtype
Y2H Yeast-2-hybrid system, protein interaction screening system
YFP Yellow Fluorescent Protein; enhanced version: Citrine

### DEFINITIONS

Unless stated otherwise, a term as used herein is given the definition as provided in the Oxford Dictionary of Biochemistry and Molecular Biology, Oxford University Press, 1997, revised 2000 and reprinted 2003, ISBN 0 19 850673 2.

“Neurodegenerative, neurological or neuropsychiatric disorders” include but are not limited to Alzheimer's Disease, mild cognitive impairment, fronto-temporal dementia, Lewy-body disease, Parkinson's disease, Pick's disease, Binswanger's disease; congophilic amyloid angiopathy, Down's syndrome, multi-infarct dementia, Huntington's Disease, Creutzfeldt-Jakob Disease, AIDS dementia complex, depression, anxiety disorder, phobia, Bell's Palsy, epilepsy, encephalitis, multiple sclerosis; neuromuscular disorders, neurooncological disorders, rbain tumors, neurovascular disorders including stroke, neuroimmunological disorders, neurootological disease, neurotrauma including spinal cord injury, pain including neuropathic pain, pediatric neurological and neuropsychiatric disorders, sleep disorders, Tourette syndrome, other movement disorders and disease of the central nervous system (CNS) in general. Unless stated otherwise, the terms neurodegenerative, neurological or neuropsychiatric are used interchangeably herein.

“Variant”, as the term is used herein, generally refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of a polypeptide or protein. “Variants” include, for example, proteins with conservative amino acid substitutions in highly conservative regions.

“Level”, as the term is used herein, generally refers to a gage of, or a measure of the amount of, or a concentration of a transcription product, for instance an mRNA, or a translation product.

“Activity”, as the term is used herein, generally refers to a measure for the ability of a transcription product or a translation product to produce a biological effect or a measure for a level of biologically active molecules. The terms “level” and/or “activity” as used herein further refer to gene expression levels, gene activity, or enzyme activity.

“Derivative”, as the term is used herein, generally refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or, acetylation, or lipidation, or by altered signal peptide cleavage or other types of maturation cleavage. These processes may occur post-translationally.

“Modulator”, as the term is used herein, generally refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene.

“Oligonucleotide primer” or “primer”, as the terms are used herein, generally refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all-possible sequences in a mix. The length of primers used herein may vary from 10 nucleotides to 80 nucleotides.

“Probes”, as the term is used herein, generally refers to short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample.

“Agent”, “reagent”, or “compound”, as the terms are used herein, generally refer to any substance, chemical, composition, or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or inorganic molecules or compositions, small molecules, drugs and any combinations of any of said agents above. They may be used for testing, for diagnostic or for therapeutic purposes.

If not stated otherwise the terms “compound”, “substance” and “(chemical) composition” are used interchangeably herein and include but are not limited to therapeutic agents (or potential therapeutic agents), food additives and nutraceuticals. They can also be animal therapeutics or potential animal therapeutics. Compounds to be screened may also be obtained from diversity libraries, such as random or combinatorial peptide or non-peptide libraries. Many libraries are known in the art that can be used, e.g., chemically synthesized libraries, recombinant (e.g., phage display libraries), and in vitro translation-based libraries.

Examples of chemically synthesized libraries are described in Fodor et al., Science 251 (1991), 767-773; Houghten et al., Nature 354 (1991), 84-86; Lam et al., Nature 354 (1991), 82-84; Medynski, Bio/Technology 12 (1994), 709-710; Gallop et al., J. Medicinal Chemistry 37(9), (1994), 1233-1251; Ohlmeyer et al., Proc. Natl. Acad. Sci. USA 90 (1993), 10922-10926; Erb et al., Proc. Natl. Acad. Sci. USA 91 (1994), 11422-11426; Houghten et al., Biotechniques 13 (1992), 412; Jayawickreme et al., Proc. Natl. Acad. Sci. USA 91 (1994), 1614-1618; Salmon et al., Proc. Natl. Acad. Sci. USA 90 (1993), 11708-11712; international application WO93/20242; and Brenner and Lerner, Proc. Natl. Acad. Sci. USA 89 (1992), 5381-5383.

Examples of phage display libraries are described in Scott and Smith, Science 249 (1990), 386-390; Devlin et al., Science 249 (1990), 404-406; Christian et al., J. Mol. Biol. 227 (1992), 711-718; Lenstra, J. Immunol. Meth. 152 (1992), 149-157; Kay et al., Gene 128 (1993), 59-65; and international application WO94/18318. In vitro translation-based libraries include but are not limited to those described in international application WO91/05058; and Mattheakis et al., Proc. Natl. Acad. Sci. USA 91 (1994), 9022-9026.

By way of examples of non-peptide libraries, a benzodiazepine library (see e.g., Bunin et al., Proc. Natl. Acad. Sci. USA 91 (1994), 4708-4712) can be adapted for use. Peptide libraries (Simon et al., Proc. Natl. Acad. Sci. USA 89 (1992), 9367-9371) can also be used. Another example of a library that can be used, in which the amide functionalities in peptides have been permethylated to generate a chemically transformed combinatorial library, is described by Ostresh et al., Proc. Natl. Acad. Sci. USA 91 (1994), 11138-11142.

Screening the libraries can be accomplished by any of a variety of commonly known methods; see, e.g., the following references, which disclose screening of peptide libraries: Parmley and Smith, Adv. Exp. Med. Biol. 251 (1989), 215-218; Scott and Smith, Science 249 (1990), 386-390; Fowlkes et al., BioTechniques 13 (1992), 422-427; Oldenburg et al., Proc. Natl. Acad. Sci. USA 89 (1992), 5393-5397; Yu et al., Cell 76 (1994), 933-945; Staudt et al., Science 241 (1988), 577-580; Bock et al., Nature 355 (1992), 564-566; Tuerk et al., Proc. Natl. Acad. Sci. USA 89 (1992), 6988-6992; Ellington et al., Nature 355 (1992), 850-852; U.S. Pat. No. 5,096,815, U.S. Pat. No. 5,223,409, and U.S. Pat. No. 5,198,346; Rebar and Pabo, Science 263 (1993), 671-673; and international application WO94/18318.

“Small organic molecule”, as the term is used herein, refers to an organic compound [or organic compound complexed with an inorganic compound (e.g., metal)] that has a molecular weight of less than 3 kilodaltons, preferably less than 1.5 kilodaltons.

“Treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

“Subject”, as employed herein, generally relates to animals in need of therapy, e.g. amelioration, treatment and/or prevention of a neurodegenerative, neurological, neuropsychiatric, neoplastic or infectious disease. Most preferably, said subject is a human.

### DETAILED DESCRIPTION OF THE PRESENT INVENTION

In general, the present invention concerns methods for detecting interactions between central nervous system-associated proteins and their ligands. As disclosed in detail below, the methods of the present invention have been exemplified with respect to the amyloid precursor protein (APP). However, it will be understood that the teachings of the present invention are applicable to any other neurodegenerative disease, neurological or neuropsychiatric disease-associated proteins. These include, for example, also proteins which are known or have been identified in accordance with the method of the present invention to interact with APP. Thus, while the general applicability of the present invention will be acknowledged, its further illustration has been exemplified for APP for the sake of conciseness only. Accordingly, in one aspect, the present invention is directed to a method of identifying or obtaining a molecule interacting with a neurodegenerative, neurological or neuropsychiatric disorder-associated protein comprising:
(a) providing the neurodegenerative, neurological or neuropsychiatric disorder-associated protein or a fragment thereof containing a tag within a cell or tissue under conditions allowing complex formation;
(b) subjecting a sample of the cell or tissue to at least one purification step;
(c) isolating the complex purified in step (b), and optionally
(d) identifying the respective interacting molecule of the neurodegenerative, neurological or neuropsychiatric disorder-associated protein.

The present invention is based inter alia on the isolation and analysis of proteins that have been found to bind to APP in vivo making use of a novel transgenic mouse model and combining biochemical purification of tagged APP with mass spectrometry analysis; see infra, of the description and the experiments performed in accordance with the present invention.

During initial optimization experiments performed in accordance with the present invention and required for establishing efficient purification techniques, comparison of conventional immuno-precipitations with bait peptide-mediated pull-downs revealed the latter to be superior in performance. Surprisingly, the final establishment of a transgenic mouse model expressing APP containing a purification tag resulted in the possibility of isolating target proteins from brain as a physiologically relevant environment in a scalable manner. Accordingly, in a preferred embodiment, the neurodegenerative, neurological or neuropsychiatric disorder-associated protein is a member of the amyloid precursor protein (APP)/APP-like protein (APLP)-family, most preferably APP.

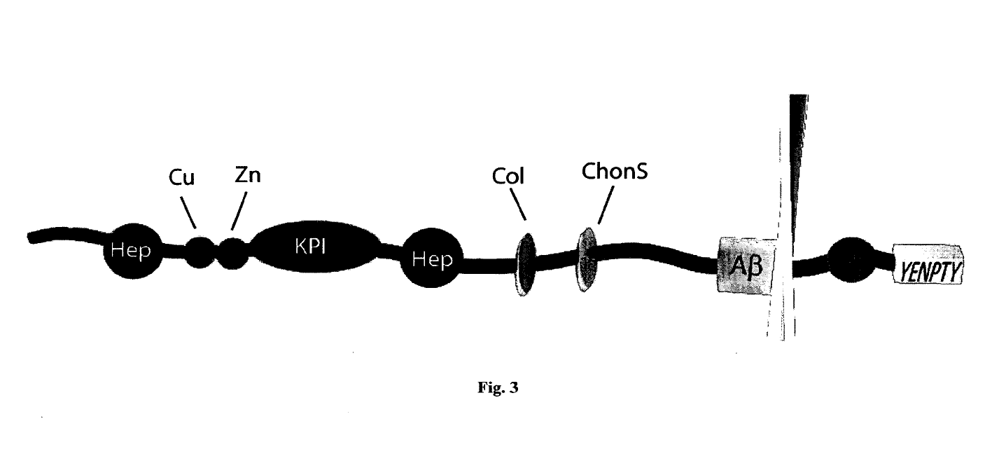

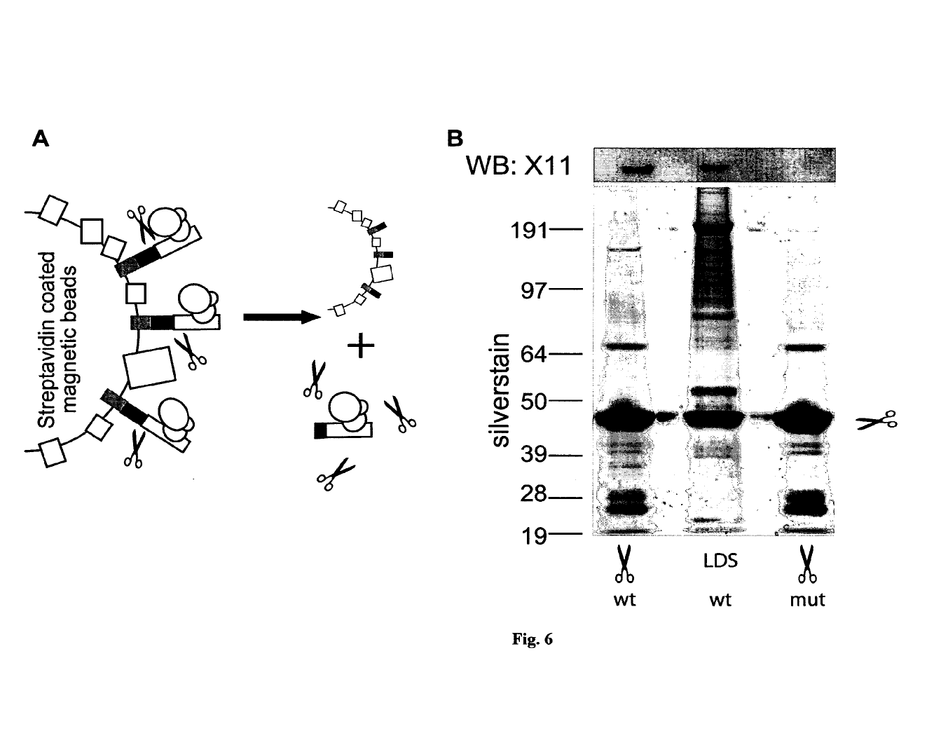

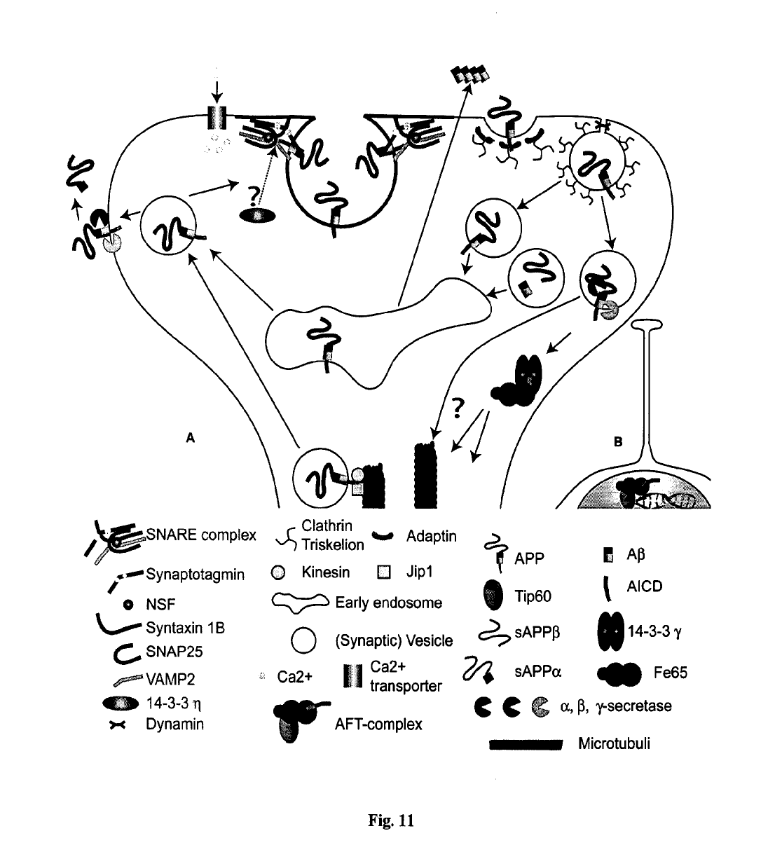

Quantitative proteomic analysis of samples from this mouse identified several proteins involved in synaptic vesicle endo- and exocytosis to be associated with APP: all components of the core SNARE-complex, which facilitates fusion of vesicles with the synaptic membrane, were identified, as well as several additional effectors as described in detail below; see also FIGS. 11, 6, as well as FIG. 3-5 of EP 06 024 239.2. These newly identified direct or indirect binding partners of APP represent new targets for therapeutic intervention. By way of example, the modification of the binding protein or of the direct or indirect complex formation of the binding protein with APP could lead to altered APP cellular localization, trafficking, signaling and reduced amyloidogenic processing, improved transport of neurotrophic factors, neuronal survival, synaptic placticity among others.

Dynamin, a key protein in cellular endocytosis, was identified by using the method described in this invention, and validated in additional cell culture experiments demonstrating that nuclear signaling of APP is impaired when endocytosis of APP is blocked by transfection of cells with a dominant negative Dynamin mutant that inhibits normal Dynamin function. Further, using a specific protease inhibitor and cleavage-inhibiting mutant forms of APP, the amyoloidogenic pathway of APP processing could be shown to play a significant role in translocation of the intracellular C-terminal domain of APP (AICD) to the nucleus and in transcriptional activation of AICD-regulated target genes including the genes encoding APP and its endoproteolytic secretase BACE. As a result of the new data generated by the present invention that identify Dynamin as a direct or indirect interaction partner of APP, novel strategies for treatment and prevention can be designed by supplying compounds that regulate expression of Dynamin or that modify the APP/Dynamin complex in order to reduce the formation of amyloidogenic APP derivatives, to decrease Amyloid formation, to reduce neurotoxicity, or to restore physiologic APP trafficking and signaling.

In addition to the APP binding proteins identified here and that are in involved in endo- and exocytosis and vesicular transport, a large number of further APP binding partners that fall into different functional categories were identified by using the method described in this invention. The large majority of these proteins has not been previously described as direct or indirect binding proteins of APP, and, thus, are specifically subject of the present invention.

Ubiquitin carboxyl-terminal hydrolase isozyme L1 (UCHL1_MOUSE, Primary accession number Q9R0P9; UCHL1_HUMAN, Primary accession number P09936) is a neuronal enzyme involved in recycling of ubiquitin, making it available for re-use in further cycles of tagging and targeted degradation of waste proteins in the proteasome. The brains of AD patients show an accumulation of ubiquinated proteins (de Vrij et al., Prog Neurobiol. (2004) 74:249-270), suggesting inhibition of the protein degradation machinery. UCH-L1 is associated with rare cases of Parkinson's disease (Lincoln et al., Neuroreport. 1999; 10(2):427-9.) and was reported to be down regulated in brains of sporadic AD cases (Choi et al., J Biol. Chem. 2004 Mar. 26; 279(13):13256-64.). In a recent study UCHL1 was shown to restore normal enzymatic activity and synaptic function both in hippocampal slices treated with oligomeric Abeta and in the APP/PS1 mouse model of AD and to improve the retention of contextual learning in APP/PS1 mice over time. The beneficial effect of the UCH-L1 fusion protein is associated with restoration of normal levels of the PKA-regulatory subunit IIalpha, PKA activity, and CREB phosphorylation (Gong et al., Cell 126 (2006), 775-788). The new finding that UCHL1 can form a complex with APP in vivo, either directly or indirectly, opens the way to novel strategies for treatment and prevention, kits, arrays etc. By way of example, these could include compounds that modify the APP/UCHL1 complex or the enzymatic activity of UCHL1 or modulate the binding of proteins to APP-bound UCHL1 or the activity of these proteins, and that lead to changes in APP processing, amyloid formation and reduce neurotoxicity, or restore physiologic APP turnover and degradation, trafficking and signaling.

Phosphatidylethanolamine binding protein (PEBP1_MOUSE, Primary accession number P70296; PEBP1_HUMAN, Primary accession number P30086) is a multifunctional protein, with proposed roles as the precursor protein of hippocampal cholinergic neurostimulating peptide (HCNP), and as the Raf kinase inhibitor protein (RKIP). PEBP mRNA has been reported to be decreased in the hippocampus in AD and Tg2576 transgenic AD model mice with a significant correlation between decreased PEBP expression and accumulation of Abeta (George et al., Neurobiol. Aging. 27 (2006), 614-623). PEBP was recently described as a novel calpain substrate and an inhibitor of the proteasome (Chen et al., J. Neurochem. 99 (2006), 1133-1141). In that study, PEBP levels were demonstrated to be greater in AD compared to healthy controls. Moreover, the membrane phospholipid phosphatidylethanol-amine is increased in brains obtained from patients with Alzheimer's disease, combined with increases in brain levels of its water-soluble metabolite glycerophosphoethanolamine (Nitsch et al., PNAS 1992) suggesting a role of membrane phospholipid turnover, and in particular, phosphatidylethanolamine in the disease and in APP processing. The new findings, generated by the present invention, that identify PEBP as a direct or indirect interaction partner of APP in vivo, opens the way to novel strategies for treatment and prevention. By way of example, these could be supplying PEBP or mimetics thereof or compounds that regulate expression of PEBP or modify the APP/PEBP complex designed to reduce the formation of amyloidogenic APP derivatives, to decrease amyloid formation, to reduce neurotoxicity, or to restore physiologic APP turnover, trafficking and signaling.

PEP-19 (PEP19_MOUSE, Primary accession number P63054, PEP19_HUMAN, Primary accession number P48539) is a 61-amino-acid neuronal calmodulin-binding protein encoded by the PCP4 gene (Ziai, Proc Natl Acad Sci USA. (1986) 83:8420-3) and located in the critical region of human chromosome 21 that is triplicated in Down syndrome, and thus causes brain amyloidosis and Alzheimer's disease pathology via increasing the APP gene dose. Cerebellar hypoplasia is a feature of Down syndrome, and it has been hypothesized that overexpression of PEP-19 contributes to this aspect of the disorder (Cabin et al., Somat Cell Mol Genet (1996) 22:167-75 1996). PEP-19 levels in the basal ganglia are markedly reduced in Huntington's disease (Utal et al., Neuroscience (1998) 86, 1055-63). In contrast, cerebellar PEP-19 levels are increased in Alzheimer disease (Slemmon et al., J. Neurosci. (1994) 14:2225-35). Overexpression of PEP-19 in PC12 cells reduces their apoptotic responses to noxious stimuli, suggesting that PEP-19 has anti-apoptotic properties in neurons. As a result of the new data generated by the present invention that identify PEP-19 as a direct or indirect interaction partner of APP, novel strategies for treatment and prevention can be designed by supplying compounds that regulate expression of PEP-19 or that modify the APP/PEP-19 complex in order to reduce the formation of amyloidogenic APP derivatives, to decrease amyloid formation, to reduce neurotoxicity, or to restore physiologic APP turnover, trafficking and signaling.

Profilin (PROF1_MOUSE, Primary accession number P62962; PROF1_HUMAN, Primary accession number P07737) regulates actin polymerization by binding to the actin monomer (G-actin) and enhancing the ADP-ATP exchange on G-actin, thereby increasing the pool of ATP-actin in the cell (Witke, Trends Cell Biol. 14 (2004), 461-469). Profilin can therefore promote the elongation of the growing actin filament. Profilin is translocated into dendritic spines in cultured hippocampal neurons after neuronal stimulation, long-term potentiation (LTP) and long-term depression (Ackermann & Matus, Nat. Neurosci. 6 (2003), 1194-1200; Neuhoff et al., Eur. J. Neurosci. 21 (2005), 15-25). The translocation of profilin is associated with the suppression of actin dynamics in the spine head and the stabilization of spine morphology. A role of profilin in learning and memory was recently suggested by Lamprecht et al. (Nat. Neurosci. 9 (2006), 481-483) who showed that conditioning in rats leads to the movement of profilin into dendritic spines in the amygdala. These spines undergo enlargements in their postsynaptic densities which was hypothesized to contribute to the enhancement of synaptic responses in the lateral amygdala following fear learning. A similar function in the regulation of synaptic plasticity and fear learning has been suggested for myosin light chain kinase (MYLK2_MOUSE, Primary accession number Q8VCR8; MYLK2_HUMAN, Primary accession number Q9H1R3) which was also identified in the present screen (Lamprecht et al., 2006 Neuroscience 139 (2006), 821-829). The new findings generated by the present invention that identify both profilin and myosin light chain kinase as direct or indirect interaction partners of APP in vivo, open the way to novel strategies of treatment. By way of example, these could be supplying profilin or myosin light chain kinase or mimetica thereof or compounds that modify the APP/profiling or APP/myosin light chain kinase complex in order to restore synaptic function or reduce the formation of amyloidogenic APP derivatives, to decrease amyloid formation, to reduce neurotoxicity, or to restore physiologic APP turnover, trafficking and signaling.

AD-related abnormalities in glutamatergic signaling have been attributed to excitotoxicity caused by the persistent, low-level stimulation of glutamatergic neurons via the chronic influx of Ca(2+) ions through the N-methyl-D-aspartate receptor calcium channel. The present screen identified two glutamate transporters, Excitatory amino acid transporter 1 (EAA1_MOUSE, Primary accession number P56564; EAA1_HUMAN, Primary accession number P43003) and Excitatory amino acid transporter 2 (EAA2_MOUSE, Primary accession number P43006; EAA2_HUMAN, Primary accession number P43004) which are required for the termination of signal transmission mediated by glutamate as well as for the prevention of neurotoxicity mediated by this endogenous excitotoxin. The here identified direct or indirect APP/glutamate transporter complexes are potential targets for therapeutic interference for different neurodegenerative, neurologic or neuropsychiatric disorders related to malfunction of glutamate signaling, as well as for reducing excitotoxic neuronal damage in such conditions. Moreover, such therapeutic interventions can be designed to reduce the formation of amyloidogenic APP derivatives, to decrease amyloid formation, to restore physiologic APP turnover, trafficking and signaling.

The novel APP interacting proteins peroxiredoxin 5 (PRDX5_MOUSE, Primary accession number P99029; PRDX5_HUMAN, Uniprot accession number P30044) and Superoxide dismutase [Cu—Zn] (SODC_MOUSE, Primary accession number P08228; SODC_HUMAN, Primary accession number P00441) play an important role in the detoxification of free radicals and prevention of oxidative stress which is believed to be a key factor in the pathogenesis of neurodegenerative diseases. Both proteins are candidate targets for the compounds that reduce oxidative neuronal damage as well as to reduce the formation of amyloidogenic APP derivatives, to decrease amyloid formation, or to restore physiologic APP turnover, trafficking and signaling.

The leucine-rich repeat kinase 1 (LRRK1_MOUSE, Primary accession number Q3UHC2; LRRK1_HUMAN, Primary accession number Q38SD2) is a multi-domain protein of unknown function belonging to the ROCO family of complex proteins containing a functional protein kinase and a GDP/GTP-binding protein. LRRK1 is closely related to human LRRK2/dardarin, a ROCO protein and putative serine/threonine kinase which has been linked to the pathogenesis of Parkinson's disease (Bosgraaf and Van Haastert, J. Biochim. Biophys. Acta 1643 (2003), 5) the only human paralogue of LRRK1, that has been linked to autosomal-dominant parkinsonism. The present finding suggests that LRRK1 may play a role in neurodegenerative diseases and, therefore, is a candidate drug target for compounds designed to influence phosphorylation of disease-related proteins. In addition, such compounds may be designed to reduce the formation of amyloidogenic APP derivatives, to decrease amyloid formation, to reduce neurotoxicity, or to restore physiologic APP turnover, trafficking and signaling

Cyclophilin A (PPIA_MOUSE, Primary accession number P17742; PPIA_HUMAN, Primary accession number P62937) is a member of a large group of small molecular weight proteins that are highly conserved from micro-organisms to humans. A key feature of cyclophilin A is its cistrans peptidyl prolyl isomerase, which catalyzes the isomerization of the peptide bond between pSer/Thr-Pro in proteins, thereby regulating their biological functions which include protein assembly, folding, intracellular transport, intracellular signaling, transcription, cell cycle progression and apoptosis. Another peptidyl-prolyl isomerase, Pint (PIN1_MOUSE, Primary accession number Q9QUR7; PIN1_HUMAN, Primary accession number Q13526), has been shown to co-localize with phosphorylated tau in AD brain, and shows an inverse relationship to the expression of tau. Pin1 protects neurons under in vitro conditions. Recent studies demonstrate that APP is a target for Pin1 and Pin1 can regulate both APP processing and Aβ production (Pastorino, Nature (2006) 440:528-34). Furthermore, Pin1 was found to be oxidatively modified and to have reduced activity in the hippocampus in mild cognitive impairment and AD. Because of the diverse functions of Pin1, and the discovery that this protein is one of the oxidized proteins common to both MCI and AD brain, the question arises as to whether Pin1 is one of the driving forces for the initiation or progression of AD pathogenesis, finally leading to neurodegeneration and neuronal apoptosis. The present findings suggest that the cis-trans peptidyl prolyl isomerase cyclophilin A which can form a direct or indirect complex with APP may have a function similar to Pin1 and therefore is a candidate drug target for the treatment of AD and neurodegenerative disorders. Compounds that interact with cyclophylin A or modify the APP/cyclophylin A complex may be designed to reduce the formation of amyloidogenic APP derivatives, to decrease amyloid formation or tau phosphorylation and aggregation, to reduce neurotoxicity, or to restore physiologic APP turnover, trafficking and signaling.

The present screen identified a number of mitochondrial energy metabolism associated proteins as novel binding partners of APP. These include several subunits of the mitochondrial ATP synthase. Of this multisubunit membrane-bound complex that couples the transmembrane proton motive force to the synthesis of ATP from ADP and orthophosphate (Boyer, Biochim. Biophys. Acta, 1365 (1998), 3-9) the alpha, beta, gamma and epsilon chains were identified in the present screen (ATPA_MOUSE, Primary accession number Q03265; ATPA_HUMAN, Primary accession number P25705; ATPB_MOUSE, Primary accession number P56480; ATPB_HUMAN, Primary accession number P06576; ATPG_MOUSE, Primary accession number Q91VR2; ATPG_HUMAN, Primary accession number P36542; ATP5I_MOUSE, Primary accession number Q06185; ATP5I_HUMAN, Primary accession number P56385).

In addition, two enzymes of the Krebs cycle were found to be in a complex with APP: aconitate hydratase (ACON_MOUSE, Primary accession number Q99KI0; ACON_HUMAN, Uniprot accession number Q99798) and malate dehydrogenase (MDHC_MOUSE, Primary accession number P14152; MDHC_HUMAN, Uniprot accession number P40925). In addition to its enzymatic activity, aconitate hydratase can function as an iron sensitive RNA-binding protein that regulates the translatability or stability of certain transcripts.

These findings suggest that APP may have a function in energy metabolism and mitochondrial function which could be modulated by pharmacological intervention. Previous work has shown that APP carries a dual leader sequence and can be targeted to mitochondria and that transmembrane-arrested APP is associated with reduced cytochrome oxidase activity, decreased ATP synthesis and loss of the mitochondrial membrane potential (Devi et al., J. Neurosci. 26 (2006), 9057-9068). In post-mortem brain samples, nonglycosylated full-length and C-terminally-truncated APP was shown to be associated with mitochondria of Alzheimer disease samples, but not healthy controls. The levels of mitochondrial APP increase with disease severity and may contribute to the disease progression e.g. by modifying mitochondrial function. Pharmacological interventions could by way of example target the complex formation with the newly identified mitochondrial APP binding proteins and thereby prevent translocation of APP to the mitochondria, restore normal ATP synthase function, restore normal mitochondrial trafficking and subcellular localization, prevent mitochondrial decay by oxidative modification of key mitochondrial enzymes, restore normal function of key mitochondrial enzymes and reduce amyloid formation in mitochondria.

In addition, a large number of enzymes involved in glycolysis were identified, further suggesting that APP may have a function in energy metabolism that could be modulated by pharmacological intervention. These glycolytic enzymes include neuron-specific enolase (ENOG_MOUSE, Primary accession number P17183; ENOG_HUMAN, Primary accession number P09104), alpha-enolase (ENOA_MOUSE, Primary accession number P17182; ENOA_HUMAN, Primary accession number P06733), aldolase 1 (ALDOA_MOUSE, Primary accession number P05064; ALDOA_HUMAN, Primary accession number P04075); aldolase 3 (ALDOC_MOUSE, Primary accession number 05063; ALDOC_HUMAN, Primary accession number P09972), phosphoglycerate mutase (PGAM1_MOUSE, Primary accession number Q9DBJ1; PGAM1_HUMAN, Primary accession number P18669), pyruvate kinase isozyme M1/2 (KPYM_MOUSE, Primary accession number P52480; KPYM_HUMAN, Primary accession number P14618), triosephosphate isomerase (TPIS_MOUSE, Primary accession number P17751; TPIS_HUMAN, Primary accession number P60174), glucose-6-phosphate isomerase (G6P1_MOUSE, Primary accession number P06745; G6PI_HUMAN, Primary accession number P06744), lactate dehydrogenase B chain (LDHB_MOUSE, Primary accession number P16125; LDHB_HUMAN, Primary accession number P07195) and glyceraldehyde-3-phosphate dehydrogenase (G3P_MOUSE, Primary accession number P16858; G3P_HUMAN, Primary accession number P04406). Of these, only glyceraldehyde-3-phosphate dehydrogenase has previously been reported to be found in a complex with APP (Schulze, J. Neurochem. 60 (1993) 1915-1922). Brain imaging studies have demonstrated deficits in glucose utilization in AD patients and the activities of critical enzymes in energy metabolism are decreased in brain cells of AD patients (Blass, J. Neurosci. Res. 66 (2001) 851-856). Our finding suggest that APP may contribute to impaired energy metabolism in AD and opens new vistas for therapeutic intervention.

Elongation factor 1-alpha 2 (EF1A2_MOUSE, Primary accession number P62631; EF1A2_HUMAN, Primary accession number Q05639) plays an important role in translation by catalyzing GTP-dependent binding of aminoacyl-tRNA to the acceptor site of the ribosome. However, several studies have implied other functions of the protein as well. EF1A has been shown to bind and bundle actin and to sever microtubules. Furthermore, it has been reported to act as an activator of phosphoinositol 4-kinase and play a part in ubiquitin-dependent degradation of Na-acetylated proteins. As a result of the new data generated by the present invention that identify EF1A2 as a direct or indirect interaction partner of APP, novel strategies for treatment and prevention can be designed by supplying compounds that regulate expression of EF1A2 or that modify the APP/EF1A2 complex in order to reduce the formation of amyloidogenic APP derivatives, to decrease amyloid formation, to reduce neurotoxicity, or to restore physiologic APP turnover, trafficking and signaling.

Proteolipid protein PLP/dm-20 (GPM6B_MOUSE, Primary accession number P35803, GPM6B_HUMAN, Primary accession number Q13491]) belongs to the dm family of genes (Yan et al., Neuron 11 (1993) 423-31). Plp encodes two alternative spliced products: the proteolipid protein (PLP) and DM-20, which are proteins with four putative transmembrane domains and are the major protein components of higher CNS myelin. Missense mutations in the human PLP1 gene lead to dysmyelinating diseases with a broad range of clinical severity, ranging from severe Pelizaeus-Merzbacher disease to milder spastic paraplegia type 2. The molecular pathology has been generally attributed to endoplasmic reticulum retention of PLP and its splice isoform DM20 and induction of the unfolded protein response. As a result of the new data generated by the present invention that identify Proteolipid protein as a direct or indirect interaction partner of APP, novel strategies for treatment and prevention can be designed by supplying compounds that regulate expression of Proteolipid protein or that modify the APP/Proteolipid protein complex in order to reduce the formation of amyloidogenic APP derivatives, to decrease amyloid formation, to reduce neurotoxicity, or to restore physiologic APP turnover, trafficking and signaling.

The findings of the present invention show that regulated intramembraneous proteolysis of APP may have two different outcomes, depending on the substrate. Therefore, indiscriminate inhibition of beta-secretase, a drug target that is responsible for the first cleavage step during the production of Abeta, may lead to side-effects by also blocking AICD-signaling. In addition, several novel protein interaction partners of APP were found which opens new vistas for further studying APP function and therapeutic intervention in the treatment of AD.

As already mentioned supra, the method of the present invention makes use of a tagged APP, said tag preferably being streptavidin-binding peptide (SBP) as described in detail infra; see also legend to table 5. However, other tags known in the art can of course be used as well such as for example an N-terminal FLAG-tag, glutathione-5-transferase (GST), or a 6*His-tag, myc-tag, Fc-tag, CBP-tag and the like.

Furthermore, as described in detail below, complexes comprising APP and the interacting molecule and the interacting molecule alone, respectively, can conveniently be analyzed using mass spectroscopy. Accordingly, step (d) of the method of the present invention is intended to usually comprise mass spectroscopy, preferably matrix assisted laser desorption/ionization-time of flight/time of flight (MALDI-TOF/TOF) or mass spectroscopy comprising ion trap and Fourier Transformation (LTQ-FT) as used in the experiments described below. According to the experiments performed in accordance with the present invention, prior to the MALDI-TOF/TOF analysis a further labeling step such as iTRAQ labeling can be performed as described in detail for example in Example 3. However, the person skilled in the art knows that other labeling methods can be used as well as long as they provide comparable success.

Furthermore, neurodegenerative, neurologic and neuropsychiatric diseases, in particular Alzheimer's disease, mainly affect the brain and cells and tissues being in direct contact therewith, respectively. Thus, the samples used in the method of the present invention preferably comprise cells and tissues, respectively, of or in more or less direct contact with the brain. In a preferred embodiment the samples used in accordance with the method of the present invention comprise brain homogenate, brain sections, cerebral spinal fluid or cells of the brain or CNS.

In principle, the cells and tissues to be used in the method of the present invention can be derived from humans or animals, for example in the form of a cell or tissue culture, wherein in the respective cells is provided, either by endogenous expression or exogenous addition to, a central nervous system-associated protein, i.e. here APP. Cell and tissue culture techniques as well as stable and transient expression of recombinant proteins in cell and tissue cultures are well known to the person skilled in the art and may be found in the literature referred to in context with the detailed description of the experiments. Furthermore, the person skilled in the art is well aware of methods of providing proteins and other constituents exogenously to a cell or tissue so as to have them enter the cell and exert the desired effect. However, as demonstrated in the experiments performed in accordance with the present invention and further described infra and in particular in Examples 2 and 7, the cell or tissue is preferably comprised in or derived from a transgenic animal, preferably a transgenic mouse.

As described above, the present invention focuses on the interaction of the amyloid precursor protein (APP) with its interacting molecules such as APP's natural ligand proteins. A general overview of APP, its physiological function, its functional regions, its structure/set up and further characteristics is given in detail infra.

There are several conceivable ways to provide APP in the context and for the purpose of binding experiments, i.e. for the identification of potential binding and interacting partners, respectively. However, according to the experiments performed in accordance with the present invention and further described in detail below, the APP is preferably provided within the cell or tissue of a non-human animal, thereby not only preventing artificial effects due to for example contaminations, but also providing a physiological relevant environment.

According to a most preferred embodiment of the method of the present invention and described in detail below and in Example 7, the APP or a fragment thereof is provided within the cell or tissue by its recombinant expression in the non-human animal, preferably in a transgenic animal such as a transgenic mouse.

Although several transgenic mouse models of various aspects of Alzheimer's disease pathology are known, the present invention provides and makes use of a novel transgenic mouse, which is suitable and especially designed for use in the methods of the present invention. A general overview will be provided infra. Thus, in accordance with the experiments performed within the scope of the present invention the transgenic mouse is preferably the APP-TAP-AICD mouse.

As will be discussed below, most methods used in the field of proteomics comprise at least one purification step such as for example gel-elution, chromatography steps, precipitation, in particular immunoprecipitation, washing or centrifugal proceedings such as fractionated centrifugation, the variety of which is known to the person skilled in the art. However, according to the experiments performed in accordance with the present invention, purification step (b) of the method preferably essentially consists of an affinity purification, most preferably of purification via streptavidin; see infra. In a particularly preferred embodiment, the method of the present invention comprises step (c) or (d) immediately following step (b) without any further substantial purification step. This particular embodiment of the method of the present invention is most suitable for isolating the target protein or other molecules binding to the neurodegenerative, neurological or neuropsychiatric disorder-associated protein, here APP. This holds especially true for the method being performed as described in the detailed description and the experimental section below. More specifically, in accordance with the present invention, it was surprisingly found that this substantially one purification step method is superior to conventional methods which make use of at least two purification steps such as those initially tested for the purposes of the present invention; see the detailed description below. Thus, besides the advantage of having only one purification step resulting in high recovery of the putative complex of the disease associated protein and its binding target molecule or the target molecule alone, this embodiment of the method of the present invention is most reliable and easy to perform.

As already mentioned, the method of the present invention is designed to identify and obtain APP-interacting molecules. Therefore, from the conception of the method it is clear that by its successful use, i.e. identification of interacting molecules such as natural interacting molecules of APP, the molecules so identified may comprise molecules which are already known to bind to APP, the identification and isolation of which counts for the quality and reliability of the method of the present invention to truly identify APP interacting molecules. This is a further validation and not at least quality characteristic as well as proof of concept for the successful working of the method of the present invention; see infra. In this context, the proteins or other molecules hitherto known to bind to APP are not encompassed within the scope of the present invention. This particularly applies to any protein and other molecule which is described or mentioned in the documents cited herein.

In a further aspect the present invention relates to a complex and interacting molecule, respectively, obtainable by the method of the present invention, preferably wherein the interacting molecule is a molecule hitherto not disclosed in the prior art to interact with APP or a fragment thereof or not yet purified. In a preferred embodiment, said molecule is a protein or peptide, more preferably said protein is selected from the group consisting of proteins given in tables 1, 2, 4, 5, 13 and 14 in the description, and more preferably (P56564) excitatory amino acid transporter (GLAST), (P62962) profilin-1, (P70296) phosphatidylethanolamine-binding protein (PEBP), elongation factor 1-alpha 2 (EF-1-alpha-2), (P99029) peroxiredoxin 5, (P08228) superoxide dismutase [Cu—Zn], (Q8VCR8) myosin light chain kinase 2, skeletal/cardiac muscle (MLCK2), (P63054) brain-specific polypeptide PEP-19, serine/threonine-protein phosphatase 2A 65 kD regulatory subunit A, (Q3UHC2) leucine-rich repeat kinase 1 (LRRK1), synaptosomal-associated protein 25 (SNAP-25), neuronal membrane glycoprotein M6-b (M6b), N-ethylmaleimide sensitive fusion protein (NSF), plasma membrane calcium-transporting ATPase 2 (PMCA2), Ras-related protein Rab-1A (YPT1-related protein), clathrin coat assembly protein Aβ180, dynamin-1, (Q9R0P9) ubiquitin carboxyl-terminal hydrolase isozyme L1 (UCH-L1), (P61264) syntaxin-1B2, (P43006) excitatory amino acid transporter 2 (GLT-1), (P63044) vesicle-associated membrane protein 2 (VAMP-2), (P46096) synaptotagmin-1, (Q62419) SH3-containing GRB2-like protein 1, (P17742) peptidyl-prolyl cis-trans isomerase A (rotamase), (P05213) tubulin alpha-2 chain (alpha-tubulin 2), or (Q9D6F9) tubulin beta-4 chain, (P35803) proteolipid protein PLP/dm-20, (P62631) elongation factor 1-alpha 2 (EF1A2) and the mitochondrial ATP synthase subunits e.g. the alpha, beta, gamma and epsilon chains (Q03265, P56480, Q91VR2, Q06185, P56385).

Once APP interacting molecules such as its natural ligands are identified by the method of the present invention, their interaction with APP may be modulated, i.e. blocked, enhanced, facilitated, hampered or the like by for example exposing the cell, tissue, APP itself or the interacting molecule to compounds, i.e. test compounds which are capable of mediating those effects. The way how they act, i.e. their mode of action can be different. For example a test compound may either bind to
(a) the APP itself
   (i) at that site of the APP, which is normally responsible for the interaction with the corresponding partner;
   (ii) at a site, usually not directly involved in the interaction with a potential interacting molecule, but by binding to that site changing APP's conformation leading either to disappearance or alteration of the binding site and as a consequence preventing the afore-mentioned interaction; or it binds to
(b) the interacting partner, preferably identified by the method of the present invention, wherein binding occurs according either to (i) or (ii).

Thus, in case of for example inhibition of the interaction, the test compounds may act for example either as competitive or allosteric inhibitors. Furthermore, the modulating compound may act not by preventing an interaction but by disturbing an already formed binding.

Therefore, in a further aspect, the present invention relates to a method of identifying or obtaining compounds capable of modulating the binding of APP or a fragment thereof to its natural interacting molecule, comprising the steps of the method used for identification and obtaining the interacting partner, as described herein. Preferably, the test compound or a collection of test compounds is subjected to the cell or tissue or a sample thereof prior, during or after complex formation between APP or a fragment thereof with its putative interacting molecule. In another preferred embodiment the test compound is selected for its capability of modulating the binding of APP or a fragment thereof to its natural interacting molecule and/or modifying the enzymatic activity of the interacting molecule. Preferably, the natural interacting molecule is a protein as defined supra, in particular any one of those identified in accordance with the method of the present invention and described below.

Several strategies have been described in the prior art to detect and monitor, respectively, binding between molecules, and as a consequence detecting inhibition or modulation of said binding, respectively, which may be used in accordance with the present invention. Those strategies comprise for example tagging at least one partner with molecules the properties of which change upon binding such as illuminating molecules, wherein the detected signal might be light emittance such as fluorescence increase or decrease, or gaining additional or loosing former properties upon binding. Those strategies may of course also be used in accordance with the present invention, i.e. to detect and control, respectively, binding or non-binding of APP to its interacting molecule. Concerning the screening applications of the present invention relating to the testing of pharmaceutical compounds in drug research, it is generally referred to the standard textbook “In vitro Methods in Pharmaceutical Research”, Academic Press, 1997. In general, according to the present invention, the decrease of complex formation compared to performing the method without the test compound or collection of test compounds is indicative for a putative drug.

Hence, the present invention provides a number of viable targets for screening drugs that are expected to interfere with APP related pathogenesis and thus hold great promise as potential therapeutics which ameliorate APP-related disorders. Potential modulators include small organic molecules that mimic the function of first messengers, and/or analogs thereof, inhibitors, and/or toxins that modulate the processes that effect the processing of APP, in particular the amyloidogenic pathway. Once a potential modulator is identified, chemical analogues can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively synthesized de novo. The prospective agent (drug) can be placed into any standard assay to test its effect on the processing and cellular trafficking of APP. A drug is then selected that preferably rescues and/or confers resistance to disorders mediated by the amyloidogenic pathway of APP and by Aβ-oligomer aggregation in particular.

Thus, the present invention also contemplates screens for small molecules, analogs thereof, as well as screens for natural modulators of APP processing such as those that bind to and inhibit APP or its interaction partner in vivo.

In one exemplary assay the target, e.g., profilin can be attached to a solid support. Methods for placing profilin on the solid support are well known in the art and include such things as linking biotin to profilin and linking avidin to the solid support. The solid support can be washed to remove unreacted species. A solution of a labeled potential modulator (e.g., an inhibitor) can be contacted with the solid support. The solid support is washed again to remove the potential modulator not bound to the support. The amount of labeled potential modulator remaining with the solid support and thereby bound to profilin can be determined. Alternatively, or in addition, the dissociation constant between the labeled potential modulator and profilin, for example can be determined. Suitable labels for either profilin or the potential modulator are well known in the art and include enzymes, fluorophores (e.g., fluorescene isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. In a particular embodiment, isothermal calorimetry can be used to determine the stability of the profilin-APP complex in the absence and presence of the potential modulator.

In another embodiment, a Biacore machine can be used to determine the binding constant of the profilin-APP complex in the presence and absence of the potential modulator. Alternatively, profilin can be immobilized on a sensor chip. APP can then be contacted with (e.g., flowed over) the sensor chip to form the profilin-APP complex. In this case the dissociation constant for the profilin-APP complex can be determined by monitoring changes in the refractive index with respect to time as buffer is passed over the chip. (O′Shannessy et al., Anal. Biochem. 212 (1993), 457-468; Schuster et al., Nature 365 (1993), 343-347). Scatchard Plots, for example, can be used in the analysis of the response functions using different concentrations of APP. Flowing a potential modulator at various concentrations over the profilin-APP complex and monitoring the response function (e.g., the change in the refractive index with respect to time) allows the dissociation constant for the profilin-APP complex to be determined in the presence of the potential modulator and thereby indicates whether the potential modulator is either a stabilizer, or destabilizer of the profilin-APP complex. In addition, or alternatively, a potential modulator of profilin can be examined through the use of computer modeling using a docking program such as GRAM, DOCK, or AUTODOCK (Dunbrack et al., Folding & Design 2 (1997), 27-42), to identify potential modulators of profilin. These modulators can then be tested for their effect on APP processing and trafficking. This procedure can include computer fitting of potential modulators to the profilin-APP complex to ascertain how well the shape and the chemical structure of the potential modulator will bind to either profilin, APP or to the profilin-APP complex (Bugg et al., Scientific American, Dec.: (1993), 92-98; West et al., TIPS, 16 (1995), 67-749). Computer programs can also be employed to estimate the attraction, repulsion, and steric hindrance of the subunits with a modulator/inhibitor (e.g., profilin-APP complex and a potential destabilizer). Generally, the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side-effects due to unwanted interactions with other proteins.

As mentioned, the present invention also relates to the use of therapeutic agents which bind to APP and are derived from an APP interacting protein identified in accordance with the present invention. Such agents include but are not limited to synthetic peptides derived from said proteins. Synthetic peptides can be prepared using the well known techniques of solid phase, liquid phase, or peptide condensation techniques, or any combination thereof, and can include natural and unnatural amino acids. Amino acids used for peptide synthesis may be standard Boc (N^{α}-amino protected N^{α}-butyloxycarbonyl)amino acid resin with the standard deprotecting, neutralization, coupling and wash protocols of the original solid phase procedure of Merrifield (J. Am. Chem. Soc., 85 (1963), 2149-2154) or the base-labile N^{α}-amino protected 9-fluorenylmethoxycarbonyl (Fmoc) amino acids first described by Carpino and Han (J. Org. Chem., 37 (1972), 3403-3409). Peptides of the invention may comprise D-amino acids, a combination of D- and L-amino acids, and various “designer” amino acids (e.g., β-methyl amino acids, Ca-methyl amino acids, and Na-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, α-helices, β-turns, β-sheets, γ-turns, and cyclic peptides can be generated.

Furthermore, the term “derived from an APP interacting protein” includes agents which bind to said APP interacting protein identified in accordance with present invention, for example interacting proteins or peptides, preferably other than APP, and antibodies or antibody-derived molecules in particular. Suitable antibodies are preferably monoclonal antibodies, but also synthetic antibodies as well as fragments of antibodies, such as Fab, Fv or scFv fragments etc. Antibodies or fragments thereof can be obtained by using methods which are described, e.g., in Harlow and Lane “Antibodies, A Laboratory Manual”, CSH Press, Cold Spring Harbor, 1988 or European patent application EP-A 0 451 216 and references cited therein. Surface plasmon resonance as employed in the BIAcore system can be used to increase the efficiency of phage antibodies which bind to an epitope of the APP interacting protein (Schier, Human Antibodies Hybridomas 7 (1996), 97-105; Malmborg, J. Immunol. Methods 183 (1995), 7-13). The production of chimeric antibodies is described, for example, in international application WO89/09622. Methods for the production of humanized antibodies are described in, e.g., EP-A1 0 239 400 and WO90/07861. Further sources of antibodies to be utilized in accordance with the present invention are so-called xenogeneic antibodies. The general principle for the production of xenogeneic antibodies such as human antibodies in mice is described in, e.g., international applications WO91/10741, WO94/02602, WO96/34096 and WO96/33735.

Thus, the compounds which can be identified by the above mentioned method to be capable of affecting the interaction of APP to its interacting molecules is not limited. However, in a preferred embodiment, the compound is a peptide, polypeptide, PNA, peptide mimetic, antibody, nucleic acid molecule, aptamer or small organic compound, capable of interfering with the interaction of APP or its fragment with a natural interacting molecule or substantially suppressing the endogenous expression of the gene encoding the interacting molecule. In addition, such compounds may be designed to reduce the formation of amyloidogenic APP derivatives, to decrease amyloid formation, to reduce neurotoxicity, or to restore physiologic APP turnover and functions including trafficking and signaling.

As mentioned above and described in detail below, the amyloidogenic pathway of APP processing plays a significant role in translocation of the intracellular C-terminal domain of APP (AICD) to the nucleus and in transcriptional activation of AICD target genes. Thus, the putative drug identified and obtained in accordance with the method of the present invention may have different biological activities, including but not limited to suppressing the production of Aβ, for example by influencing the conformation of APP necessary for α-, β- or γ-secretase cleavage, and/or blocking AICD-signaling, for example by interfering with the translocation of AICD to the nucleus. Most preferably, the drugs identified in accordance with the present invention exhibit one or more of the following properties, i.e. shifting β-cleavage of APP to α-cleavage and modulating binding of APP to a shuttle protein required for transport to enzymes, in particular secretases involved in APP processing. In one embodiment, the peptide, polypeptide or peptide mimetic is derived from a protein binding domain or antibody recognizing the natural interacting molecule.

In addition, besides the use of newly identified compounds the present invention also contemplates the validation and thus the use of agents which are known to bind to any one of said APP interacting proteins but hitherto have not been considered to be useful in the treatment of neurodegenerative, neurological or neuropsychiatric disorders, in particular Alzheimer's disease and amyloidogenic disorders. Such compounds may be easily retrieved from the literature concerning any one of the APP interacting proteins, for example in patent databases such as espacenet hosted by the European Patent Office or in databases of public literature, e.g. medline. In addition, the person skilled in the art may identify agents to be used in accordance with the present invention by screening so-called “primary databases” such as Genbank, EMBL or UniprotKB/Swiss-Prot for nucleotide and protein sequences, respectively, for example by entering the Accession Number or the IUPAC-nomenclature or the name of the protein as referenced in the tables below. By those means also the human counterparts of the mouse proteins can be easily identified. The nucleotide and amino acid sequences in the mentioned databases are usually annotated with corresponding citations which in term provide further information with respect to regulation of the corresponding genes and thus guidance for modulating agents to be used in accordance with the present invention. In addition, so called “secondary databases” can be used, for example “PROSITE”, “PRINTS”, “Pfam”, “INTER Pro”, “SCOP” or “CATH”, being database of protein families and domains, providing fingerprints as classification of sequences, or protein structures. A most suitable web interface allowed to start searching is provided by “Entrez” of NCBI and sequence retrieval system “SRS”, respectively. Often a search with keywords in “Google” will already be successful in identifying suitable sources of information.

For validating a putative drug, in conjunction with the above assays an animal model can be used to ascertain the effect of a potential agent on an Aβ or amyloidosis related condition. For example, locomotor behavioral response or long term potentiation (LTP) of the animal can be determined in the presence and absence of the agent. For appropriate animal models see, for example (Knobloch et al., 2006) the contents of each are hereby incorporated by reference herein, in their entireties.

Methods of testing a potential therapeutic agent (e.g., a candidate drug, potential modulator, etc.) in an animal model are well known in the art. Thus potential therapeutic agents can be used to treat whole animals. The potential modulators can be administered by a variety of ways including topically, orally, subcutaneously, or intraperitoneally (such as by intraperitoneal injection) depending on the proposed use. Optimal dose will be empirically defined. Animals can be sacrificed by focused microwave beam irradiation, for example. These tests can be then be followed by human trials in clinical studies. Alternatively, in certain instances, human trials in clinical studies can be performed without animal testing.

Once a potential modulator/inhibitor is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential modulator may be synthesized de novo. The de novo synthesis of one or even a relatively small group of specific compounds is reasonable in the art of drug design. For all of the drug screening assays described herein further refinements to the structure of the drug will generally be necessary and can be made by the successive iterations of any and/or all of the steps provided by the particular drug screening assay.

Thus, in a still further aspect the present invention relates to a compound which could have been identified or was obtainable by the above-described method, wherein said compound hitherto has not been disclosed in the prior art as a drug for the treatment of a neurodegenerative, neurological or neuropsychiatric disorder, preferably wherein said disorder is associated with APP or a fragment thereof, more preferably wherein the disorder is selected from the group of memory impairment and learning disorders especially in the elderly, depression, Parkinson's disease, dyslexia, aging, cognitive decline, learning capabilities, intensity of brain waves, anxiety, concentration and attention, mood, general cognitive and mental well being, in particular of Alzheimer's disease.

Furthermore, the present invention relates to a composition for treating or diagnosing a neurodegenerative, neurological or neuropsychiatric disorder comprising the interacting molecule or a compound as described above and optionally a pharmaceutically acceptable carrier. Pharmaceutically acceptable carriers and administration routes can be taken from corresponding literature known to the person skilled in the art. The pharmaceutical compositions of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier. Further guidance regarding formulations that are suitable for various types of administration can be found in Remington's Pharmaceutical Sciences, Mace Publishing Company, Philadelphia, Pa., 17th ed. (1985) and corresponding updates. For a brief review of methods for drug delivery see Langer, Science 249 (1990), 1527-1533.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 ng to 10 mg units per day. If the regimen is a continuous infusion, it should also be in the range of 1 μg to 10 mg units per kilogram of body weight per minute, respectively. Progress can be monitored by periodic assessment. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as dopamine or psychopharmacologic drugs, depending on the intended use of the pharmaceutical composition. Furthermore, the pharmaceutical composition may also be formulated as a vaccine, for example, if the pharmaceutical composition of the invention comprises an anti-Aβ antibody for passive immunization.

In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The effective amount of a therapeutic composition to be given to a particular patient will depend on a variety of factors, several of which will be different from patient to patient. A competent clinician will be able to determine an effective amount of a therapeutic agent to administer to a patient to prevent or decrease ongoing disease. Dosage of the agent will depend on the treatment, route of administration, the nature of the therapeutics, sensitivity of the patient to the therapeutics, etc. Utilizing LDSO animal data, and other information, a clinician can determine the maximum safe dose for an individual, depending on the route of administration.

Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. a small organic compound may be taken for extended periods of time on a daily or semi-daily basis, while more selective agents, such as peptide mimetics or antibodies, may be administered for more defined time courses, e.g. one, two, three or more days, one or more weeks, one or more months, etc., taken daily, semi-daily, semi-weekly, weekly, etc.

Whereas the present invention includes the now standard (though fortunately infrequent) procedure of drilling a small hole in the skull to administer a drug of the present invention, in a preferred aspect, the agent/drug can cross the blood-brain barrier, which would allow for intravenous or oral administration. Many strategies are available for crossing the blood-brain barrier, including but not limited to, increasing the hydrophobic nature of a molecule; introducing the molecule as a conjugate to a carrier, such as transferrin, targeted to a receptor in the blood-brain barrier, or to docosahexaenoic acid etc. In another embodiment, the molecule can be administered intracranially or, more preferably, intraventricularly. In another embodiment, osmotic disruption of the blood-brain barrier can be used to effect delivery of agent to the brain (Nilayer et al., Proc. Natl. Acad. Sci. USA 92 (1995), 9829-9833). In yet another embodiment, an agent can be administered in a liposome targeted to the blood-brain barrier. Administration of pharmaceutical agents in liposomes is known (see Langer, Science 249 (1990), 1527-1533; Treat et al., in Liposomes in the Therapy of Infectious Disease and Cancer, Lopez-Berestein and Fidler (eds.), Liss, New York (1989), 353-365; Lopez-Berestein, ibid., 317-327; see generally ibid.). Comparison of the ability of histamine H2 receptor antagonists to cross the blood-brain barrier suggests that brain penetration may increase with decreasing over-all hydrogen binding ability of a compound (Young et al., supra). Begley et al. (J. Neurochem. 55 (1990), 1221-1230), herein incorporated by reference in its entirety, have more recently examined the ability of cyclosporin A to cross the blood-brain barrier. Methodology as used by Begley et al. includes: (1) measuring the brain uptake index (BUD with the equation for a tritiated agent compound: BUI=[(brain³H/brain¹⁴C)/(injectate³H/injectate¹⁴C)]*100, where the ¹⁴C reference compound is ¹⁴C butanol or an analogous solvent; (2) Brain perfusion studies; (3) Intravenous bolus injection studies; and (4) Studies with cultured cerebral capillary endothelium. All of such methods are envisioned in the present invention.

Hence, the present invention provides means and methods for drug discovery and development, in particular for drugs useful in the treatment and prevention of neurodegenerative, neurological or neuropsychiatric disorders such as Alzheimer's disease, which preferably rescue and/or confer resistance to disorders mediated either directly or indirectly by APP or fragments thereof. Accordingly, in a further aspect the present invention relates to a method for treating a neurodegenerative, neurological or neuropsychiatric disorder in a subject comprising administering to the subject an agent, wherein said agent
(i) binds to a protein selected from the group consisting of the proteins referred to in Tables 1, 2, 4, 5, 14, 15 and the corresponding human orthologs, paralogs or homologs thereof; or
(ii) binds to APP and is derived from a protein as defined in (i); wherein such binding results in the inhibition of functions or processing patterns that contribute to central nervous system disease, including APP turnover and amyloidogenic processing, cellular trafficking, signaling, degradation, isomerization, modification and direct or indirect regulation by APP of downstream processes like neuronal survival, synaptic plasticity, trafficking of growth factors, glucose metabolism among others.

Preferably, said agent can cross the blood brain barrier.

The present invention also provides a pharmaceutical pack or kit comprising one or more containers filled with one or more of the ingredients of the pharmaceutical compositions of the present invention. Associated with such container(s) can be a notice in the form prescribed by a governmental agency regulating the manufacture, use or sale of pharmaceuticals or biological products, which notice reflects approval by the agency of manufacture, use or sale for human administration. The composition, i.e. pharmaceutical composition of the present invention is of course particularly suitable for the diagnosis, prevention and treatment of amyloidosis, and in particular applicable for the treatment of Alzheimer's disease (AD).

As referenced above and demonstrated in accordance with the experiments performed within the scope of the present invention, a non-human transgenic animal has been generated, which is particularly suitable for identifying molecules, especially proteins capable of interacting with APP. In this context, it is prudent to stipulate that the concept behind the design of these non-human transgenic animals may be applied to research of neurodegenerative, neurological or neuropsychiatric disorders in general as well. Therefore, in a further aspect, the present invention relates to a non-human transgenic animal comprising preferably stably integrated into its genome, a foreign nucleic acid molecule encoding a protein involved in the onset or development of a neurodegenerative, neurological or neuropsychiatric disorder containing a tag, preferably operably linked to expression control sequences allowing transcription and expression of the nucleic acid molecule in the brain and/or CNS of the animal.

Examples of such genetically altered non-human animals showing neuropathological features and/or showing reduced symptoms are disclosed in the present invention; see the Examples and Figures. Strategies and techniques for the generation and construction of transgenic and/or knockout animals are known to those of ordinary skill in the art; see e.g. Capecchi, Science 244 (1989), 1288-1292; Hogan et al., Manipulating the Mouse Embryo: A Laboratory Manual, Cold Spring Harbor Laboratory Press, Cold Spring Harbor, N.Y., 1994; and Jackson and Abbott, Mouse Genetics and Transgenics: A Practical Approach, Oxford University Press, Oxford, England, 1999) and are described in detail in the present invention; see Examples and Figures.

It is thus within the scope of the present invention to provide a transgenic non-human animal that expresses, and in some embodiments overexpresses, a neurological disorder-associated protein as referenced above containing a tag such as SBP used in the present Examples. An exemplary and preferred transgenic animal is a transgenic mouse. Techniques for the preparation of transgenic animals are known in the art. Exemplary techniques are described in U.S. Pat. Nos. 5,489,742 (transgenic rats); 4,736,866; 5,550,316; 5,614,396; 5,625,125; and 5,648,061 (transgenic mice); 5,573,933 (transgenic pigs); 5,162,215 (transgenic avian species) and 5,741,957 (transgenic bovine species), the entire contents of each of which are herein incorporated by reference.

Furthermore, a number of transgenic non-human animal models of Alzheimer's disease have been described in the literature, the techniques and means such as vector constructs including an appropriate promoter described therein may be used for the, for example, brain-specific expression of a neurological disorder-associated protein in a non-human animal in accordance with the present invention. For example, U.S. Pat. No. 7,060,870 describes a transgenic non-human animal which has been genetically engineered to express amyloid-beta peptide alcohol dehydrogenase (ABAD) as well as human amyloid precursor protein hAPP695, hAPP751 and hAPP770 bearing mutations linked to familiar Alzheimer's disease in humans under the control of a nerve tissue-specific promoter. Similarly, a transgenic non-human animal showing Alzheimer's disease pathology because of the expression of a mutant human beta amyloid precursor protein with Swedish double mutation and Indiana mutation simultaneously has been described in international application WO2006/004287. A transgenic mouse model for tau-pathology in Alzheimer's disease has been described in US patent application US 2006/015959 A. Other transgenic animal models probably useful in Alzheimer research and other neurological disorders are described in US patent applications US 2006/058369 and US 2006/053499. The mentioned international applications as well as US patents and US patent applications also describe the use of such transgenic non-human animals for screening and testing modulating agents, substances and therapeutic compounds for neurodegenerative disorders, which can be equally applied to the non-human animals contemplated by the present invention, which (over)express or are knocked out for a neurological disorder-associated protein as described hereinbefore and in the following description of the experiments.

Modeling Alzheimer's disease is most advanced in transgenic mice and thus a vast of literature may be found which report on corresponding transgenic mouse models and the relationship of the pathological symptoms shown in the mouse model with the clinical syndrome as encountered in humans; see, for example, McGowan et al., Trends Genet. 22 (2006), 281-289; Games et al., J. Alzheimer's Dis. 9 (2006), 133-149; Sankaranarayanan et al. Curr. Top. Med. Chem. 6 (2006), 609-627.

Thus, in a further aspect of the present invention, it is preferred to make use of such a recombinant, genetically altered non-human animal, transgenic or knockout animal, as an animal model for investigating neurodegenerative diseases, in particular Alzheimer's disease. Such an animal may be a test animal or an experimental animal useful for screening, testing and validating compounds, agents and modulators in the development of diagnostics and therapeutics to treat neurodegenerative diseases, in particular Alzheimer's disease.

Thus, the skilled person will recognize that the animal model provided by the present application is also suited to be used for testing and validating potential drugs, compositions and medicine in particular in so far they concern APP and its interaction with its ligand proteins. In general, the transgenic mouse model is prepared according to standard techniques and described more detailed in Example 7, infra.

Furthermore, the transgenic non-human animal provided by the present invention may be easily adapted to be used for investigating neurodegenerative, neurological or neuropsychiatric disorders. Said disorders comprise but are not limited to Alzheimer's disease, Cerebral Amyloid Angiopathy, hereditary cerebral hemorrhage with amyloidosis Dutch type, Down's syndrome, Pick's disease, HIV dementia, fronto-temporal dementia with parkinsonism (FTDP-17), progressive nucleic palsy, corticobasal degeneration, parkinsonism-dementia complex of Guam, and other tauopathies. Further conditions involving neurodegenerative processes are for instance Parkinson's disease, Huntington's disease, amyotrophic lateral sclerosis and other motor neuron diseases, cerebro-vascular dementia, multiple system atrophy, and mild cognitive impairment. However, in a preferred embodiment the disorder is Alzheimer's disease (AD), because of which the animal comprises a foreign nucleic acid molecule as described in detail below and in Example 7, encoding a protein associated with AD, such as APP, preferably human APP, more preferably human full length APP tagged with SBP. Preferably, the expression control sequences are selected from those described infra, employing a CMV promoter as a preferred expression control sequence. In this context it is to be understood that the person skilled in the art will easily recognize that any other promoter as well as further enhancer elements may be suitable for the preparation of an appropriate vector construct comprising for example a nucleic acid molecule as described above. With respect to the tag used it is referred to the detailed description of the invention below.

The non-human transgenic animal of the present invention is particularly useful for performing the methods described above. Preferably, the non-human transgenic animal is a rodent, more preferably a mouse and most preferably the APP-TAP-AICD mouse described in more detail below.

The present invention also relates to a cell or tissue sample derived from the transgenic non-human animal as described above, preferably derived from the brain or CNS.

Since the transgenic non-human animal of the present invention is suitable to identify agents that alter or modify the interaction between the APP and its interacting molecule as described supra, the animal can be used for drug-screening relevant for the above-mentioned neurodegenerative, neurological or neuropsychiatric disorders, in particular neurodegenerative diseases or for diagnosing such diseases or for research purposes and the like. Thus, in a further aspect the present invention relates to the use of the transgenic non-human animal or the cell or tissue sample derived from that non-human animal as described supra for the screening of a drug useful in the treatment of a neurodegenerative, neurological or neuropsychiatric disorder, e.g., CNS disease, preferably a neurodegenerative disease, including but not limited to Alzheimer's disease, Parkinson's disease and the like, most preferably Alzheimer's disease or for diagnosing of or research for any of these disorders.

Screening microarrays allow for drug screening and can be applied in context with the present invention as well. Of course, as will be known to the person skilled in the art from publicly available literature, there are several applications for the use of microarrays which are enclosed herein by reference as far as they concern the use of any agent, complex, compound, composition or interacting molecule obtained by the methods of the present invention. The preparation of microarrays is described in for example international application WO2004/083818 and can be adapted according to the teaching of the present invention. Thus, in a further embodiment the present invention concerns a microarray comprising at least one complex and/or interacting molecule obtainable by the method of the present invention and defined above or a corresponding encoding nucleic acid molecule.

Furthermore, the present invention relates to the use of the complex or interacting molecule as described supra as diagnostic marker for a neurodegenerative, neurological or neuropsychiatric disorder as defined hereinbefore. Hence, the present invention also relates to a method of diagnosis for identifying a neurological disorder in a subject, comprising determining within a sample of said subject the protein and/or RNA level of one or more of the above-referenced neurological disorder-associated proteins which have been identified to interact with APP. Preparing appropriate specific detection means for determining the protein and/or RNA level(s) of one or more of the above-mentioned proteins are well within the skill of the skilled artisan and are described in the pertinent literature; see, for example, international application WO2006/002563 and the references cited therein. In one particular preferred embodiment, gene microarray technique may be used in order to analyze the expression of the corresponding genes. For example, oligonucleotide arrays may be used similarly as described in Jee et al., Neurochem. Res. 31 (2006), 1035-1044, with the adaptation that contrary to the microarray used in this publication the oligonucleotides for loading of the microarray in accordance with the present invention are predetermined to correspond to RNA and cDNA, respectively, encoding the above-mentioned proteins identified to be capable of interacting with APP as well as other neurological disorder-associated proteins to be identified with the screening methods of the present invention described herein. In an alternative embodiment, a protein- or antibody-based array may be used, which is for example loaded with either antigens derived from the mentioned neurological disorder-associated proteins in order to detect autoantibodies which may be present in patients suffering from a neurological disorder, in particular Alzheimer's disease, or with antibodies or equivalent antigen-binding molecules which specifically recognize any one of those proteins. For example, antigen microarray profiling of autoantibodies in rheumatoid arthritis has been reported by Hueber et al., Arthritis Rheum. 52 (2005), 2645-2655. Design of microarray immunoassays is summarized in Kusnezow et al., Mol. Cell. Proteomics 5 (2006), 1681-1696.

Accordingly, the present invention also relates to microarrays loaded with antigens of or antibodies specific for one or more of the neurological disorder-associated proteins identified in accordance with the present invention. Preferably, at least 5, more preferably, at least 10, most preferably 20, in particular preferred 25 antigen or antibody species are present on the array. Of course, in one embodiment, the microarrray of the present invention may contain almost all proteins that have been described herein to be associated with a neurological disorder, in particular Alzheimer's disease and amyloidosis, respectively. In a particularly preferred embodiment, the microarrays of the present invention represent substantially all of those neurological disorder-associated proteins which have been described in more detail in the preceding description such as profilin.

General methods in molecular and cellular biochemistry useful for diagnostic purposes can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996). Reagents, detection means and kits for diagnostic purposes are available from commercial vendors such as Pharmacia Diagnostics, Amersham, BioRad, Stratagene, Invitrogen, and Sigma-Aldrich as well as from the sources given any one of the references cited herein, in particular patent literature.

The present invention also relates to a kit for use in any one of the methods as described above, i.e. for identifying, isolating, determining and/or using the interacting molecules, agents, compounds, or composition of the present invention, said kits containing specific reagents such as those described hereinbefore further comprising for example selectable markers, reference samples, microarrays, culture vessels, and maybe some monitoring means. The kit preferably comprises at least one of the afore-mentioned molecules, as well as reference molecules for indicating the potential drug efficacy of an added agent, wherein the reagents are preferably kept in single containers. The kit of the present invention is preferably suitable for commercial manufacture and scale and can still further include appropriate standards, positive and negative controls. It preferably comprises at least one reagent which is selected from the group consisting of reagents that selectively detect the presence or absence of APP transcription products or translation products of an APP gene, and/or a processed or fragmented peptide of the translation product.

Preferably, the kit further comprises means for detecting a level, i.e. a decrease or increase of complex formation between APP and its at least one interacting molecule or an increased or decreased binding capacity compared to a control by, for example, labels comprising fluorescent label, phosphorescent label, radioactive label, which are known to those skilled in the art. Furthermore, the kit may comprise amyloid precursor protein APP as a substrate so that the generation of Aβ/amyloidogenic peptides as processing products can be measured as a result of for example binding of APP to processing enzymes such as secretases for example α-, β-, γ-secretases.

In addition, or alternatively, the kit of the present invention contains nucleic acid and/or protein/antibody based probes for the detection of any one of the above described APP interacting molecules and neurodegenerative, neurological or neuropsychiatric disorder-associated proteins, respectively.

Most preferably, the present invention relates to a kit useful for performing the methods of the present invention, said kit comprising an APP or a fragment thereof, comprising a tag such as defined in the description, or a recombinant nucleic acid molecule encoding such APP or fragment, a purification device, preferably a column suitable for performing purifications, in particular the purification step (b) as defined in the method, a control APP interacting molecule or a recombinant nucleic acid molecule encoding said control molecule, reagents for performing the methods of the present invention, a suitable detection means, spectroscopic devices and/or monitoring systems capable of monitoring complex formation of tagged APP with an interacting molecule (optionally further comprising instructions on how to perform any method of the present invention) as described supra.

Such kit would further typically comprise a compartmentalized carrier suitable to hold in close confinement at least one container and the compounds of the kit may be sterile, where appropriate. The kit may further include a transfer means, such as pipes for transferring the reagents or cells. In other embodiments, there may be components for application of agents, compounds or compositions to an individual, preferably an animal, such as a syringe, a needle, and so forth. The kit may further comprise components for extracting for example cells from a tissue of interest.

Furthermore, instructions can be provided to detail the use of the components of the kit, such as written instructions, video presentations, or instructions in a format that can be opened on a computer, e.g. a diskette or CD-ROM disk. These instructions indicate, for example, how to use the cell, agent, compound, composition and the like to screen test agents of interest. Most preferably, the instructions refer to the use of the kits in the methods concerning the identification and/or isolation of interacting molecules of APP or validation or assessment of potential drugs, agents, compositions or compounds influencing, either inhibiting or enhancing said interaction.

As already discussed in the context of the assay system of the present invention for screening putative drugs, the observations made in accordance with the present invention can also be applied to establish a novel method of identifying putative target genes for therapeutic intervention within the treatment of a given disease.

Furthermore, the invention relates to a method of identifying and obtaining an APP binding protein, compounds interfering with such binding, transgenic animals and vectors for generating the same, binding proteins and interfering molecules obtained according to the methods of the present invention.

Furthermore, instead of developing the identified drug in house, further drug development can also be achieved by a different company. Thus, in a further aspect the present invention relates to a method of conducting a drug development business comprising licensing, to a third party, the rights for further drug development and/or sales for drugs identified or profiled, or analogs thereof. For suitable lead compounds that have been provided, profiling of the agent, or analogs thereof, can be carried out for assessing efficacy and toxicity in animals, depending on the modalities of the agreement with the respective third party. Further development of those compounds for use in humans or for veterinary uses will then be conducted by the third party. The subject business method will usually involve either the sale or licensing of the rights to develop said compound but may also be conducted as a service, offered to drug developing companies for a fee.

### Alzheimer Dementia: Epidemiology, Symptoms and Diagnosis

Accounting for over 50% of cases, Alzheimer's Disease (AD) is the most common form of dementia (Bachman et al. 1992), afflicting 1% of 65-69 year olds with anterograde amnesia, with the prevalence doubling every five years, accelerating and reaching over 20% for those above 85. Cumulated risk throughout life for developing AD is 6.3% and 12% for 65-year old men and women, respectively. The difference is mainly, but not exclusively due to the greater life expectancy of women (Seshadri et al. 1997). Two different types of AD can be discerned, sporadic or late onset and familial or early onset AD (FAD), which constitute approximately 95% and 5% of all AD cases, respectively. While there are many risk factors contributing to sporadic AD, several are disputed and none of them is causative, contrasted to clear-cut mutations directly resulting in FAD, the discovery of which has been instrumental in defining the amyloid cascade hypothesis that explains important aspects of AD etiology. The difference in the two forms of the disease is mainly one of time course and severity, with nearly all FAD patients showing symptoms before age 65 and more rapid disease progression. Often, elderly people transit into AD through a period called mild cognitive impairment (Hanninen et al. 1995), where one of the primary cognitive symptoms of AD, failing memory, already manifests.

In AD, ailment is not limited to memory dysfunction, but encompasses, to various degrees of severity, aphasia, impairments in visuo-spatial processes, higher cognitive functions and behavioral deficits such as depression (McKhann et al. 1984). Importantly, this combination of afflictions progressively results in loss of ability to live without constant care, let alone independently. However, memory loss is typically the symptom that is most rapidly recognized by patient and family (Grober and Kawas 1997) and can also be assessed in verbal memory based word recall tests where the ability to encode new information is examined, with a good degree of specificity for AD (Knopman and Ryberg 1989).

Besides such and more detailed (DMS and NINCDS-ADRDA, (Widiger and Samuel 2005) (McKhann et al. 1984)) neuropsychological criteria, there are other methods for diagnosing AD with varying degrees of specificity and sensitivity. Risk factor genotyping can by definition only be a complementary measure in diagnostics, as alleles such as the ApoE gene ε4 variant are not causative. While biomarker concentrations in blood plasma are exceedingly low, recent studies searching in cerebrospinal fluid (CSF) from lumbar punctures for biomarkers upregulated in AD patients show the potential for discerning AD from other forms of dementia (Gretener 2005). As detailed below, one of the molecular hallmarks of AD is the formation of amyloid plaques, which can be visualized using β-pleated sheet specific contrast reagents (Higuchi et al. 2005), non-invasively as in magnetic resonance imaging, or dyes such as Thioflavin S or Congo Red that stain plaques in post-mortem brains. This latter histopathological examination, together with stainings for the second hallmark, neurofibrillary tangles, remains the definitive proof of AD, according to common consent.

### Neuropathological Changes in AD

### Amyloid Plaques

Parenchymal and vascular amyloid plaques are the molecular hallmark of AD, with the vast majority of therapeutic approaches tackling the problem of reducing Aβ formation and deposition from different angles. The term amyloid refers to fibrillar deposition of insoluble proteins in a non-native configuration. Plaque morphologies have been studied in great detail, but in the present application two main forms will be discerned—diffuse plaques and dense core plaques. As the former can be present in great numbers in healthy elderly who generally lack dystrophic neurites (Delaere et al. 1990), it is the latter that has generated most interest. The major component of these plaques are variously modified forms of Aβ, typically a 40-42 amino acid (aa) hydrophobic peptide derived from the Amyloid Precursor Protein as will be described in detail below, aggregated in fibrillar, β-pleated sheet form that, when stained by Thioflavin S or Congo Red can make up cross-sectional cortical loads of up to 25% (Cummings et al. 1996). Several other proteinaceous and metabolical secondary components of these plaques have been identified, most importantly however, they are additionally populated by activated microglia that are constantly involved in clearing Aβ (McGeer et al. 1993), which results in a state of constant inflammation.

### Neurofibrillary Pathology

Paired helical filament depositions of hyperphosphorylated forms of the microtubule associated protein Tau as neurofibrillary tangles (NFT) in cell soma, dendrites and as a component of neuritic plaques constitute a further hallmark of the disease at the ultrastructural level (Iqbal et al. 2005). Tau is a 50-70 kDa protein that stabilizes microtubules and promotes Tubulin polymerization under normal conditions, is however hyperphosphorylated by kinase/phosphatase imbalances (Grundke-Iqbal et al. 1986) resulting in lower binding to microtubules and aggregation in the soma (Gotz 2001). It also entails disruption of cytoskeletal structure and intracellular transport, which may be one reason for cell death (Price and Sisodia 1998), resulting in “ghost” tangles, or extracellular insoluble NFT deposits. Tauopathies can also occur independently of Aβ-plaque formation in diseases such as Pick's disease and frontotemporal dementia with Parkinsonism. This separation from Aβ-plaque formation is also demonstrated by the fact that neurofibrillary pathology is independently distributed from Aβ deposits—limbic and association cortices are affected first, primary cortices later, which is also used to define stages of the disease (Braak and Braak 1995).

### Brain Atrophy and Synaptic Dysfunction

On a macroscopic level, brain weight reductions, as defined by the changing percentage of brain volume inside the cranial cavity, are clearly visible and present in almost all AD cases, albeit more severely in FAD. Compared with control subjects, decrease in weight is 41% for the temporal lobe, 30% for the parietal lobe and 14% for the frontal lobe (Najlerahim and Bowen 1988). Further, it has been reported that while thickness reductions are moderate, cortical ribbon length is decreased in AD, resulting in loss of neuronal columns and correlating with cognitive deficiencies (Duyckaerts et al. 1985).

Vulnerability to AD-related abnormal protein depositions is highest among hippocampal neuron populations, with mainly the CA1 region being affected.

Further, synaptic morphology has been shown to differ between healthy controls and patients (Cotman and Anderson 1995). Concommittantly, presynaptic markers have been shown to be downregulated in AD patients, with vesicular components more strongly so than presynaptic membrane components (Shimohama et al. 1997).

### Microglia and Astrocyte Reaction

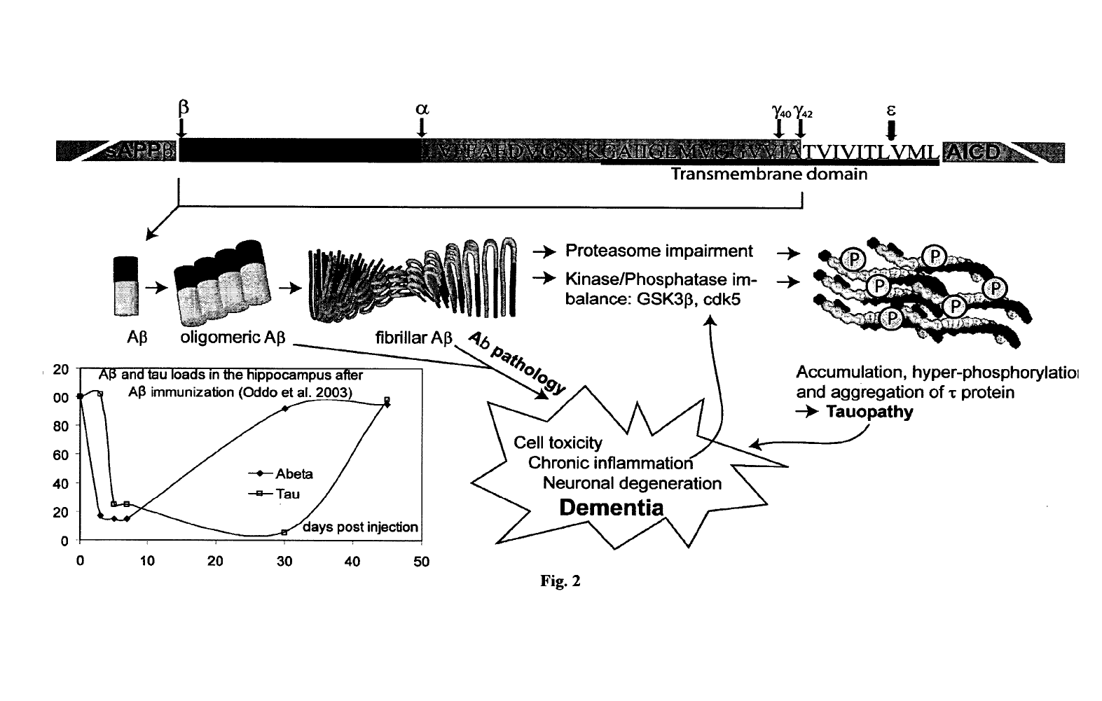

The definitive cause for the vast neuronal cell death in AD has not been determined, but is presumed to be due to a mixture of Aβ-mediated toxicity and apoptosis (Singer and Dewji 2006) and chronic inflammation (Cotman and Anderson 1995). Activated microglia and reactive astrocytes are found around plaques and release proinflammatory cytokines that can also have an effect on Tau pathology; see also FIG. 2.

### Genetic Causes of AD and Other Risk Factors

### The Amyloid Precursor Protein (APP) and Mutations Affecting Aβ Production

Early onset AD (FAD), the particularly aggressive form of AD running in families, has been found to be autosomally dominantly transmitted. In a landmark discovery, the APP gene, located on chromosome 21, was identified by linkage analysis (Tanzi et al. 1987). Albeit APP mutations only make up a small part of all AD patients, this seminal finding linked Aβ, the major component of amyloid plaques, to a gene that, when mutated, causes AD with almost total penetration. Interestingly, all the disease-causing mutations found hitherto in APP are localized to the Aβ domain or its boundaries. Besides mutations modifying the rate of Aβ aggregation (e.g. the Arctic mutation), they raise the amount of Aβ produced. This dosage effect (Rovelet-Lecrux et al. 2006) also holds true for Down Syndrome patients, whose genome contains a third chromosome 21, as they tend to develop amyloid pathology as soon as in the third decade of life (Mann et al. 1986).

It was found that two additional genes, Presenilin 1 and 2 (PS1, PS2), can also harbor mutations that infallibly result in FAD. Both are involved in generating the C-terminus of Aβ, and certain PS1 mutations, especially, result in most aggressive forms of premature AD.

### Risk Factors

For sporadic or late onset AD, many genes have been proposed to have an influence on disease outbreak probability. However, although only accounting for 10% of the predicted total genetic contribution to sporadic AD in the elderly, the ε4 allele of the ApoE gene is the only reported association that has been consistently replicated (Strittmatter et al. 1993). Humans homozygous for this allele have an 8 fold higher chance of developing sporadic AD, but even though the ApoE glycoprotein is a component and mediator of plaques (Bales et al. 1997), it is not causative or required for AD development (Puglielli et al. 2003).

Environment, nutrition and chronic medication have of course also been scrutinized for an effect of life-style on the risk of developing AD. With the possible exceptions of physical activity and enriched environments preventing plaque formation in mice (Lazarov et al. 2005), as well as cholesterol involvement in Aβ formation (Puglielli et al. 2003), there seems to be only one definitive major risk factor: age itself, with the percentage of centenarians with AD higher than 60% (Asada et al. 1996; Ravaglia et al. 1999).

To date, large ethnic differences in susceptibility have not been shown, albeit this may be due to the study population sizes being far larger in developed countries.

### The Amyloid Precursor Protein

### APP Discovery and Structure

Aβ, the main component of amyloid plaques, was isolated and its sequence determined by N-terminal protein sequencing; subsequently, the complementary full-length cDNA was cloned and sequenced, yielding a protein of 695 aa—far longer than anticipated—that bore resemblance to a cell surface receptor (Kang et al. 1987). This surprising finding led to the assumption that Aβ was derived from this “Amyloid Precursor Protein” by what was presumed to be aberrant catabolic processing. Kang and colleagues found the gene localized on chromosome 21, which fit well with the fact that trisomy 21, i.e. Down's syndrome, patients show AD-like pathology during their thirties.

Subsequent research showed that APP can be spliced in three different ways, yielding lengths ranging from 695 aa for the predominantly neuronal isoform to 751 aa and 770 aa for the longer variants that are also expressed in non-neuronal tissue and contain an additional so-called Kunitz-type protease inhibitor domain.

APP has a 590-680 aa extracellular N-terminal domain, depending on the splice isoform, which begins with a signal peptide that directs sorting. Except for the Aβ region, which is unique to APP, it has high homology with two other proteins termed APLP1 and APLP2 (Bauer et al. 1991; Wasco et al. 1993). Knockout mice lacking APP remain fertile and viable, only showing spurious evidence for defects (Zheng et al. 1995), but double knockout APP (−/−)/APLP2 (−/−) mice do not survive postnatality for long, while APP (−/−)/APLP1 (−/−) mice do (Heber et al. 2000). The physiological function of APP remains a topic of ongoing research, complicated by the functional redundancy conferred by its family members.

### APP Sorting and Processing

APP contains a signal peptide that targets it to the membrane of the endoplasmic reticulum (ER, cf. FIG. 3-12), which is cleaved after cotranslational insertion. Trafficked through the constitutive secretory pathway, APP undergoes various posttranslational modifications, including N- and O-linked glycosylation during its passage through the ER and Golgi, as well as phosphorylation (Weidemann et al. 1989).

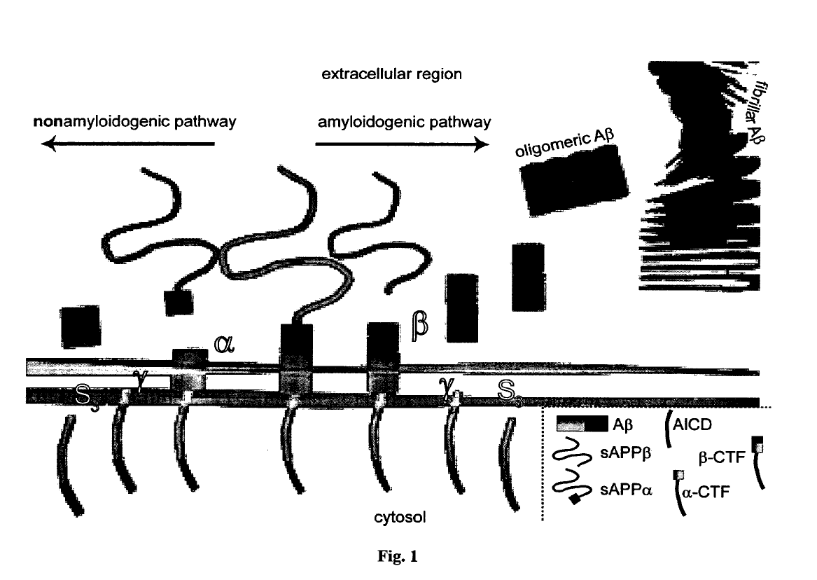

The most important events in the functional cycle of APP occur afterwards, as processing is shunted to either a normal, non-amyloidogenic pathway or an amyloidogenic, pathogenic pathway (Hardy 1997): either α-secretase cleaves APP, splitting the Aβ domain and rendering it harmless, or APP is cleaved by β-secretase, forming the N-terminal end of Aβ. In both cases, this ectodomain shedding is followed by intramembraneous γ-secretase cleavage, which releases the remaining extracellular fragment and the intracellular domain. Several factors regulate this important shunt and the workings of the responsible secretases, which will be described in the following and a schematic representation of which is depicted in FIG. 1.

### α-Secretase

Several different proteins from the ADAM (a disintegrin and metalloprotease) family of proteases have been shown to cleave APP inside the Aβ region, more precisely at the position Lysine 612/Leucine 613, cutting through the hydrophobic Aβ peptide. Not only does this cleavage prevent formation of Aβ, but it also results in release of sAPPα (secreted α-secretase derived APP fragment), to which neuroprotective properties warding off excitotoxicity to hippocampal and cortical neurons have been assigned (Mattson et al. 1993).

ADAMs are a widely expressed family of transmembrane proteins involved in integrin binding and thus cell-matrix interactions. Two α-secretases have been identified to date, TACE (TNF-α-converting enzyme) and ADAM10. The first is involved in a cleavage that is regulatable by protein kinase C (Buxbaum et al. 1993; Buxbaum et al. 1998), while the second has been extensively tested in APP transgenic mouse models, showing it to be responsible for both constitutive and regulated cleavage and to result in reduced plaque formation when overexpressed, and more and larger plaques when present in a dominant negative mutant form (Postina et al. 2004).

### β-Secretase

The type I membrane aspartyl protease BACE1 (β-site APP cleaving enzyme) was found to cleave APP at the N-terminus of Aβ, or Methionine 596 according to APP 695 nomenclature (Vassar et al. 1999). In cell culture, BACE1 was shown to be present mainly in late Golgi, and in smaller amounts in endosomes and plasma membrane (Yan et al. 2001), where it interacts with APP and cleaves it optimally at acidic pH such as encountered in endosomes after co-endocytosis of APP and BACE1 (Vassar et al. 1999). Although BACE1 has a homologue, BACE2 is far less strongly expressed in the brain and thus probably also not of significance for AD. BACE1 affinity for APP is far lower than that of α-secretases for APP, resulting typically in APP following the non-amyloidogenic pathway. This affinity and thus also the processivity of BACE1 is drastically increased for the FAD Swedish double mutation at the β-cleavage site of APP (Cai et al. 2001), with fatal consequences.

While BACE seems like an ideal drug target, as inhibition would directly reduce the production of Aβ, screening for small molecule inhibitors has proved very difficult due to the large active site of the enzyme.

### γ-Secretase

The final step in processing of APP occurs by γ-secretase, involving a two-step cleavage of the transmembrane region of APP by a multimeric protein complex. These characteristics, which are not typical for proteases, which typically cleave inside an aqueous environment, merit some attention:

Two different Presenilins, PS1 and PS2, were found to be linked with processing of APP, and to be localized mainly to the Golgi apparatus and to some extent to the plasma membrane (Annaert et al. 1999; Ray et al. 1999). They are both proteins of approximately 50 kDa that contain their active site, aspartyl residues 257 and 385, within transmembrane domains 6 and 7. Functional value was assigned to these residues by site-directed mutagenesis showing mutagenization of either of the two residues to abolish activity (Kimberly et al. 2000).

The importance of γ-secretase for the production of Aβ was demonstrated in cell culture systems derived from PS1 (−/−) and PS2 (−/−) knockout mice overexpressing APP; Aβ production and secretion in these cells was strongly reduced (De Strooper et al. 1998). This data complements the fact that many FAD patients suffer from PS1 mutations resulting in a gain of function.

Presenilins undergo autocatalytic cleavage required for formation of an activated heterodimer (Levitan et al. 2001). Further, glycerol gradient centrifugation showed the resulting PS fragments to cofractionate at high apparent molecular weights up to 150 kDa (Capell et al. 1998), suggesting that PS exist inside a larger complex under physiological conditions. Following the identification of individual additional components, a seminal study reconstituting γ-secretase activity in yeast showed the proteins PS1, Nicastrin, Aph-1 and Pen-2 to associate in stoichiometric ratios to generate a fully functional γ-secretase complex capable of processing APP (Edbauer et al. 2003).

As already mentioned above, the total amount of Aβ is only one aspect of the contribution of γ-secretase to AD pathology; whether the more hydrophobic and aggregation-prone Aβ₄₂ or the Aβ₄₀ variant is formed depends on the second intramembraneous processing step alluded to at the beginning of this chapter: recent evidence shows an additional PS-dependent ε-cleavage site closer to the intracellular leaflet of the phospholipid bilayer, at L645, to precede cleavage at the γ-site and determine which Aβ variant is produced (Funamoto et al. 2004).

This ε-site is homologous to the S3 site involved in Notch cleavage, important to development, which underlines the similarity between APP and Notch processing (Gu et al. 2001), with Notch and several other proteins also being substrates of γ-secretase cleavage. Commonly, it seems that ectodomain shedding is required prior to cleavage by γ-secretase and that the intracellular domain can translocate to the functionally relevant intracellular compartments (Ehrmann and Clausen 2004).

### The Amyloid Cascade Hypothesis

In search for a coherent connection between the two neuropathological hallmarks of Alzheimer Disease, Selkoe et al. and Hardy et al. developed the “amyloid cascade hypothesis”, which in spite of some controversy remains the pillar of AD research until today (Selkoe 1991; Hardy and Higgins 1992). In brief, it contests that the build-up of abnormal levels of Aβ, especially the more hydrophobic A342 (containing additionally Alanine and Threonine at the C-terminus) results in formation of Aβ oligomers of increasingly higher molecular weight (MW) and nucleation of amyloid plaques in brain parenchyma and along cerebral blood vessels, entailing the plethora of adverse effects that finally make up AD. Their compelling lines of reasoning were twofold: all known FAD mutations affected APP or its processing, underlining the central role of this protein in AD. Further, combining the fact that Tau is phosphorylated by Ca²⁺/Calmodulin-dependent kinase and that Aβ may increase intracellular Ca²⁺ levels (Hardy and Higgins 1992), they showed how APP pathology might also induce Tau pathology.

The hypothesis was challenged by data showing a bad correlation between amyloid plaque load alone and the severity of disease (Lue et al. 1999), and by the fact that APP-overexpressing mice don't show strong signs of neurodegeneration in spite of widespread amyloid deposition (Hsiao et al. 1996). This led to a modified amyloid cascade hypothesis which takes into account additional factors such as the toxicity of soluble Aβ species (Hardy and Selkoe 2002).

However, injection of aggregated Aβ₄₂ into mice transgenic for human Tau with the pathogenic P301L mutation resulted in a strong increase in the number of NFTs, suggesting a strong in vivo influence of Aβ fibrils on Tau pathology (Gotz et al. 2001). Additionally, recent evidence conveys Tau pathology to be a result of or at least dependent on amyloid pathology, with triple transgenic mice containing APPswe, a Presenilin mutation (M146V) and Tau P301L developing Aβ pathology prior to Tau pathology in spite of concomitant expression (Oddo et al. 2003).

Mechanistic and toxic effects of Aβ resulting in synaptic dysfunction and ultimately neuronal loss are still a matter of debate, with suggestions ranging from toxicity through raised H₂O₂ levels (Behl et al. 1994), disruption of Calcium homeostasis and raised exitotoxicity (Mattson et al. 1992) to formation of pores in the membrane of cells (Singer and Dewji 2006). Indisputable however is the evidence that Aβ and its deposition are responsible for chronic inflammation processes, with microglia activated around neuritic plaques (McGeer et al. 1993) and inflammatory cytokines detected in AD brains, which in turn can induce Tau kinases (Bauer et al. 1991).

### Search for a Physiological Function of APP

Hitherto, the focus of this introduction was on the direct relevance of APP to Aβ production and AD. As already mentioned above, the physiological role of APP has remained elusive and prompts further analysis. The following summary of findings focuses separately on the extracellular domain and on the intracellular domain, as they both convey different interactions and functions.

As all three isoforms of APP are produced at high levels in the brain, with APP₆₉₅ found mainly in neurons, but also together with the longer isoforms in microglia and astrocytes (Haass et al. 1991), APP and APLP knockout mouse brains were analyzed for brain-specific phenotypes. Single knock-out and APP (−/−)/APLP1 (−/−) mice show minor phenotypes, with no obvious brain histopathological abnormalities and even undiminished survival of cortical neurons (Heber et al. 2000). However, as soon as APLP2 is knocked out in addition, the mice die early postnatally, indicating that while APP and APLP2 mediate redundant functions, together they play an essential physiological role. Recent work demonstrates that mice lacking all three APP family members not only die shortly after work, but that they also develop a severe brain disorder mimicking symptoms of lissencephaly in 81% of cases (Herms et al. 2004), suggesting an important role of APP and its family members in normal brain development. In part, this was shown to be due to aberrant neuronal migration, which led researchers to look for links between APP and cytoskeleton or extracellular matrix (ECM) adhesion. One such analysis looked into conserved regions throughout the characterized inter-species APP members and identified several conserved adhesion and ECM interaction domains (Coulson et al. 2000). In sequential order beginning at the N-terminus, these include: a Heparin binding domain (BD), a Copper and a Zinc BD, a second Heparin BD, a Collagen BD and a Chondroitin sulfate attachment region which may bind Glycosaminoglycans.

Besides the N-terminal signal peptide which is cleaved after insertion of APP into the ER membrane, the Kunitz protease inhibitor domain has also been mentioned above, which is interspersed between the Zinc and second Heparin BD and which has been implicated in interfering with blood coagulation in vitro through inhibition of factor XIa (Smith et al. 1990). A large body of evidence has accumulated showing APP expression to be correlated both spatially and chronologically with synaptogenesis and neurite outgrowth (Loffler and Huber 1992; Ohta et al. 1993; Small et al. 1999).

### APP Intracellular Domain Functional Regions

In the 50 aa APP intracellular domain, henceforth AICD, three main sites of protein-protein interaction have been identified. In sequential order, these are the QYTS Basolateral Sorting Signal (BaSS), the G0-protein binding region and the YENPTY-sequence containing region at the extreme C-terminus.

The membrane-proximal QYTS sequence binds PAT-1 (protein interacting with APP tail 1), which has a Kinesin light chain (KLC) homology (Zheng et al. 1998). As KLC is involved in basolateral trafficking, this data seems to fit with earlier findings showing APP to be sorted accordingly in polarized cell culture systems (Haass et al. 1994; Zheng et al. 1998).

APP is a transmembrane protein with the intracellular domain showing strong conservation across vertebrate species. The group that discovered APP already stated it to bear resemblance to typical cell-surface receptors (Kang et al. 1987) and it came as no great surprise when evidence surfaced that the region encompassing residues H657 to K676 can bind and activate G0 protein (Nishimoto et al. 1993). G0 trimeric protein can activate K⁺ channels while inactivating Ca²⁺ channels, or activate phospholipase C. However, not much further data has been put forth showing this to be an important mechanism for cellular signaling, contrary to Notch-like signaling, with several publications pointing to this second, more central signaling pathway.

The final region, the YENPTY sequence, has proven to be the region where most protein-protein interactions take place (Borg et al. 1996; Russo et al. 1998). Importantly, as for PAT-1 binding to the QYTS sequence, phosphorylation of individual Threonine or Tyrosine residues can strongly shift binding preferences, an ideal prerequisite for signaling and providing regulation of Aβ production (Buxbaum et al. 1993; Ando et al. 2001). Further, this phosphorylatable region could interact with many phospho-Tyrosine binding domain (PTB) containing proteins. The NPTY-sequence is conserved in all APP family members, a further hint at its functional importance. For the above reasons, the YENPTY region has been the focus of intense research.

Yeast-Two-Hybrid Screens and their Legacy

The function of a protein can often be deduced from the nature of its interaction partners, according to the widely used “guilty by association” reasoning, whereby the function of two interaction partners can often be roughly correlated, and it was hoped that new functions of APP might be found in this way.

### The Yeast-Two-Hybrid System

The Yeast-two-hybrid (Y2H) system is a screening system for finding proteins that interact with each other. Classical transcription activators can be separated into a DNA binding (DBD) and a transcription activating (TA) domain that maintain their function if brought into close proximity by two proteins that interact with each other, one attached to each subdomain of the transcriptional activator. Y2H works as follows: A plasmid with the bait-protein fused to the DBD and a plasmid library containing cDNAs fused to the TA are mixed and co-transfected into yeast cells. When bait and cDNA derived proteins interact, the TA and DBD are reconstituted, and result in expression of a reporter protein.

The first Y2H study using the 47 C-terminal aa of AICD as bait appeared towards the end of 1996, describing 6 clones that represented two different PTB proteins and potential homologues thereof, Fe65 and X11 (McLoughlin and Miller 1996). These findings were confirmed nearly simultaneously by another group also showing Fe65 to interact with AICD (Bressler et al. 1996). Further Y2H studies demonstrated interactions with additional proteins (Matsuda et al. 2001; Scheinfeld et al. 2002).

A quick summary of important AICD/APP-interacting proteins may prove beneficial to the discussion of differences to and overlaps with the mass spectrometry (MS) results of the present invention.

### Proteins Involved in APP Sorting

Besides the BaSS which interacts with PAT-1, the YENPTY sequence can also function as a sorting signal by mediating Clathrin binding and coated pit internalization of APP (Guenette et al. 1999). This internalization seems important for two reasons: 1) BACE cleavage may occur predominantly in endosomes, based on its subcellular distribution and 2) recycling of APP in synaptic vesicles may be an important part of its life cycle (Marquez-Sterling et al. 1997).

### (i) Jip1

Jip1 was another protein repeatedly identified as an interactor of AICD in Y2H screens. Like APP and Fe65, it belongs to a family of proteins. The two members present in humans, JNK-interacting proteins 1 and 2 (Jip1 and Jip2), both have PTB domains that can interact with the YENPTY region and link APP to JNK, which in turn can phosphorylate AICD at Threonine-668 (Inomata et al. 2003). Importantly, a complex can be formed of Kinesin, Jip1 and APP that results in fast axonal transport of vesicles containing APP, BACE and Presenilin, which may be an important mechanism in Aβ production in axons and synapses (Kamal et al. 2001).

### (ii) X11

A further protein that interacts with AICD is X11, also named Mint, again a protein that belongs to a family of proteins, with X11α and X11β expressed in the brain and the ubiquitous X11γ. They contain a C-terminal PTB domain and two protein dimerization domains. Even though the mode of binding is independent of phosphorylation and thus slightly different to that of Fe65, X11 can also bind to the YENPTY domain (Borg et al. 1996). Through its PDZ domains, the X11s can interact with a variety of other proteins, among them the Presenilins (Lau et al. 2000). Most importantly in the connection with APP and AD, this interaction with Presenilin may be the basis for the wealth of evidence showing X11 to modify the processing of APP (Borg et al. 1998; Ho et al. 2002; King et al. 2003).

(iii) The Fe65 Network

Fe65 is part of a gene family together with two other proteins, Fe65L1 and Fe65L2, is expressed at high levels in neurons and was consistently found to interact with AICD in Y2H screens (Bressler et al. 1996). It contains two PTB domains and a so-called WW-domain. The C-terminal PTB₂ interacts with AICD, while the other two domains are free to interact with other proteins. Mena, the mammalian homolog of the Drosophila enabled gene, binds to the WW-domain, yielding a possible link between AICD and the cytoskeleton (Ermekova et al. 1997), as Mena in turn binds Profilin, which modulates actin polymerization. Depending on the cell-culture system used, there is discrepant evidence that Fe65 modulates APP processing and changes Aβ levels (Guenette et al. 1996; Ando et al. 2001). However, these publications do show APP and Fe65 to co-localize in the ER, Golgi and endosomes. As will be described below, Fe65 can form tripartite complexes with AICD and Tip60 by simultaneously binding AICD to PTB2, and Tip60 to PTB1.

Tip60 itself is a Histone acetyl transferase that enables transcription by modifying the Histone code to incorporate additional negative charges, resulting in local unwinding of chromatin due to Coulomb repulsion (Sterner and Berger 2000).

With Fe65, Jip1 and X11 all binding to the YENPTY region, the question of competition invariably was addressed (Lau et al. 2000; Inomata et al. 2003), showing this region to indeed be a site of balanced interactions that can for example be influenced by phosphorylation (Ando et al. 2001).

### RIP and Nuclear Signaling by AICD

Using a system similar to the Y2H screen AICD was shown to weakly induce a reporter gene when fused to the C-terminus of a Ga14 BD (Cao and Sudhof 2001). It was also shown that this mild transcriptional activity was drastically enhanced when cotransfecting two Y2H-screen derived partners of APP, Fe65 and Tip60. Jip1 was also shown to induce transcription together with AICD, even without requiring Tip60 and by a different mechanism (Scheinfeld et al. 2003). A later follow-up demonstrated AICD to be required to activate Fe65 for concomitant translocation into the nucleus (Cao and Sudhof 2004). Both NICD and AICD do not contain any DNA BD, which is why nuclear signaling necessarily depends on additional interaction partners. Confocal laser scanning microscopy (CLSM) experiments unequivocally revealed nuclear translocation of Fe65-AICD and the formation of a tripartite AICD/Tip60/Fe65 (AFT) complex in distinct nuclear structures ((Von Rotz et al. 2004).

Nuclear transcriptional activation by a proteolytically derived fragment of a transmembrane precursor is a relatively new concept in cell signaling but had been observed for other proteins prior to APP, most notably for Notch and the SREBP, and has been termed “regulated intramembrane proteolysis”, or RIP (Ebinu and Yankner 2002). Two versions are observed, classified depending on whether a single pass transmembrane protein or a multipass protein is cleaved (Rawson 2002). In both cases, the mechanism entails a two-step procedure, in which typically an ADAM protease first performs ectodomain shedding, followed by intra-membrane cleavage by another enzyme. For type I RIP, such as for Notch or APP, this second step is performed by Presenilins. This signaling mechanism is a trade-off between speed, as gene regulation involves directly a fragment derived from the activated receptor, and efficiency of signal amplification.

Even though RIP is not yet as well-researched as other cellular signaling mechanisms, the Presenilins are known to have a wide variety of substrates, many of which have an important physiological regulatory function: Notch (Geling et al. 2002), APP family members, LRP (May et al. 2002), N- and E-Cadherins (Marambaud et al. 2002), and others. This has important implications for treatment of AD; while γ-secretase is a more easily druggable target than β-secretase, with several inhibitors already in use for non-clinical purposes, blocking it to reduce Aβ-production could result in serious side effects. For example, zebra-fish treated with such an inhibitor showed a severe neurogenic phenotype (Geling et al. 2002), although of course such treatment occurred far earlier than it would in patients developing AD. Also, especially in the context of AD, AICD itself—production of which also is blocked by γ-secretase inhibitors—regulates production of Neprilysin, an Aβ degrading protein that is not produced in PS-knockout mice (Pardossi-Piquard et al. 2005).

### AD Treatment and Implications for AICD Signaling

### Immunotherapy

In 1999, a revolutionary strategy for AD-treatment was presented, breaking with the traditional view of the brain as an immune-privileged organ, when it was shown that active immunization with aggregated Aβ₄₂ in human—APP transgenic mice prevented plaque formation (Schenk et al. 1999). This led to a plethora of research followed by a first series of clinical trials that had to be aborted due to cases of encephalitis in 6% of patients. Nevertheless, patients that generated antibodies against beta amyloid plaques showed reduced cognitive decline (Hock et al. 2002).

### Conventional Drugs and Secretase Inhibitors

Conventional drugs are small molecular weight compounds and typically inhibitors of a certain type of membrane receptor or a specific enzyme. Currently, only two types of such drugs are FDA-approved: antagonists of NMDA-Receptors and cholinesterase inhibitors. Long-term potentiation (LTP), one of the fundamental mechanisms involved in learning and memory retention, depends on NMDA-Receptor function. Excessive activation of NMDA receptors leads to Ca²⁺ influx-induced cytotoxicity. There is evidence for Aβ-induced imbalances to this system through raised vulnerability to excitotoxicity (Mattson et al. 1992), which can be reduced by Memantine.

AD patients show reduced synthesis capacity for acetylcholine compared to age-matched controls, which may be linked to memory performance (Winkler et al. 1998). By inhibiting degradation of acetylcholine through its esterase, the neurotransmitter has a longer half-life in the synaptic cleft, which effectively stabilizes cholinergic signaling.

Additionally, research into non-steroidal anti-inflammatory drugs (NSAIDS) has been sparked by the discovery that some compounds of this class can modify the preferred cleavage position of γ-secretase, shifting the ratio of Aβ₄₂ vs. Aβ₄₀ towards the less aggregation-prone shorter variant, without reducing production of AICD (Weggen et al. 2003).

However, most hope lies with the development of β- or γ-secretase inhibitors. Advocates of the amyloid-cascade hypothesis are confident that reducing Aβ-production in this way would not be a symptomatic treatment but could actually be curative. However, there are two major setbacks: first, γ-secretase has many other substrates besides APP and generally blocking its activity may result in unwanted phenotypes, perhaps strongly limiting any therapeutic window. Secondly, β-secretase, for which such problems are not anticipated, has a very broad active site cleft, against which only oligopeptide inhibitors have been found, which cannot be used as drugs.

### Processing Modifiers

In summary, no ideal drug target has yet been found, and an interesting area of research in this regard are the APP-cleavage modifying or retarding effects of several known AICD interacting proteins (King and Scott Turner 2004) as well as the search for additional so-called second-site modifiers.

Overexpression of X11α in Hek 293 cells results in raised levels of full-length APP versus secreted APP or Aβ variants, with the specificity of the effect proven by mutations in the YENPTY region abolishing it (Borg et al. 1998). This modulation of APP cleavage may be influenced by trafficking (King et al. 2003) and interaction with Presenilins (Lau et al. 2000). Munc 18 a interacts synergistically with X11 to nearly totally reduce γ-secretase activity on APP, although the exact mechanism is unclear (Ho et al. 2002). Munc 18 a interacts with Syntaxin 1a as well as with the N-terminus of X11, which may play some role in this synergy. Like X11, Jip1 can stabilize APP processing and reduce the secretion of sAPP and Aβ in cell culture systems (Tarn et al. 2002). Here, specificity was shown by deletion of the PTB region in JIP responsible for interaction with APP, which eliminated processing inhibition.

All these effects show a reduction of APP-processing by gain of function manipulations. As activation of beneficial catalytic activity is a notoriously difficult approach in drug development, it would be of great interest to discover proteins that accelerate processing of APP through interaction with AICD, besides helping to define the physiological functions of APP. This was one of the thoughts in mind when designing the proteomics approach of the present invention to find new interaction partners of APP, as described in detail in the following.

### Technical Primer

The present invention focussed on the identification of APP interaction partners by mass spectrometry (MS). Although MS analysis is too large a field to be treated in great detail, however, the basic concepts of protein complex analysis by MS will be picked up at several points throughout the description of the present application. Therefore, it appears reasonable to attempt a brief introduction to core elements of the proteomics approach of the present invention to finding additional interaction partners of AICD and APP.

### Tandem MS

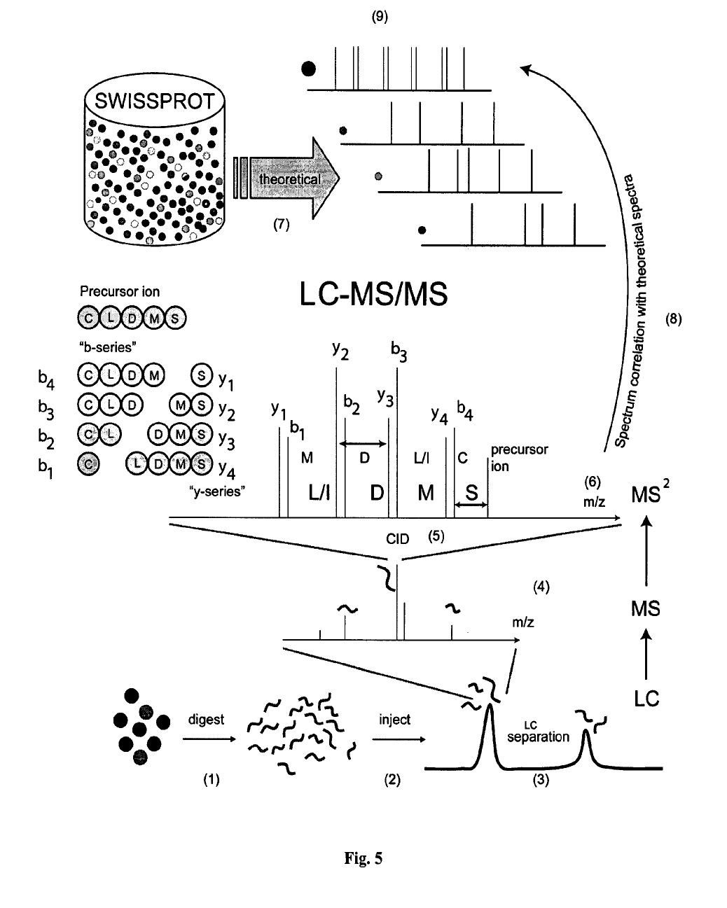

For the unbiased identification of proteins from complex mixtures, the dominant technique used until recently was to analyze proteins through peptide mass fingerprinting. Proteins were either purified to homogeneity or separated in one-dimensional or two-dimensional gels (1DGE/2DGE) depending on complexity of the sample. Excised gel bands or spots were trypsinized and typically measured by matrix assisted laser desorption-ionization time-of-flight analysis mass spectrometry (MALDI-TOF) and samples assigned to proteins from a database according to the degree of fit of the measured peptide masses in the sample and those from a theoretical digest. As soon as several proteins were present in the same sample, however, the obtained spectra were nearly impossible to deconvolute. For highly complex samples, however, even 2DGE, still the most powerful protein separation technique to date, can contain several proteins inside of one silver stained spot. The advent of tandem mass spectrometry radically changed the process of analyzing complex samples: as the name implies, two separate MS analyses are run sequentially, constituting one basic work cycle. Between the two, a crucial event occurs: individual peptide peaks from the first MS scan are selected and fragmented by collision induced dissociation (CID) with inert He gas molecules. Breaking of covalent bonds can occur at several positions, depending on the energies involved, but under typical conditions takes place inside the peptide bond, yielding two different statistically distributed series of peptide fragment ions, depending on whether the precursor ion charge is maintained on the N-terminal (b-series) or the C-terminal fragment (γ-series), which is then detected via the second mass analyzer (the 2^{nd} MS in “MS/MS”), as depicted in FIG. 5. Such a series can be theoretically calculated—albeit without intensity information—for all the proteins in a database and the spectra from the second MS scan can be compared to these theoretical ones, yielding a cross-correlation score as a measure of overlap and thus certainty of correct peptide identification.

To appreciate the second revolution in high-throughput proteomics, online separation of complex mixtures, a brief review of current apparatus' may be of advantage. Roughly, there are four main types of mass analyzers: quadrupoles, ion traps, Fourier-Transform ion cyclotron resonance (FT-ICR, or FT-MS for short) and time-of-flight (TOF). Ion traps and FT-MS both allow retention and analysis of selected peptides by precise application of radio-frequency electric fields to control ion orbits and can easily be coupled to online fluidic separation systems, as ionization of analytes occurs through electrospray ionization (ESI); volatile solvents evaporate from the pH-dependently charged peptides until the charge density is so high that equal-charge repulsion results in a so-called Coulombic explosion, which sets free finely dispersed charged peptides into the gas phase. LC-MS/MS equipment, notably the ThermoFinnigan LCQ-Deca has been effectively and repeatedly applied with great success to the analysis of protein networks (Ideker et al. 2001) and signaling complexes (Bouwmeester et al. 2004) and thus seemed appropriate to approach the unbiased analysis of the AICD intracellular holo-complex.

The classic mass analyzer is the TOF, basically measuring the time ions take to hit the detector after having been accelerated in an electric field. The acceleration and thus the time to detection straightforwardly depend on the mass/charge (m/z) ratio. Although recent quadrupole-TOF (QTOF) hybrid machines have made their appearance, TOF typically depends on ionization by MALDI, which requires crystallization of a peptide/laser-induced proton-transferring matrix mixture onto a metal plate, thus decoupling sample separation and MS analysis. With the advent of iTRAQ labeling and highly sensitive MALDI-TOF/TOF equipment, this ruptured process flow has been reinstated, but only at the cost of high sample preparation time.

### Quantitative Proteomic Techniques

The desire to compare protein levels between different samples, especially such as diseased vs non-diseased tissue has long been part of the search for biomarkers as tell-tale signs of disease. While 2DGE stains allow visual comparison of abundant protein levels, the transition from gel-based proteomics to in-solution proteomics brought the problem of quantitation by MS. Mass spectrometry is not an inherently quantitative method, as chromatography, ionization and other parameters cannot be entirely controlled between runs and with LC-MS/MS, there is the additional undersampling issue, meaning that a peptide that is identified in one run may just by chance go totally undetected in a second run.

Therefore, several labeling techniques were developed that allowed isotopic labeling of proteins in such a way that allows mixing the samples and analyzing them by MS at the same time. Cells can be grown in medium in which a certain amino acid contains a heavy isotope (Ong et al. 2002), albeit this without a doubt has a certain degree of influence on the biology of these cells. Alternatively, trypsinization of one sample can take place in heavy water (H₂O¹⁸), which labels the N-terminally located peptide with O¹⁸ (Yao et al. 2001). A pioneering event in the proteomics field was the introduction of isotope coded affinity tags (ICAT), which have the advantage of labeling Cysteine-containing peptides and simultaneously allowing selective pull-down thereof, which reduces sample complexity. On the downside, ICAT also reduces protein sequence coverage and results in a total miss of any proteins that do not contain Cysteine (Gygi et al. 1999). The newest addition to the available arsenal are the iTRAQ reagents that label all free amine groups, i.e. each tryptic peptide. Here, the crux is that they contain an isotopic balance that results in simultaneous elution from separation columns of two specifically labeled peptides from two different samples and only one peak in the full-scan MS spectrum. Peptides from both samples are therefore analyzed by CID at the same time.

As will be discussed later, at first glance it appeared as if the application of LC-MS/MS would be appropriate, since Western Blotting experiments gave the impression that it would be easy to separate proteins bound specifically to APP from background proteins. However, in fact the use of quantitative proteomics was necessary and therefore, the most conclusive MS data was finally derived from iTRAQ labeled samples.

### One Main Gists of the Present Invention

### Search for Additional Interaction Partners

The exact physiological role of APP is still not understood in detail. While several proteins are now known to interact with APP and AICD, there is no reason for assuming the list to be complete, especially as most studies used the same basic Y2H screening technology to find interaction partners. With the well-known interaction partners of AICD already under scrutiny by their discoverers, a different, proteomics—based approach was attempted to find additional proteins. To use such an approach seemed reasonable from several viewpoints: a) screening techniques based on different readout techniques are sure to be complementary, b) Y2H screens can not take place under physiological conditions or c) result in isolation of an entire holocomplex. This last point is essential and will be discussed later in view of the proteins identified, as it means that, contrary to Y2H screens, it should be possible to analyze by affinity purification and MS proteins that interact indirectly with APP through intermediates, as long as they are physically stably attached to the holocomplex.

### Study Mechanisms Affecting AICD Signaling

As RIP plays such an important role in APP processing, there are still further inquiries necessary to be answered in view of transcriptional regulation by AICD (Von Rotz et al. 2004) and in view of the fact that while most RIP cleavages by Presenilins are straightforward affairs, with only one pathway possible, APP ectodomain shedding can occur in a pathogenic and nonamyloidogenic fashion. This flexibility might seem unnecessary if one is not able to influence downstream signaling, especially as BACE and ADAM subcellular localization differs, which could influence ease of access of RIP-derived AICD to the nucleus. One main gist of the present invention was therefore to find out whether there are observable differences in nuclear signaling between the amyloidogenic and non-amyloidogenic pathways, which might also have implications for therapeutic treatments targeting this central amyloidogenic pathway.

### APP and AICD Interaction Partners: a Proteomics Approach

The field of proteomics is growing at a phenomenal pace, both technology- and result-wise, however, albeit an increasingly successful field, it is certainly not a homogenous one, with several different technologies vying for dominance in protein analytics, as described supra. Testimony to this fact is that different kinds of proteomics techniques, have been tried to solve the above problem.

The basic idea underlying the approach of the present invention was to test various approaches to isolating the AICD protein holocomplex and to then analyze the purified samples from in-solution digests by tandem mass spectrometry, as introduced supra, since there was no indication or incentive whether using an anti-body based IP approach or a synthetic bait peptide approach would yield the required results in combination with cell lysates or mouse brain homogenates. As will be described in the following, it turned out that to obtain best results, a more elaborate and time consuming methodology to identify proteins interacting with AICD had to be established.

Pull-Downs with Synthetic AICD

### Recombinant Bacterial Peptides

The basic idea was to use AICD as bait in form of an immobilized peptide to bind human cell lysates or mouse brain homogenate to pull down proteins interacting with AICD.

To this end, first the 51 C-terminal aa of AICD was cloned into the pET24 system from Novagen with either an N- or a C-terminal Hexahistidine tag (9.24 and 7.22 kDa, respectively). This system allows transgene production of up to 50% of cellular protein, using the T7 viral promoter (Studier and Moffatt 1986). One goal was to purify from E. coli, Biotinylate and immobilize with Streptavidin resins the peptides produced from either of these constructs, depending on yield and solubility. However, in both cases, yield was low and purification using the His-tag alone resulted in a solution with many additional protein bands as detected by Coomassie staining; see FIG. 3-1 of EP 06 025 239.2. Such a crude eluate could of course not be immobilized and used to specifically bind proteins interacting with AICD.

Therefore synthetic AICD peptides were purchased; however, before obtaining these, a Biotinylated AICD peptide resembling 45 of the last 50 aa of AICD (bioAICD) with the sequence LKKKQYTSIHHGVVEVDAAVTPEERHLSKMQQNGYENPTYKFFEQ (SEQ ID NO: 1) was used for initial experiments.

### Ciphergen

Using a Ciphergen surface-enhanced laser desorption/ionization time-of-flight MS apparatus (SELDI-TOF), it was intermediately attempted to set up an analytical method with this peptide to observe differences between the pull-down experiments performed in accordance with the present invention and negative controls (cf. Forde et al. 2002). Combining affinity purification and direct full-scan MS in one apparatus, this seemed a viable approach for optimizing pull-down conditions. Preactivated SELDI chips were used to covalently couple Extravidin, a Streptavidin homolog, and were then incubated with a mouse brain nuclei-enriched fraction with or without bioAICD. The washed reaction spots were resolubilized and crystallized in matrix solution, using sinapinic acid (SPA), followed by LDI-TOF. Besides the bait bioAICD peak, some minor differences in the peptide mass range were visible, but for larger proteins, the signal was weak. Further, crude, non-purified cytosolic fractions from mouse brain homogenate were concomitantly analyzed by Coomassie-stained SDS-PAGE and SELDI-TOF using nonspecific absorption, showing this technique to be less suited for the comparison of protein levels in the higher molecular weight ranges; see FIG. 3-2 B of EP 06 025 239.2.

The synthetic peptides purchased from Metabion had been designed with an additional twist: it seemed prudent to use as a negative control for the pull-downs a point-mutated version of AICD as a negative control, based on WB experiments with AICD and Mint proteins (Biederer et al. 2002). Protein binding to the YENPTY motif in these synthetic peptides would be directly proven if they did not bind to the mutated YENATA site. However, for cost reasons and due to the technical difficulties associated with the production of long peptides, only the 21 C-terminal aa of AICD; KMQQNGYENPTYKFFEEQMQN (SEQ ID NO: 2) for the AICD(wt) peptide and KMQQNGYENATAKFFEEQMQN (SEQ ID NO: 3) for AICD(mut) were used, both being preceded by a hydrophilic linker moiety and Biotin for immobilization; see Example 2. However, these peptides contain the most important protein interaction region of AICD and with it one of two structural features of AICD—a type I reverse turn at the NPTY sequence (Kroenke et al. 1997).

The binding, washing and elution procedure was optimized for the peptide bait based purification and for specificity test, WB was performed with X11α and mDab antibodies, both known interaction partners of AICD (supra), on AICD(wt) and AICD(mut) affinity purified eluates from differentiated SH-SY5Y neuroblastoma cell homogenates. After washing with different solutions, as indicated, proteins were eluted by applying 1 M GuHCl, pH 8.0; see FIG. 3-3 of EP 06 025 239.2.

Comparison with Immuno-Precipitation

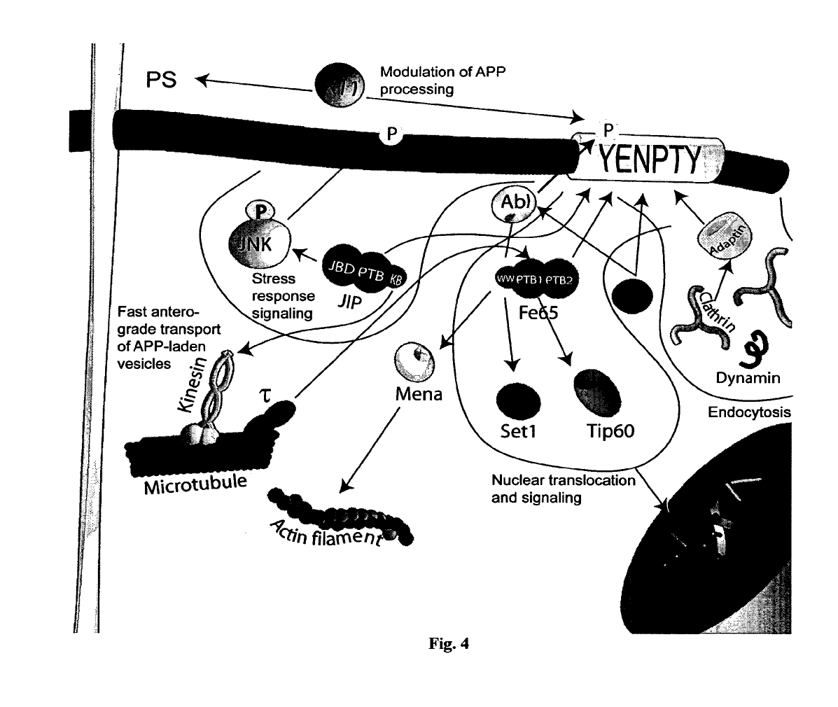

To find out how such bait peptide pull-downs would compare with conventional IPs, using cytosolic fractions from both wt mouse brain homogenates and Hek 293 cell lysates, purifications were performed by normal antibody-mediated IP as described in Example 2. Concerning the choice of antibody, the C-terminal antibody (ab) from Sigma was known to work very well in IP, however, the epitope of this antibody contains the YENPTY region. Therefore, 4G8 monoclonal ab was used for IP of the mouse brain homogenate and 6E10 monoclonal ab for IP of the human cell line derived material. These ab's bind C-terminally proximal to the α-cleavage and β-cleavage sites of APP, respectively. Independently, the same starting material was used to perform bait peptide mediated pull-downs as detailed above and elsewhere (Example 2). Consistently the latter was found to be superior in performance to the IPs, by way of signal to noise ratio, the lack of antibody artifacts and especially total signal strength (see FIG. 3-4 of EP 06 025 239.2). This is probably due to the high molarity of bait peptide, which results in binding of AICD interacting proteins that are in an unbound state at physiological APP levels, and competition with endogenous APP for the remaining factors. Conversely, IPs must typically be performed at lower antibody molarities, and corresponding pull-down yields, for cost reasons. Therefore, the peptide mediated pull-down methodology was used for MS/MS analysis.

The MS/MS data resulting from analysis of SH-SY5Y neuroblastoma cells are presented in Table 1, in a summary of the most important MS/MS data obtained from pull-down experiments in general. There was however a high overlap between the real sample and the negative control, and silver stain gels of AICD(wt)/(mut) based pull-downs showed an excessive number of coinciding bands. Thus, a more specific elution method had to be established to reduce elution of background proteins.

### The PreScission Concept

PreScission protease is a fusion of human rhinovirus 3C and GST and thus binds to glutathione-conjugated sepharose columns. Importantly, it has an 8 aa recognition sequence (L-E-V-L-F-Q¹G-P) (SEQ ID NO: 4) which is extremely rare and it can be safely used with high efficiency and specificity (cf. Kohli and Ostermeier 2003). Therefore, a second set of synthetic AICD peptides was designed that incorporate this sequence at their N-terminus, after the Biotin moiety and the hydrophilic linker region used hitherto. The peptides thus purchased were the following: PrSciAICD(wt)=Biotin—linker—GLEVLFOGPKMQQNGYENPTYKFFEQMQN (SEQ ID NO: 5), PrSciAICD(mut) identical but with YENPTY mutated to YENATA (SEQ ID NO: 6). After affinity binding by the Streptavidin-immobilized peptides, the bound peptides and their interaction partners could thus be cleaved off from the beads, without co-elution of contaminants as depicted in FIG. 6 A. This procedure was tested to verify that background actually is reduced and to monitor cleavage efficiency. Undifferentiated SH-SY5Y lysates undergoing the peptide-mediated purification procedure were analyzed in silver-stained gels and X11α WB. Comparing PreScission-cleaved eluate with the total elution by Lithium Dodecyl Sulfate (LDS) of proteins still sticking to the beads, revealed that background is significantly reduced, at the expense of losing some bound interaction partners due to incomplete cleavage; see FIG. 6 B, lane LDS.

LC-MS/MS Data from Synthetic Bait Peptide Pull-Downs

The general procedure for purification by peptide bait mediated pull-downs has been described supra and in Example 2. By way of measurement, desalted tryptic cleavage-derived peptides were separated on a reverse phase column in an ACN organic solvent gradient prior to injection on LCQ Deca ion traps. A general comment on the presentation of data from respective LCQ measurements from in-solution digests: different analytical software tools were used, but all data presented here was re-analyzed with the newest methods to facilitate comparison throughout, and all proteins shown were identified at the p<0.05 significance level, corresponding to probabilities of correct identification of at least 0.95 (cf. the introduction to expectation maximization model in Example 3). Proteins are listed as a comparison of the actual sample and the negative control. For transparency, a filtering to eliminate proteins that are present in both samples or only in the negative control is not applied. Such a filtering was initially applied directly to Sequest-derived results when PeptideProphet and ProteinProphet were not yet available and was later seen to be problematic, as will be discussed infra, which was one major reason why finally the use of quantitatively comparative methods in the LTQ-FT and MALDI-TOF/TOF measurements according to the present invention was chosen.

With the AICD(wt)/(mut) peptide baits, i.e. without specific elution by cleavage, data from MS/MS analyses of proteins isolated from undifferentiated SH-SY5Y neuroblastoma cell cytosol were obtained; see Table 1. The high overlap of identified proteins between the two samples is testimony to the quality score stringency but also shows that there is a high amount of background in the two samples, with only eight proteins being identified uniquely in the AICD(wt) sample. Also, for several of these, another protein family member was identified in the negative control sample, reducing its relevance. This and the low number of total identifications but high number of identified peptides from highly abundant proteins such as Actin, Myosin and ribosomal proteins are due to overrepresentation of unspecifically bound contaminants, a problem that was addressed by the specific elution by PreScission cleavage as described supra.

TABLE 1
Comparison of proteins identified by AICD(wt)/(mut) pull-down of SH-SY5Y
cell lysate cytosolic fraction

| Swissprot | AICD(wt) | Swissprot | AICD(mut) |
|---|---|---|---|
| | 10 × 40S ribosomal proteins | | 7 × 40S ribosomal proteins |
| | 15 × 60S ribosomal proteins | | 8 × 60S ribosomal proteins |
| P10809 | 60 kDa heat shock protein, mitochondrial | P10809 | 60 kDa heat shock protein, mitochondrial |
| | precursor | | precursor |
| Q5T8M8 | Actin, alpha 1, skeletal muscle | Q5T8M8 | Actin, alpha 1, skeletal muscle |
| P60709 | Actin, cytoplasmic 1 | P60709 | Actin, cytoplasmic 1 |
| P05067 | Amyloid beta A4 protein precursor | P05067 | Amyloid beta A4 protein precursor (only at |
| | | | p = 0.48!) |
| P50454 | Collagen-binding protein 2 precursor | O75531 | Barrier-to-autointegration factor |
| P68104 | Elongation factor 1-alpha 1 | Q6NWZ1 | CKAP4 protein |
| P14625 | Endoplasmin precursor | P50454 | Collagen-binding protein 2 precursor |
| P04406 | Glyceraldehyde-3-phosphate | P68104 | Elongation factor 1-alpha 1 |
| | dehydrogenase, liver | | |
| P04792 | Heat-shock protein beta-1 | P04406 | Glyceraldehyde-3-phosphate |
| | | | dehydrogenase, liver |
| P52272 | Heterogeneous nuclear ribonucleoprotein M | P11142 | Heat shock cognate 71 kDa protein |
| P16403 | Histone H1.2 | P04792 | Heat-shock protein beta-1 |
| Q96BA7 | HNRPU protein, | P52272 | Heterogeneous nuclear ribonucleoprotein M |
| Q9UFZ5 | Hypothetical protein DKFZp434D064 | Q16778 | Histone H2B.q |
| Q9Y427 | Hypothetical protein DKFZp586K2222 | Q96BA7 | HNRPU protein |
| Q9NTK6 | Hypothetical protein DKFZp761K0511 | Q9UFZ5 | Hypothetical protein DKFZp434D064 |
| Q8N390 | Hypothetical protein DKFZp762J227 | Q9NTK6 | Hypothetical protein DKFZp761K0511 |
| P46821 | Microtubule-associated protein 1B | P46821 | Microtubule-associated protein 1B |
| Q9H3F4 | MSTP030 | P35579 | Myosin heavy chain, nonmuscle type A |
| P35579 | Myosin heavy chain, nonmuscle type A | P35580 | Myosin heavy chain, nonmuscle type B |
| P35580 | Myosin heavy chain, nonmuscle type B | P14649 | Myosin light chain 1, slow-twitch muscle |
| | | | A isoform |
| P60660 | Myosin light polypeptide 6 | P60660 | Myosin light polypeptide 6 |
| P19105 | Myosin regulatory light chain 2, | P19105 | Myosin regulatory light chain 2, |
| | nonsarcomeric | | nonsarcomeric |
| P06748 | Nucleophosmin | P67809 | Nuclease sensitive element binding protein 1 |
| Q5T1D1 | OTTHUMP00000017090 | P06748 | Nucleophosmin |
| Q8NC51 | Plasminogen activator inhibitor 1 RNA- | P55209 | Nucleosome assembly protein 1-like 1 |
| | binding protein | | |
| P43490 | Pre-B cell enhancing factor precursor | Q5T1D1 | OTTHUMP00000017090 |
| Q6NZ55 | ribosomal protein L13 | Q6NZ55 | ribosomal protein L13, |
| Q8N6Z7 | ribosomal protein S6 | Q5T8U3 | ribosomal protein L7a |
| Q8WVC2 | RPS21 protein | Q8N6Z7 | ribosomal protein S6 |
| Q15657 | Tropomyosin isoform | Q8WVC2 | RPS21 protein |
| P68363 | Tubulin alpha-ubiquitous chain | P20290 | Transcription factor BTF3 |
| P07437 | Tubulin beta-2 chain | Q5VU66 | Tropomyosin 3 |
| Q9UDW8 | WUGSC:H_DJ0747G18.3 protein | Q15657 | Tropomyosin isoform |
| | | P68363 | Tubulin alpha-ubiquitous chain |
| | | P68371 | Tubulin beta-? chain |
| | | P07437 | Tubulin beta-2 chain |
| | | Q9UDW8 | WUGSC:H_DJ0747G18.3 protein |

For each analysis, four 15 cm plates of confluent SH-SY5Y cells were lysed, bound to AICD(wt)/(mut) resins, eluted in 4 M GuHCl, dialyzed into Trypsin compatible buffer, trypsinized, Zip-Tip desalted and analyzed on a ThermoFinnigan Deca ion trap after reverse phase separation. Two separate runs were performed for each sample and the results combined. The CID spectra were searched against a human Swissprot/Genbank combined database using the Transproteomic Pipeline. Bait peptide, although strongly bound through Streptavidin/Biotin interaction, was detected in both samples and is given underlined. Proteins identified in both samples are given italic. Proteins that are unique to a sample are given without special indication.

If sufficient sample is available, one method to raise the obtainable number of identifications from a sample is to prefractionate the eluted proteins or peptides prior to the RP separation based on hydrophobicity. Therefore, the same purification was performed with the exception of using 4 different GuHCl elution concentrations: 0.25 M, 0.5 M, 1 M, 4 M to yield 4 different fractions for analysis. This analysis required 4 times as much machine time, but as expected, gave far better identification of coverage of the samples; see “supplementary data”, infra. However, the issue of large amounts of background proteins remained.

Therefore, samples from identical starting material were analyzed but with the PreScission protease-based specific elution as described supra designed to reduce background; see also Example 2. This experiment yielded the first putatively physiologically interesting IDs, with Clathrin components exclusively identified in the PrSciAICD(wt) sample. Also, the background-reducing effects of the specific elution step were clearly observed, with only 6.25% of all proteins stemming from Ribosomes, while over 50% of proteins from the experiment depicted in (Table 1) belong to this category. Experimental data from this experiment is shown in Table 14; see “supplementary data”, infra, as data from a similar, more elaborate experiment is detailed directly below.

Instead of relying exclusively on a purely biochemical purification and fractionation, it was also looked at a specific, functionally relevant compartment of AICD (supra): analysis of synaptosomes also seemed a promising experiment from which to gain a set of interesting candidates. Thus synaptosome purifications from wt mice were performed (see Example 2) and the obtained proteins were purified using the previously described standardized PrSciAICD(wt)/(mut) pull-down. This double elimination of background proteins through preparation of synaptosomes and use of the specific PreScission elution brought a clear amelioration, with less analytic capacity wasted on identification of ribosomal or Cytoskeletal proteins. Additionally, Guanine nucleotide-binding protein G(0), a known interaction partner of AICD (supra), was clearly present in the (wt) sample, and with hindsight, the presence of SNAP25 exclusively in the (wt) sample confirms its enrichment in purified samples from the transgenic APP-TAP-AICD mouse as will be described infra.

TABLE 2
Synaptosome preparation in combination with PreScission cleavage yields
increased specificity MS-data and identifies two proteins as unique AICD interactors that
are confirmed in MS-measurements of the transgenic mice of the present invention

| Swissprot | PrSciAICD(wt) | Swissprot | PrSciAICD(mut) |
|---|---|---|---|
| P63260 | Actin, cytoplasmic 2 | Q91XV3 | 22 kDa neuronal tissue-enriched acidic |
| | | | protein |
| Q6NY00 | Aldoa protein, | Q99KI0 | Aconitate hydratase, mitochondrial |
| | | | precursor |
| P12023 | Amyloid beta A4 protein precursor | P68033 | Actin, alpha cardiac |
| Q03265 | ATP synthase alpha chain, | Q6NY00 | Aldoa protein, |
| | mitochondrial precursor | | |
| P56480 | ATP synthase beta chain, mitochondrial | O55042 | Alpha-synuclein |
| | precursor | | |
| Q9DCX2 | ATP synthase D chain, mitochondrial | P12023 | Amyloid beta A4 protein precursor |
| Q9DB20 | ATP synthase oligomycin sensitivity | Q03265 | ATP synthase alpha chain, |
| | conferral protein, mitochondrial | | mitochondrial precursor |
| | precursor | | |
| Q9JKC6 | BM88 antigen | Q9CQQ7 | ATP synthase B chain, mitochondrial |
| | | | precursor |
| Q04447 | Creatine kinase, B chain | P56480 | ATP synthase beta chain, mitochondrial |
| | | | precursor |
| P30275 | Creatine kinase, ubiquitous | Q9DCX2 | ATP synthase D chain, mitochondrial |
| | mitochondrial precursor | | |
| P56391 | Cytochrome c oxidase polypeptide VIb | Q6PIE5 | Atp1a2 protein, |
| Q6P5D0 | Dpysl2 protein, | Q8VCE0 | Atp1a3 protein, |
| Q91VC6 | Glutamine synthetase | Q8CHX2 | ATPase, H+ transporting, V1 subunit A, |
| | | | isoform 1 |
| P18872 | Guanine nucleotide-binding protein G | Q04447 | Creatine kinase, B chain |
| | (o), alpha subunit 1 | | |
| P08249 | Malate dehydrogenase, mitochondrial | P12787 | Cytochrome c oxidase polypeptide Va, |
| | precursor | | mitochondrial precursor |
| Q80U89 | Clathrin heavy chain | Q9D0M3 | Cytochrome c1, heme protein, |
| | | | mitochondrial precursor |
| P04370 | Myelin basic protein | Q6P5D0 | Dpysl2 protein, |
| Q8VEM8 | Phosphate carrier protein, | P43006 | Excitatory amino acid transporter 2 |
| | mitochondrial precursor | | |
| Q9DBJ1 | Phosphoglycerate mutase 1 | P17183 | Gamma enolase |
| P63011 | Ras-related protein Rab-3A | Q91VC6 | Glutamine synthetase |
| Q5U410 | Similar to glyceraldehyde-3-phosphate | P16858 | Glyceraldehyde-3-phosphate |
| | dehydrogenase | | dehydrogenase |
| Q8VCE0 | Sodium\potassium-transporting ATPase | Q6NZD0 | Hspa8 protein |
| | alpha-3 chain | | |
| P14094 | Sodium\potassium-transporting ATPase | P02535 | Keratin, type I cytoskeletal 10 |
| | beta-1 chain | | |
| O88935 | Synapsin-1 | Q64291 | Keratin, type I cytoskeletal 12 |
| P60879 | Synaptosomal-associated protein 25 | P08249 | Malate dehydrogenase, mitochondrial |
| | | | precursor |
| P01831 | Thy-1 membrane glycoprotein precursor | Q80Y13 | Mdh1 protein |
| Q9Z1R9 | Trypsinogen 16 | Q80U89 | Clathrin heavy chain |
| Q5XJF8 | Tubulin, alpha 1 | Q91VD9 | NADH-ubiquinone oxidoreductase 75 kDa |
| | | | subunit, mitochondrial precursor |
| Q7TMM9 | Tubulin, beta 2 | P35802 | Neuronal membrane glycoprotein M6-a |
| Q80Y54 | Tubulin, beta 4 | O55125 | NipSnap1 protein |
| Q9DB77 | Ubiquinol-cytochrome-c reductase | Q8VEM8 | Phosphate carrier protein, |
| | complex core protein 2, mitochondrial | | mitochondrial precursor |
| | precursor | | |
| Q8CHR4 | Vesicle associated membrane protein 2 | P09411 | Phosphoglycerate kinase 1 |
| | | Q61644 | Protein kinase C and casein kinase |
| | | | substrate in neurons protein 1 |
| | | P52480 | Pyruvate kinase, isozyme M2 |
| | | P63011 | Ras-related protein Rab-3A |
| | | O88492 | S3-12 |
| | | P07724 | Serum albumin precursor |
| | | Q8VDN2 | Sodium\potassium-transporting ATPase |
| | | | alpha-1 chain precursor |
| | | P14094 | Sodium\potassium-transporting ATPase |
| | | | beta-1 chain |
| | | P09671 | Superoxide dismutase [Mn], |
| | | | mitochondrial precursor |
| | | O88935 | Synapsin-1 |
| | | P01831 | Thy-1 membrane glycoprotein precursor |
| | | Q9Z1R9 | Trypsinogen 16 |
| | | P05213 | Tubulin alpha-2 chain |
| | | Q9ERD7 | Tubulin beta-3 |
| | | Q7TQD2 | Tubulin polymerization-promoting |
| | | | protein |
| | | Q7TMM9 | Tubulin, beta 2 |
| | | Q80Y54 | Tubulin, beta 4 |
| | | O35619 | Vesicle associated membrane protein 2 |

Wild type mouse brains were homogenized and synaptosomes prepared as described in Example 2. The samples were then purified according to the PreScission protocol and analyzed on an LCQ-Deca as for the previously shown samples. Again, bait peptide was clearly identified in spite of strong binding through Biotin/Streptavidin interaction and is given underlined. Proteins found in both samples at the p < 0.05 significance level according to ProteinProphet are given italic. Again, proteins identified uniquely in one sample are presented without specific indication. Physiologically relevant proteins that are unique in the positive sample are given in bold and were also found in analyses of the transgenic mice of the present invention (Table 5)

At the time of initial analysis, the ProteinProphet statistical analysis software was not yet available, and Guanine nucleotide-binding protein G (0) could not be conclusively assigned to the positive sample. As will be described infra, the constant fine-tuning of the algorithms and scoring mechanisms in new software versions resulted in a slight shift in probability assignment, from 0.94 to 0.97. The data at the time still pointed in the direction of low coverage of identification of the entire sample, and one- and two-dimensional gel electrophoresis was used to further fractionate the PrSciAICD(wt)/(mut) purified proteins prior to trypsinization. The cytosolic protein fraction from one half of a wildtype mouse brain was used for each pull-down experiment and separated either by one-dimensional or two-dimensional gel electrophoresis prior to gel-excision, trypsinization and LC-MS/MS.

However, data quality from these experiments was low. The initial analysis of spot 2 from 2DGE included information on isoelectric point (pI) and molecular weight (MW) as additional filtering criteria and led to the cloning of SHEP1, an SH2-domaining protein which could thus theoretically bind to AICD, for IP validation, as depicted in FIG. 3-6 of EP 06 025 239.2. This co-IP of SHEP for APP was negative, and was probably a result of weighting biochemical (pI and MW) information more strongly than MS quality information. However, an important result from the 2DGE experiment was the visualization of the high complexity of the sample as judged by MS-compatible silver staining of the two-dimensional gel and the small observable differences between the two sample preparations. The use of 2DGE as a general technique did not seem advisable based on several recommendations and due to the rapid growth and success of LC-MS/MS technology. The latter has several important advantages over the former: while 2DGE still gives the best sample separation to date, in-gel Trypsin digestion is much inferior to in-solution digestions in terms of yield and, in consequence, sensitivity and is far more prone to artifacts from sample handling. However, it did show that for the analysis of whole-brain homogenate, several modifications to the sample preparation and analysis setup were required to eliminate background proteins sufficiently for LC-MS/MS analysis.

Accompanying all these alterations in protocol in pursuit of an optimal sample preparation, technical details such as reverse phase elution gradient modifications during LC-MS/MS were also changed, but with negligible effect.

Global representations of quality data from some of the LC-MS/MS measurements performed during experiments in accordance with the present invention revealed that there was a clear difference between LC-MS/MS and gel-derived data; see FIG. 3-7 of EP 06 025 239.2.

### Fulspec

In the following, a summary is provided of the findings on an alternative sampling algorithm for data dependent tandem mass spectrometry (Kohli et al. 2005).

### The Undersampling Issue

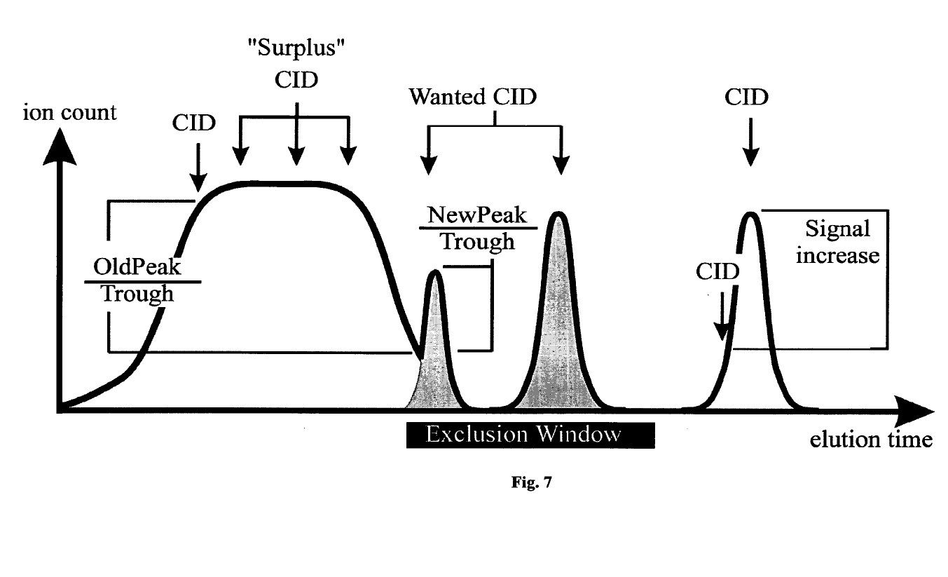

The general technical principles behind liquid chromatography tandem mass spectrometry have been described supra. ThermoFinnigan's LCQ Deca, still the workhorse ion trap apparatus for many a proteomics laboratory, is capable of performing around one full m/z-range scan (MS-scan) or m/z-scan of a fragmented precursor ion (MS²-scan) per second. Typical user settings define one MS-scan followed by two to four MS²-scans as the default analytical work cycle during the chromatography run, resulting in somewhere around 5000 MS²-scans for an entire peptide elution gradient. With proteins typically yielding around 50 tryptic peptides, complex samples can never be exhaustively analyzed within the time frame of a chromatography run, a problem which is called “undersampling”. Even well-resolved chromatography runs show tailing or broad peaks that would waste analytical capacity through repeated CID analysis if the control software were to choose precursor ions for fragmentation and identification based exclusively on intensity. Therefore, current dynamic exclusion mechanisms exclude m/z-values from fragmentation if precursors with this m/z-value have been used for CID already a threshold number of times inside a specific time window. The duration of exclusion from further analysis is the so-called “exclusion window”. Why this current model may miss interesting peaks is explained in FIG. 7 and Table 3.

As any alterations to the control algorithms of MS equipment can only be made by the manufacturer, previously published LC-MS/MS datasets were used to test-run the Fulspec algorithm according to the present invention and described infra. The data are derived from flow-through fractions 35 and 36 of a T cell lipid raft dataset and the cytosolic fraction of liver cells treated with interferon, designated raftflow experiment 35/36 and IFN, respectively (Von Haller et al. 2003; Yan et al. 2004).

A crude global analysis of this data shows how relevant the problem of choice of precursor ions is.

TABLE 3
Reasoning for exploring alternatives to dynamic exclusion

| | Number of | Total number of IDs | |
|---|---|---|---|
| Raft | originally identified | with a PeptideProphet | Oversampled |
| experiment | unique peptides | value >0.5 | peptides |
| 35 | 98 | 463 | 365 (79%) |
| 36 | 119 | 283 | 164 (58%) |

Filtering out peptide sequences that are repeatedly identified shows that many are identified several times (“oversampled peptides”) and thus their corresponding CIDs are of reduced interest and waste analytical capacity. This is in part due to repeated analysis of abundant peptides eluting throughout the LC-MS/MS experiment because of the limited rigid exclusion window used in current machine control software. PeptideProphet is a Bayesian statistics based algorithm (Keller et al. 2002) that allows scoring of peptide identifications (IDs) with absolute probability values, as it has been trained on datasets obtained from protein digests of known compositions.

### The Fulspec Algorithm

Current dynamic exclusion algorithms do not take into account chromatographic information, entailing several problems; see FIG. 7. Contemplating three different possibilities in terms of peptide elution, one can see why this is necessary: for relatively narrow peaks, exclusion is undesirable, as new peptides might elute only fractions of a minute later. For a peak of intermediate length, one high-quality CID spectrum is sufficient, and repeated analyses are wasted MS capacity. Finally, abundant contaminants such as polymers may be present at high intensities throughout the measurement and even the longest limited-duration exclusion window would be insufficient. Fulspec addresses all these issues by adhering to the following set of rules (parameters defined in FIG. 7):
The initial choice of precursor ion depends entirely on total ion count (intensity).
The same peak can henceforth no longer be chosen for CID unless:
   The signal increases by the “signal increase” factor (beginning peak elution).
   The signal first decreases by the “OldPeak/Trough” factor and then increases above the trough value by the “NewPeak/Trough” factor (old peptide replaced by a new chromatographic peptide peak).

Finally, maximum peak intensity can be set for CID consideration, the reasoning for which will be discussed infra.

These rules may seem to assume excessively simple chromatographic principles, i.e. without peak resolution or other parameters being taken into account, but it was also attempted to implement a more elaborate calculus-based rule set relying on moving averages and second derivatives of intensities, but typical LC-MS/MS data were found to be too noisy and compressed for such an analysis.

Details of parameter choice or technical issues such as speed of calculation, (Kohli et al. 2005), and the results from a comparison of different exclusion algorithms with Fulspec, applied to the raftflow datasets, may be taken from FIG. 3-9 of EP 06 025 239.2. Thus, Fulspec chooses several peptides in the highlighted area that are excluded from analysis by conventional algorithms. These analyses are retrospective, as only equipment manufacturers are capable of prospective implementations.

### Signal Intensity and CID Quality

For both models, the implicit assumption up to this point was that the quality of IDs from CID, i.e. the quality of the MS² spectra is linearly dependent on the intensity, i.e. the abundance of the corresponding precursor ion. While this seems plausible based on common sense perception of the way S/N-ratios change in relation to readout intensity, it was decided to challenge this assumption. PeptideProphet absolute probability values from a total of 4869 peptides from the raftflow datasets were correlated with the averaged intensity of the preceding and succeeding MS-scans at the respective m/z-value. Interestingly, it could be observed a) a slightly negative correlation of −0.0575 for raftflow 35 and −0.0299 for raftflow 36 and b) a clear indication that while higher intensity at the lower end of the dynamic range of the instrument yields better IDs, at the upper end, the opposite occurs, with the best peptide identifications derived from precursor ions present at intermediate levels as depicted in FIG. 3-10, A and B of EP 06 025 239.2. This surprising result was validated with the experimentally entirely independent IFN dataset, where the correlation was −0.0244.

For the algorithm used in accordance with the present invention, this resulted in the application of an upper signal threshold limit, which was derived empirically from a frequency distribution analysis of peptide IDs classified as high or low quality, which showed that at the specific threshold chosen, analysis of the raftflow experiments would have resulted in eliminating 16.5% of low quality CIDs, while only losing 0.43% of higher quality CIDs, thus freeing up valuable analytical capacity as depicted in FIG. 3-10, C and D of EP 06 025 239.2. Finally, it was found that the average Fulspec CID intensities, even prior to application of threshold settings, are closer to the averaged intensity of high-quality IDs from the raftflow datasets, compared to the benchmark algorithm.

However, since due to implementation, validation and certification issues at ThermoFinnigan, the ion trap manufacturer was unable to adopt Fulspec, the second aspect of the proteomics approach of the present invention to finding AICD interaction partners had to be re-addressed, namely sample preparation, which led to the focussing on an entirely different purification system, as described in the following.

### The Tandem Affinity Purification Approach (TAP)

As indicated from the above, a new methodology for sample preparation was essential. It should have the potential to yield highly pure protein preparations and still be scalable. One such system is the tandem affinity purification (TAP) system, a generic double affinity protein purification method with a total of four specific binding and elution steps (Rigaut et al. 1999). It entails tagging the protein of interest with a tag consisting of protein A, which binds the target protein to IgG beads in a first step, a TEV protease cleavage site, which allows selectively cleaving the bound protein complex from the first matrix, and a Calmodulin binding peptide, which allows binding to Calmodulin-coupled beads in a second purification step, from which the purified protein complex can be eluted by applying a Ca²⁺ chelator reagent such as EGTA. A commercially available version of this system (Stratagene, #240101) was employed, with the first purification step exchanged with binding of Streptavidin binding peptide (SBP) to Streptavidin coupled sepharose matrix, followed by specific elution by competition with Biotin. With a femtomolar dissociation constant, the Biotin/Streptavidin interaction is the strongest non-covalent interaction known in biology.

### TAP in Cell Culture

One strategy to employ TAP was to transfect human cell-lines producing TAP-tagged AICD that would enable measurements in different cell types, with realization possible in an intermediate timeframe. Two constructs were employed that would allow both pull-down experiments of AICD as well as a negative control, using the empty tag vector for the latter. AICD was cloned into the N-terminal TAP vector under control of the strong eukaryotic CMV promoter (TAP-AICD).

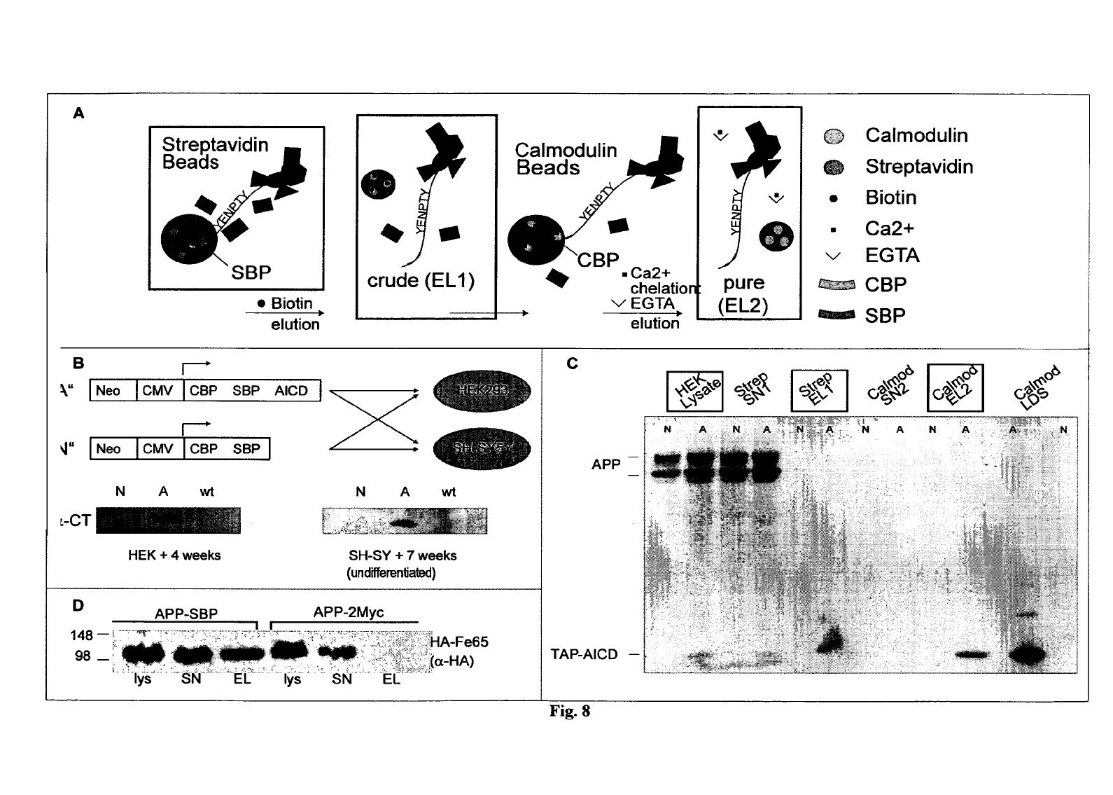

As depicted in FIG. 8, C, only a small fraction of the entire bound protein was eluted from the Calmodulin column with EGTA alone, while the majority was eluted only on addition of gel loading buffer. Therefore, the second affinity purification step required some optimization—100 mM EGTA were found to result in far better yields of TAP-AICD in EL2 than when using the recommended 3 mM EGTA; see Example 2.

### LC-MS/MS Data

Purifications were performed with Hek 293 and SH-SY5Y cells, however only marginally useful data were obtained due to low quality of identifications. This was certainly an issue of final sample amount due to the double purification, as judged from silver stain gels and one reason why the transgenic mouse of the present invention and described in detail in the following was generated and with which data were obtained that were far superior to the data from stably TAP-AICD transfected Hek 293 and SHY-SY5Y cells.

### The Transgenic TAP Mouse

A transgenic mouse was created containing TAP-tagged APP expressed in both neurons and astrocytes that would allow purification of APP and AICD-bound proteins from a physiologically relevant environment. The reasons for this were several-fold; producing full-length APP under the control of the Prion promoter allowed expression of APP under physiological conditions and in brain tissue, which is of more interest than renal or even neuroblastoma cells (HEK293 and SH-SY5Y, respectively). Also, the protein production could theoretically be scaled by breeding, albeit our goal naturally was to obtain as much data as possible from as few mice as necessary.

Due to limited resources, it was not possible to generate mice with a variety of different versions of TAP-tagged AICD or APP, but instead to produce a TAP-tagged version of full-length human APP, due to two reasons:
a) The identities of any bound proteins would allow classifying them as intra- or extracellular proteins and would thus allow the distinction of whether it was a protein that bound to the cytosolic or the extracellular domain of APP, respectively.
b) The expression of full-length APP would ensure physiologically correct protein sorting, trafficking and processing and might thus be less artifact-prone than producing AICD alone.

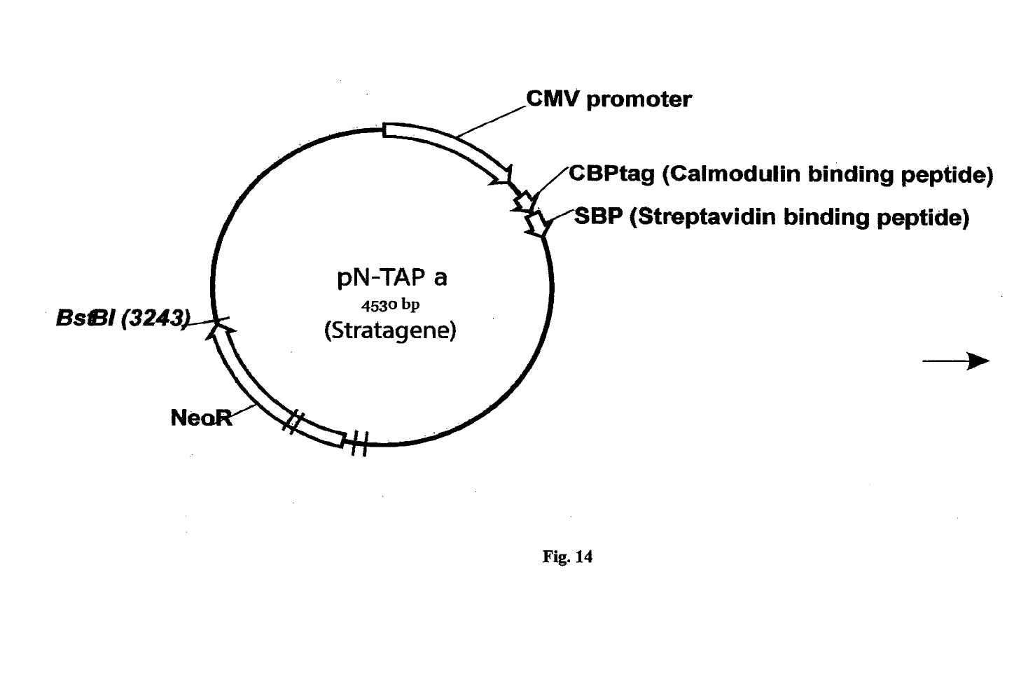

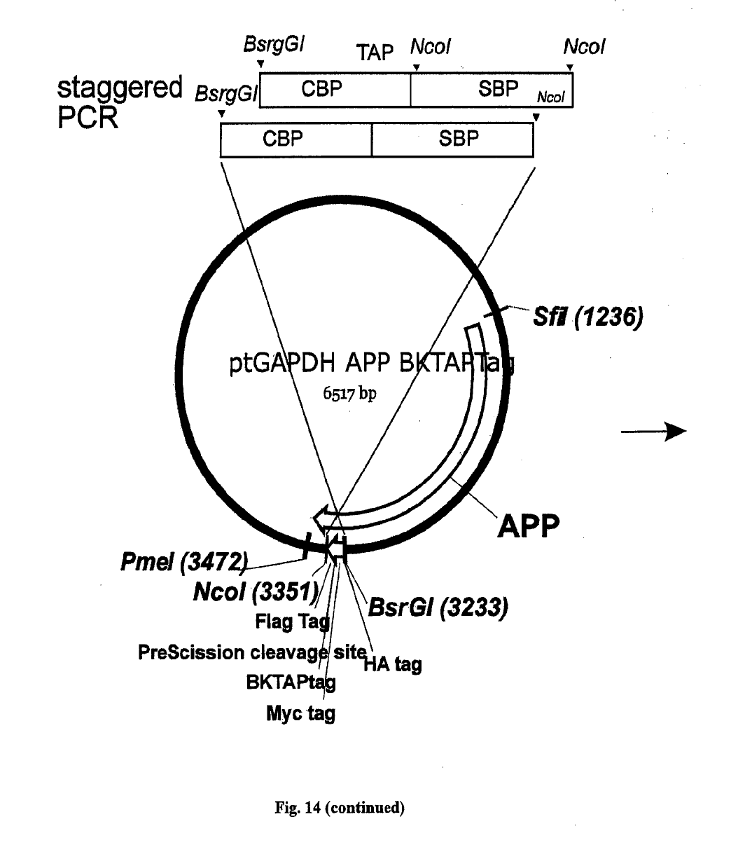

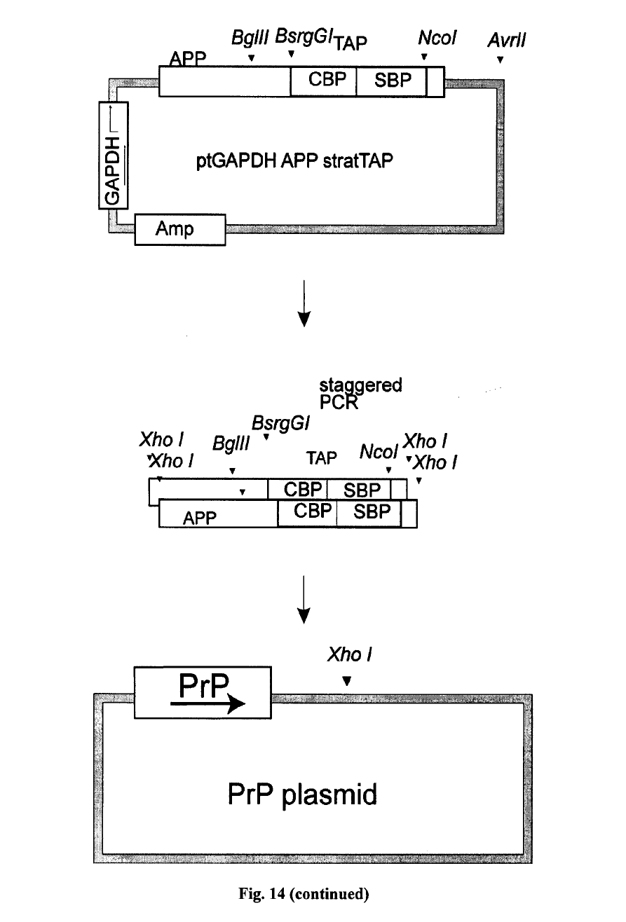

As most proteins that bind to AICD bind to the YENPTY region located at the extreme C-terminus of APP, the TAP tag was entered right after the triple-Lysine membrane insertion stop signal but still very close to the beginning of the AICD sequence, so as not to disrupt binding of interactors. Cloning of our APP-TAP construct is described in Example 7 with an alignment of the C-termini of wt APP and APP-TAP-AICD, and details are schematically depicted in FIG. 14.

The protein purification in Hek 293 cells was tested with transient transfection of the construct driven by a GAPDH promoter. It had to be made sure that protein sorting and trafficking was similar to that of normal human APP by analyzing the subcellular localization of APP-TAP-AICD in Hek 293 cells by fluorescence microscopy, since the purification also worked as for TAP-AICD, confidence was provided that the TAP epitopes were freely accessible to binding in spite of close membrane proximity.

Thus, transgenic mice were generated expressing the fully sequenced APP-TAP-AICD construct. Therefore, three founder lines were generated, with very similar levels of protein, which was produced at similar levels as endogenous APP, as determined by WB comparing 22C11 antibody staining, which detects both APP of mouse and human origin, to 6E10 antibody staining, which detects exclusively human APP, as it binds at the N-terminus of the Aβ region. High overexpression is unwanted, as this leads to excessive co-purification of chaperones during TAP experiments.

Processing of mouse samples required several optimizations compared to purification of cell-culture derived samples, with the second affinity purification working only at higher Ca²⁺ levels for binding and EGTA levels for elution, or with self-coupled Calmodulin beads with high amounts of Calmodulin.

Silver stain gels of purifications from several mice did not show clearly distinct bands between the transgenic mouse and non-transgenic littermate control, with low total protein yields, and data quality obtained from LC-MS/MS analyses of excised gel regions was low, rendering clear identification of proteins unique to the transgenic sample difficult.

It was finally decided to look at eluates from the first affinity purification step alone, as the Streptavidin binding procedure is very reliable and robust, and the samples were found to be more complex, as expected, but to show stronger staining and individual bands stemming from proteins that are clearly more abundant in the APP-TAP-AICD sample compared to the negative control, which was an important prerequisite for LC-MS/MS analysis. Account was taken of the higher sample complexity by, apart from analyzing the samples individually (infra), performing iTRAQ labeling (infra), which in terms of quantitation eliminates the undersampling issue (supra) for samples that are to be compared directly.

LTQ Data from the APP-TAP-AICD Mouse

ThermoFinnigan's LTQ is an advanced linear ion trap: its large trap volume reduces space charge effects that limit trapping capacity in 3D ion traps such as the LCQ we used for the measurements performed in accordance with the present invention and commonly yields approximately twice as many IDs as the latter (Riter et al. 2006). Combination of this apparatus with an FT mass analyzer additionally yields highest accuracy for the mass of the precursor ion. Finally, another technique has been used that allows semiquantitative comparison of protein levels in different samples using LTQ-FT equipment (“Semiquantitative method”), some aspects of sample preparation of which and the actual data obtained are presented, as well as a comparison of this data with the complementary MALDI-TOF/TOF measurements in Table 7.

Proteins from the first elution step (EL1) from mice 72 (−) and 75 (+) (m72/m75) were reduced, and their Cysteine groups methylated prior to tryptic digestion and final C₁₈-based purification. Sample aliquots and digests were analyzed by silver staining densitometry to roughly measure the relative sample amounts. A conventional LTQ run was performed for fine-tuning of the total peptide amounts based on the average base ion peak intensities and total ion counts but also yielded several protein identifications used to cross-check protein IDs from the “Semiquantitative method” mentioned above. Using this technique the 10% of proteins that were most clearly enriched in the m75 sample were extracted. Their identifications are presented in the following list (Table 4). In terms of quality, it is currently not possible to analyze this dataset directly with ProteinProphet for technical reasons, but all peptides denoted as “peptides matched” in the following list have PeptideProphet derived probabilities of >0.9. Also, these enriched proteins were annotated with ProteinProphet derived probabilities from proteins identified in the LTQ measurement of the sample from mouse 75. Thus, it is clear that most proteins were positively identified, as there is a very good overlap of the two measurements.

TABLE 4
Proteins found to be more abundant in transgenic mouse 75 after Streptavidin-
Biotin purification and analysis by LTQ-FT and the “Semiquantitative method”

| | | | | LTQ |
|---|---|---|---|---|
| Semiquantitative method (LTQ-FT) | | | | ProtProph |
| | | probability | | p-value |
| | | that this | | from |
| | | protein is | Matched | initial |
| NCBI-ID | Protein name | enriched | peptides | LTQ-run |
| A4_MOUSE | Amyloid beta A4 protein precursor (APP) | 1.00 | 7 | 1 |
| EAA1_MOUSE | Excitatory amino acid transporter 1 (Sodium- | 1.00 | 2 | 0.95 |
| | dependent glutamate/aspartate transporter) (High- | | | |
| | affinity neuronal glutamate transporter) (Glial high | | | |
| | affinity glutamate transporter) (GLAST) | | | |
| ENOG_MOUSE | Gamma-enolase (EC 4.2.1.11) (2-phospho-D- | 1.00 | 2 | 1 |
| | glycerate hydro-lyase) (Neural enolase) (Neuron- | | | |
| | specific enolase) (NSE) (Enolase 2) | | | |
| Q80TR2_MOUSE | MKIAA0820 protein (Fragment) | 1.00 | 2 | 0.91 |
| 1433F_MOUSE | 14-3-3 protein eta | 1.00 | 6 | 1 |
| 1433T_MOUSE | 14-3-3 protein theta (14-3-3 protein tau) | 1.00 | 2 | 0.9 |
| 1433E_MOUSE | 14-3-3 protein epsilon (14-3-3E) | 1.00 | 4 | 1 |
| PROF1_MOUSE | Profilin-1 (Profilia I) | 1.00 | 2 | 0.97 |
| SODC_MOUSE | Superoxide dismutase [Cu—Zn] | 1.00 | 2 | 1 |
| PEBP_MOUSE | Phosphatidylethanolamine-binding protein | 1.00 | 6 | 0.98 |
| | (PEBP) (HCNPpp) [Contains: Hippocampal | | | |
| | cholinergic neurostimulating peptide (HCNP)] | | | |
| PRDX5_MOUSE | Peroxiredoxin-5, mitochondrial precursor (Prx-V) | 1.00 | 2 | 1 |
| | (Peroxisomal antioxidant enzyme) (PLP) | | | |
| | (Thioredoxin reductase) (Thioredoxin peroxidase | | | |
| | PMP20) (Antioxidant enzyme B166) (AOEB166) | | | |
| | (Liver tissue 2D-page spot 2D-0014IV) | | | |
| ACON_MOUSE | Aconitate hydratase, mitochondrial precursor (Citrate | 1.00 | 8 | 1 |
| | hydro-lyase) (Aconitase) | | | |
| ALDOA_MOUSE | Fructose-bisphosphate aldolase A (Muscle-type | 1.00 | 11 | 1 |
| | aldolase) (Aldolase 1) | | | |
| EF1A2_MOUSE | Elongation factor 1-alpha 2 (EF-1-alpha-2) | 1.00 | 4 | n.a. |
| | (Elongation factor 1 A-2) (eEF1A-2) (Statin S1) | | | |
| NP_034610 | similar to heat shock protein 1, alpha isoform 1 | 1.00 | 4 | 1 |
| MYLK2_MOUSE | Myosin light chain kinase 2, skeletal/cardiac muscle | 1.00 | 2 | n.a. |
| | (MLCK2) (Fragment) | | | |
| SH3G1_MOUSE | SH3-containing GRB2-like protein 1 (SH3 domain | 1.00 | 2 | n.a. |
| | protein 2B) (SH3p8) | | | |
| PEP19_MOUSE | Brain-specific polypeptide PEP-19 (Brain-specific | 1.00 | 2 | n.a. |
| | antigen PCP-4) (Purkinje cell protein 4) | | | |
| 1433B_MOUSE | 14-3-3 protein beta/alpha (Protein kinase C | 1.00 | 2 | 0 |
| | inhibitor protein 1) (KCIP-1) | | | |
| ATP5I_MOUSE | ATP synthase e chain, mitochondrial | 0.88 | 2 | 0.82 |
| AT1A2_MOUSE | Sodium/potassium-transporting ATPase alpha-2 chain | 0.88 | 11 | 1 |
| | precursor (Sodium pump 2) (Na+/K+ ATPase 2) | | | |
| | (Alpha(+)) | | | |
| Q9CY54_MOUSE | 13 days embryo liver cDNA, RIKEN full-length | 0.88 | 6 | 1 |
| | enriched library, clone: 2500004H04 product: | | | |
| | Hemoglobin, beta adult major chain, full insert | | | |
| | sequence | | | |
| 2AAA_MOUSE | Serine/threonine-protein phosphatase 2A 65 kDa | 0.88 | 2 | 0.96 |
| | regulatory subunit A alpha isoform (PP2A, subunit A, | | | |
| | PR65-alpha isoform) (PP2A, subunit A, R1-alpha | | | |
| | isoform) | | | |
| Q6NZF5_MOUSE | Lrrk1 protein (Fragment) | 0.88 | 1 | n.a. |
| SNP25_MOUSE | Synaptosomal-associated protein 25 (SNAP-25) | 0.88 | 2 | 1 |
| | (Synaptosomal-associated 25 kDa protein) (Super | | | |
| | protein) (SUP) | | | |
| STXB1_MOUSE | Syntaxin-binding protein 1 (Unc-18 homolog) | 0.88 | 16 | 1 |
| | (Unc-18A) (Unc-18-1) | | | |
| CN37_MOUSE | 2′,3′-cyclic-nucleotide 3′-phosphodiesterase (CNPase) | 0.87 | 5 | 1 |
| Q9CRC1_MOUSE | Adult male testis cDNA, RIKEN full-length enriched | 0.87 | 2 | n.a. |
| | library, clone: 4933425L11 product: fructose- | | | |
| | bisphosphate aldolase A homolog | | | |
| GPM6B_MOUSE | Neuronal membrane glycoprotein M6-b (M6b) | 0.87 | 2 | 0.74 |
| PGAM1_MOUSE | phosphoglycerate mutase 1 | 0.86 | 2 | n.a. |
| NSF_MOUSE | N-ethylmaleimide sensitive fusion protein (NSF) | 0.86 | 8 | 1 |
| MDHM_MOUSE | Malate dehydrogenase, mitochondrial precursor | 0.86 | 17 | 1 |
| AT2B2_MOUSE | Plasma membrane calcium-transporting ATPase 2 | 0.86 | 2 | 1 |
| | (PMCA2) (Plasma membrane calcium pump isoform | | | |
| | 2) (Plasma membrane calcium ATPase isoform 2) | | | |
| RAB1A_MOUSE | Ras-related protein Rab-1A (YPT1-related | 0.85 | 2 | n.a. |
| | protein) | | | |
| AP180_MOUSE | Clathrin coat assembly protein AP180 (Clathrin | 0.85 | 2 | n.a. |
| | coat-associated protein AP180) (91 kDa | | | |
| | synaptosomal-associated protein) | | | |
| 1433Z_MOUSE | 14-3-3 protein zeta/delta (Protein kinase C | 0.83 | 4 | n.a. |
| | inhibitor protein 1) | | | |
| 1433G_MOUSE | 14-3-3 protein gamma | 0.83 | 6 | 0.99 |
| DYN1_MOUSE | Dynamin-1 | 0.82 | 2 | 1 |

Amyloid Precursor Protein, using this method, was at the top of list (cf. Table 8), given underlined. Other already known interaction partners of AICD or putatively biologically relevant proteins are given in bold. Proteins that are reasonably sure to be enriched were included in this list (p < 0.15). As the scoring function assigning probability of enrichment with this method is still being fine-tuned, five further proteins are included that have slightly lower probability of being enriched (0.82-0.85) but are functionally relevant or were also found in the iTRAQ sample. Even with the assigned probabilities, 4 of these 5 proteins are enriched.

iTRAQ/MALDI Data from the APP-TAP-AICD Mouse

With the “Semiquantitative method”, this data were complemented with iTRAQ labeling-derived quantitative data. One half of the sample from mice 72 and 75 that was used for LTQ analysis was retained and the negative control sample was labeled with the 114.1 Da reporter reagent and the mouse 75(+) sample with the 116.1 Da iTRAQ reagent before mixing the individual samples and spotting the combined sample through reverse phase chromatography fractionation onto MALDI plates.

Using a Visual Basic script written for the purpose, iTRAQ 116/114 ratios were calculated for all proteins that had been identified at the p<0.05 significance level according to Mascot; see Example 3. The identified proteins were grouped into two groups according to whether their abundance in the m75 sample, i.e. their iTRAQ 116/114 ratio was higher or lower than the average over all 116/114 ratios. The former group is depicted in Table 5. Importantly, APP itself was clearly identified with seven peptides as an enriched protein (iTRAQ ratio=4.65), as expected based on qualitative Western Blot analysis. Further, several known interaction partners of AICD are present on this list (cf. supra). Taken together, these validating facts and the quantitative nature of the dataset suggest that proteins on this list are clearly enriched during purification of APP and its C-terminal proteolytic fragments, whether through direct or indirect interactions.

All proteins in this list (Table 5) were additionally screened for protein identifications that were extremely likely to be correct according to following highly stringent criteria: proteins that had been identified by at least two peptides that were each uniquely assignable to this protein and of which at least one had a Mascot score ≧25 are marked with bold font protein scores. Proteins known to interact with AICD or of putative biological and physiological relevance are given in bold. One of these candidates, Dynamin, was of high interest to us in the context of differential nuclear signaling by α-CTFs and β-CTFs, and its influence thereupon was analyzed in cell culture experiments. Further below, the data will additionally be discussed in the context of the LTQ-derived dataset and brought into a cell biological perspective.

TABLE 5
Proteins that were found to be present at higher-than-average abundance in
Streptavidin purified samples from APP-TAP-AICD expressing
transgenic mouse 75 vs. control mouse 72

| | | Mascot | Peptide | iTRAQ |
|---|---|---|---|---|
| NCBI ID | (Swissprot ID) Protein description (synononyms) | Score | matches | ratio |
| MDHC_MOUSE | (P14152) Malate dehydrogenase; cytoplasmic | 80 | 2 | 5.96 |
| HBA_MOUSE | (P01942) Hemoglobin alpha chain | 125 | 3 | 5.96 |
| KCRB_MOUSE | (Q04447) Creatine kinase; B chain | 166 | 12 | 5.32 |
| SYN1_MOUSE | (O88935) Synapsin-1 | 110 | 5 | 5.23 |
| ATPB_MOUSE | (P56480) ATP synthase beta chain; mitochondrial precursor | 265 | 14 | 5.02 |
| PEBP_MOUSE | (P70296) Phosphatidylethanolamine-binding protein | 51 | 2 | 4.87 |
| | (PEBP) | | | |
| KPYM_MOUSE | (P52480) Pyruvate kinase; isozyme M2 | 94 | 11 | 4.71 |
| MDHM_MOUSE | (P08249) Malate dehydrogenase; mitochondrial precursor | 76 | 6 | 4.71 |
| AT1A1_MOUSE | (Q8VDN2) Sodium/potassium-transporting ATPase alpha-1 | 289 | 13 | 4.71 |
| | chain precursor (Sodium pump 1) | | | |
| UCHL1_MOUSE | (Q9R0P9) Ubiquitin carboxyl-terminal hydrolase isozyme L1 | 40 | 2 | 4.68 |
| | (UCH-L1) | | | |
| A4_MOUSE | (P12023) Amyloid beta A4 protein precursor (APP) | 131 | 10 | 4.65 |
| TPIS_MOUSE | (P17751) Triosephosphate isomerase (TIM) (Triose-phosphate | 151 | 5 | 4.61 |
| | isomerase) | | | |
| MPCP_MOUSE | (Q8VEM8) Phosphate carrier protein; mitochondrial precursor | 42 | 3 | 4.61 |
| | (PTP) | | | |
| ALDOA_MOUSE | (P05064) Fructose-bisphosphate aldolase A (Muscle-type | 154 | 6 | 4.61 |
| | aldolase) (Aldolase 1) | | | |
| STX1C_MOUSE | (P61264) Syntaxin-1B2 | 103 | 3 | 4.57 |
| DYN1_MOUSE | (P39053) Dynamin-1 | 65 | 11 | 4.57 |
| G3P_MOUSE | (P16858) Glyceraldehyde-3-phosphate dehydrogenase | 281 | 11 | 4.54 |
| | (GAPDH) | | | |
| ACON_MOUSE | (Q99KI0) Aconitate hydratase; mitochondrial precursor | 86 | 9 | 4.53 |
| | (Citrate hydro-lyase) | | | |
| 1433Z_MOUSE | (P63101) 14-3-3 protein zeta/delta (Protein kinase C | 260 | 13 | 4.51 |
| | inhibitor protein-1) (KCIP-1) (SEZ-2) | | | |
| G6PI_MOUSE | (P06745) Glucose-6-phosphate isomerase (GPI) | 53 | 6 | 4.48 |
| | (Phosphoglucose isomerase) (PGI) | | | |
| EAA2_MOUSE | (P43006) Excitatory amino acid transporter 2 (Sodium- | 121 | 7 | 4.44 |
| | dependent glutamate/aspartate transporter 2) | | | |
| SNP25_MOUSE | (P60879) Synaptosomal-associated protein 25 (SNAP-25) | 68 | 5 | 4.41 |
| | (Synaptosomal-associated 25 kDa protein) | | | |
| STXB1_MOUSE | (O08599) Syntaxin binding protein 1 (Unc-18 homolog) | 82 | 9 | 4.36 |
| | (Unc-18A) (Unc-18-1) | | | |
| 1433G_MOUSE | (P61982) 14-3-3 protein gamma | 190 | 12 | 4.28 |
| HS90A_MOUSE | (P07901) Heat shock protein HSP 90-alpha (HSP 86) (Tumor | 97 | 6 | 4.22 |
| | specific transplantation 86 kDa antigen) | | | |
| AT1B1_MOUSE | (P14094) Sodium/potassium-transporting ATPase beta-1 chain | 115 | 10 | 4.14 |
| AT1A3_MOUSE | (Q6PIC6) Sodium/potassium-transporting ATPase alpha-3 | 464 | 25 | 4.14 |
| | chain (Sodium pump 3) | | | |
| ENOG_MOUSE | (P17183) Gamma enolase (2-phospho-D-glycerate hydrolyase) | 95 | 5 | 4.08 |
| | (Neural enolase) | | | |
| VAM2_MOUSE | (P63044) Vesicle-associated membrane protein 2 (VAMP- | 74 | 3 | 4.04 |
| | 2) (Synaptobrevin 2) | | | |
| AATC_MOUSE | (P05201) Aspartate aminotransferase; cytoplasmic | 59 | 5 | 4.04 |
| | (Transaminase A) | | | |
| NSF_MOUSE | (P46460) Vesicle-fusing ATPase (EC 3.6.4.6) (Vesicular- | 39 | 9 | 4.00 |
| | fusion protein NSF) (N-ethylmaleimide sensitive fusion | | | |
| | protein) | | | |
| LDHB_MOUSE | (P16125) L-lactate dehydrogenase B chain (LDH-B) (LDH | 38 | 5 | 3.99 |
| | heart subunit) | | | |
| ATPG_MOUSE | (Q91VR2) ATP synthase gamma chain; mitochondrial | 47 | 4 | 3.98 |
| | precursor | | | |
| CLCA_MOUSE | (O08585) Clathrin light chain A (Lca) | 48 | 3 | 3.98 |
| 1433E_MOUSE | (P62259) 14-3-3 protein epsilon (14-3-3E) | 82 | 8 | 3.94 |
| DPYL2_MOUSE | (O08553) Dihydropyrimidinase related protein-2 (DRP-2) | 212 | 10 | 3.92 |
| | (ULIP 2 protein) | | | |
| ENOA_MOUSE | (P17182) Alpha enolase (2-phospho-D-glycerate hydro-lyase) | 150 | 10 | 3.90 |
| | (Non-neural enolase) (NNE) | | | |
| SYT1_MOUSE | (P46096) Synaptotagmin-1 (Synaptotagmin I) (SytI) (p65) | 61 | 3 | 3.82 |
| VATA1_MOUSE | (P50516) Vacuolar ATP synthase catalytic subunit A; | 32 | 5 | 3.81 |
| | ubiquitous isoform | | | |
| ALDOC_MOUSE | (P05063) Fructose-bisphosphate aldolase C (Brain-type | 88 | 6 | 3.77 |
| | aldolase) (Aldolase 3) | | | |
| GLNA_MOUSE | (P15105) Glutamine synthetase (Glutamate-ammonia ligase) | 105 | 9 | 3.76 |
| SH3G1_MOUSE | (Q62419) SH3-containing GRB2-like protein 1 (SH3 | 40 | 2 | 3.64 |
| | domain protein 2B) (SH3p8) | | | |
| AATM_MOUSE | (P05202) Aspartate aminotransferase; mitochondrial precursor | 51 | 3 | 3.51 |
| | (Transaminase A) | | | |
| PPIA_MOUSE | (P17742) Peptidyl-prolyl cis-trans isomerase A (PPIase) | 147 | 6 | 3.46 |
| | (Rotamase) (Cyclophilin A) | | | |
| ATPA_MOUSE | (Q03265) ATP synthase alpha chain; mitochondrial precursor | 125 | 14 | 3.39 |
| GNAO1_MOUSE | (P18872) Guanine nucleotide-binding protein G(o); alpha | 34 | 6 | 3.38 |
| | subunit 1 | | | |
| TBA2_MOUSE | (P05213) Tubulin alpha-2 chain (Alpha-tubulin 2) | 399 | 14 | 3.32 |
| HSP7C_MOUSE | (P63017) Heat shock cognate 71 kDa protein (Heat shock 70 kDa | 198 | 11 | 3.27 |
| | protein 8) | | | |
| TBB4_MOUSE | (Q9D6F9) Tubulin beta-4 chain | 451 | 16 | 3.22 |

Brain homogenates from mouse 75 and mouse 72 were purified through preclearing, binding to Streptavidin sepharose and competitive elution by Biotin as described in Example 2, with WB of aliquots. These samples were processed according to the iTRAQ workflow described in Example 3, and spotted through reverse phase chromatography fractionation for MALDI-TOF/TOF analysis on an ABI 4800. Searches were performed against the mouse protein database; see Example 3. Shown are all proteins that have high quality scores and are enriched above average in the m75 sample. Purified bait protein is given underlined. Other already known interaction partners of AICD or putatively biologically relevant proteins are given in bold. As full normalization over the entire measurement was not possible through the GPS explorer software suite, this conservative estimate had to be taken for determining the enriched vs. the non-enriched proteins. Proteins that were identified by at least two uniquely assigned peptides of which at least one had a Mascot score ≧25 are marked with a bold font protein score. Caution regarding specificity is required for two proteins: SH3-containing GRB2-like protein 1 could also be SH3-containing GRB2-like protein 2, and VAMP2 could also be VAMP3. Ranking is in order of abundance ratio.

Supporting the approach of the present invention and the quality of the data, several of the proteins found here, especially among those given in bold for their putative functional relevance, are published interactors with either clear biochemical evidence for such an interaction (Table 6, group I) or at least MS data that has previously been published (Table 6, group II). Thus, Dynamin I was not a novel interactor of AICD, but apart from proving the interaction itself, so far no additional function data has been published.

TABLE 6
Several of the physiologically interesting proteins found to be enriched
in pull-downs of APP and its C-terminal fragments have previously
been shown to interact with AICD:

| | Reference |
|---|---|
| Group I | |
| Clathrin light chain | (Chen et al. 1990; Lai |
| | et al. 1995) |
| Guanine nucleotide-binding protein G(0) | (Nishimoto et al. 1993) |
| Group II | |
| 14-3-3 gamma and zeta, NEM sensitive fusion | (Cottrell et al. 2005) |
| protein, Syntaxin binding protein 1, Dynamin I | |

Functional and biochemical validations are shown in group I, MS data and pull-downs only pertain to group II.

This still leaves several previously unidentified interactors of APP open to validation in the future. A summary and consolidation of the most interesting leads to pursue in the future, besides Dynamin, as will be discussed infra.

The pUKBK Vector System

Plasmid vectors are an important tool in the analysis of proteins in cell-culture, both biochemically and microscopically. However, for the special requirements of the method of the present invention, the currently available plasmids had several shortcomings:
a) size: most expression plasmids are above 5 kb, without insert. Transfection efficiency is inversely proportional to size and, especially for monitoring the effects of APP processing on AICD signaling, where we transfected up to three different plasmids simultaneously, we needed high transfection efficiencies.
b) selection: typical expression plasmids, especially the smaller ones, do not support stable selection by antibiotics in eukaryotic cells. This was one aspect required for generating the stable APP-Citrine cell line (cf. supra).
c) tagging: rapid swapping of affinity, staining or fluorescence tags on cDNA clones: A modular vector was desired that would allow the insertion of interesting APP interactors or processing modifiers directly into the vector of choice for the experimental issue at hand.

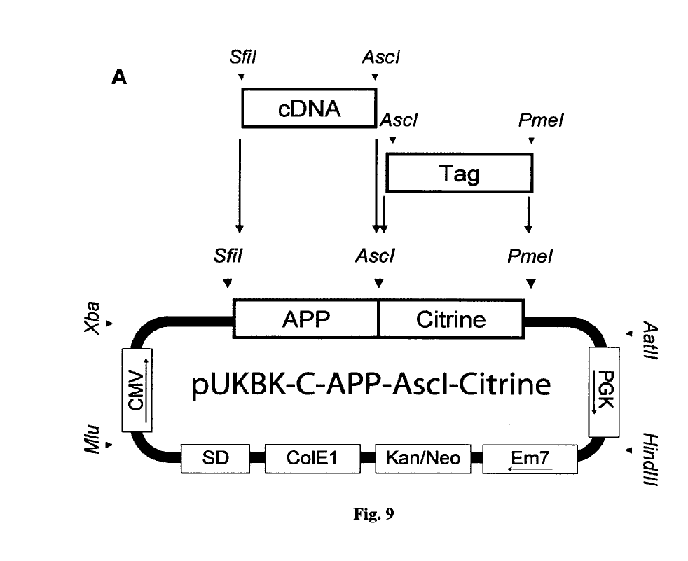

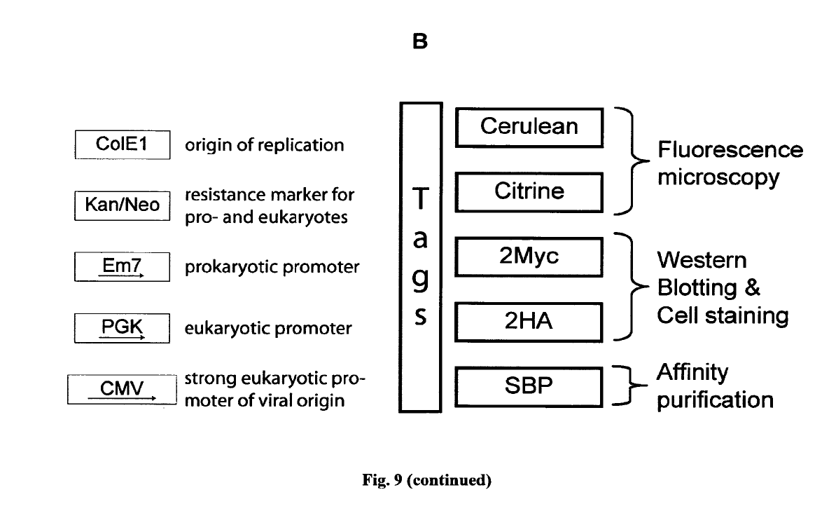

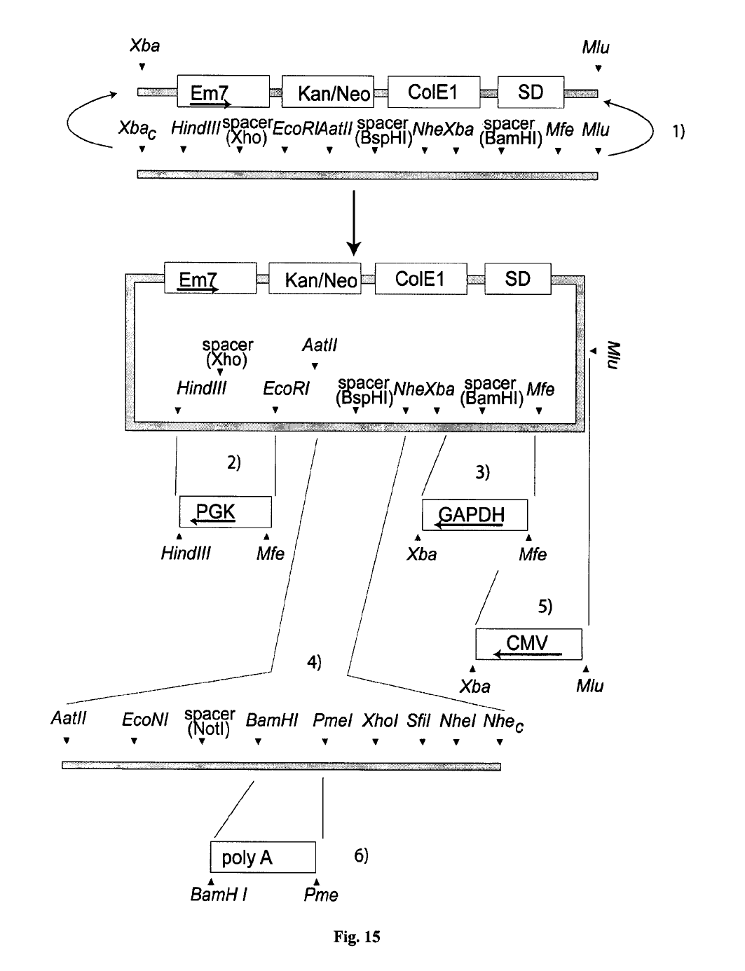

The construction and features of a vector system that tackles these issues and has become the choice tool when performing cell-biological experiments ever since its inception are described in FIG. 15 and FIG. 9, respectively.

### Effects of APP Trafficking/Processing on Nuclear Signaling by AICD

### Overview

Previously published experiments showed that AICD cleaved from APP translocates to the nucleus, forms distinct nuclear complexes with Fe65 and Tip60 and is transcriptionally active (Von Rotz et al. 2004). Further experiments were set up to determine if α- or β-cleavage preceding AICD differentially affects nuclear signaling. Therefore it was looked at different systems mimicking or precluding this choice of RIP pathway, as described in the following.

Follow-up of a mass spectrometry candidate: Dynamin One of the APP-interacting protein candidates that were identified in the proteomics approach (supra) was Dynamin. This protein is central in the GTP-hydrolyzing pinching-off step of vesicles during receptor-mediated endocytosis and thus plays a role in the transfer of APP to endosomes where BACE cleavage would be dominant (supra). Wildtype (wt) Dynamin as well as the K44E Dynamin mutant where the GTP binding consensus sequence is altered (Herskovits et al. 1993), both fused to an HA tag were used for microscopic detection.

First distribution of APP-Citrine in Hek 293 cells was monitored and afterwards the extent to which the Dynamin mutant altered the distribution of APP in these cells.

The two different Dynamin versions were transfected into a clonal APP-Citrine cell line and confocal microscopy was performed after Cy5-staining of the Dynamin-HA tag. More APP was localized to cytoplasmic vesicular structures for wt Dynamin and more homogeneous membrane bound distribution of APP for the dominant negative Dynamin K44E mutant (Dyn-K44E) was observed. These differences are clearly visible inside the mutant experiment when comparing cells with strong transfection levels lying directly next to cells with low transfection levels.

In a second step, it was determined whether this disruption of endocytosis would have an influence on formation of tripartite nuclear spots with Tip60 and Fe65. As described supra, Fe65 shuttles AICD to the nucleus and interacts there with the Histone acetyl-transferase Tip60 to form transcriptionally active nuclear complexes. Von Rotz et al. 2004 also showed γ-secretase inhibition to reduce the formation of these complexes, and according to the present invention microscopical observation of this AICD/Fe65/Tip60 (AFT) spot-formation was used as one of the read-outs in the experiments.

Thus, Tip60-CFP and Fe65 N-terminally tagged with a Myc tag (Myc-Fe65) were cotransfected together with either Dynamin or Dyn-K44E into the APP-Citrine clonal cell line. the number of nuclei showing spherical AFT spots was counted and found to be strongly reduced in those wells where Dyn-K44E was transfected vs. wt Dynamin. With a total of three biological replicates, this result is to be significant at the p<0.05 level using the conservative Mann-Whitney nonparametric test. For the second and third experiment, control experiments (blind experiments) correctly identified the Dyn-K44E transfected wells.

### RT-PCR of AICD Target Genes: A Validation

Apart from using the number of cells that contain AFT spots as readout for AICD signaling to the nucleus, also RT-PCR readout was wanted based on genes that are known to be transcriptionally regulated by AICD. RT-PCR is very difficult to employ when a) changes are around or below 2-fold, especially when considering that b) transfection never affects the entire cell population, which, while sufficient for WB and biochemistry, is insufficient to detect weak transcriptional changes by RT-PCR as these effects can be cancelled or at least averaged out by the untransfected cells. It was known that Kai 1 (Baek et al. 2002) and APP (Von Rotz et al. 2004) were regulated by the ternary AFT complex. In RT-PCR experiments, the upregulation of APP was not strong enough to yield significant differences in the experimental setups used, so some of the genes previously found by Ruth von Rotz to be regulated by AICD expression alone in Hek 293 cells were validated in microarray experiments (ETH Diss. No 15893) to find optimal genes for the RT-PCR readout according to the present invention. Even though Transcription Elongation Factor A showed the strongest response to AICD induction in a clonal Hek 293 cell line, variation was higher than for the second- and third-best genes and we thus decided to use the published AICD target gene Kai1, Prolactin Receptor (Pro1R) and Chr13 Orf18 (C13O18) as readout in the following experiments.

### α- and β-CTF “Precleaved” Constructs

As described supra, APP is cleaved by α-secretase into an 83 aa 9.2 kDa α-C-terminal fragment (CTF) or a 99 aa 11.1 kDa β-CTF, in the case of APP₆₉₅. In extension of the Dynamin experiments performed in accordance with the present invention, one question was whether such precleaved CTFs expressed in Hek 293 cells would show differences in localization of AICD to the nucleus.

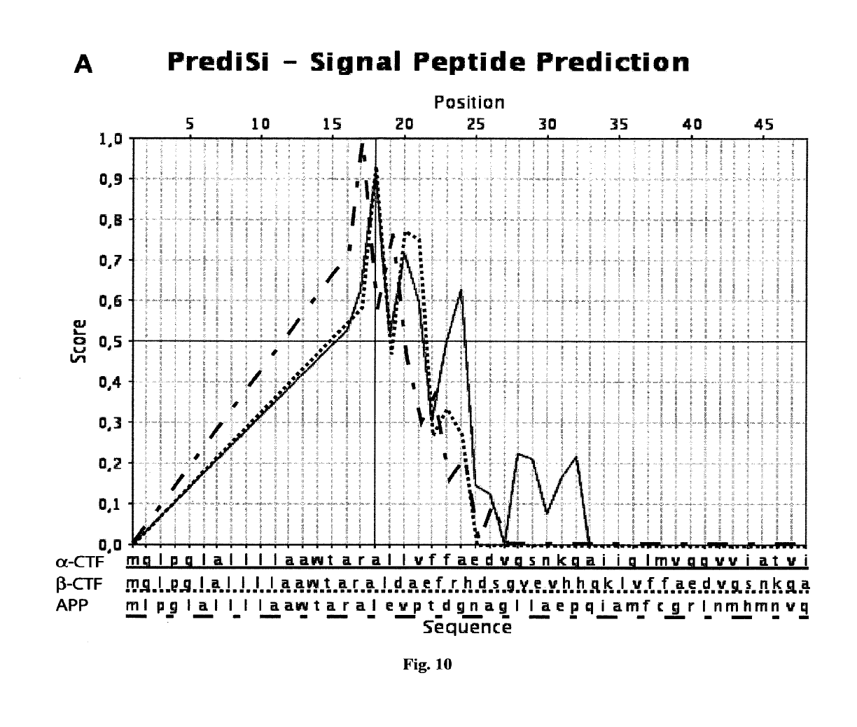

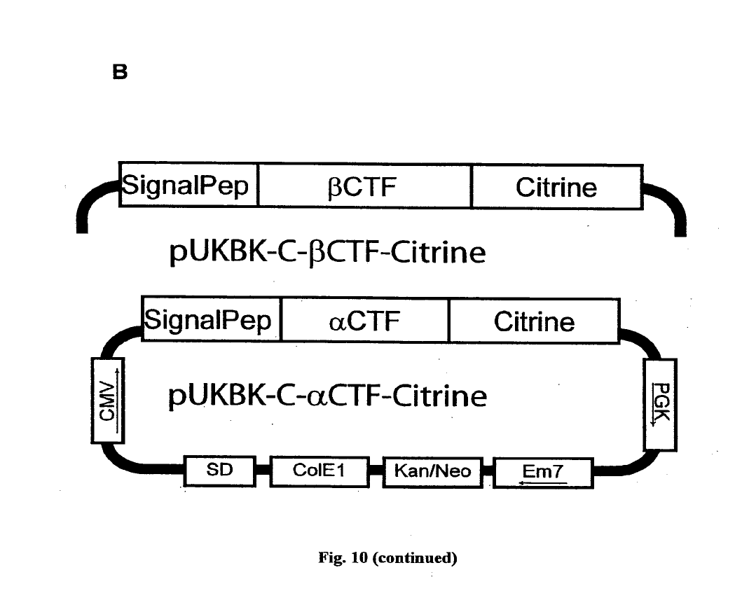

In order to initially obtain as physiological a distribution as possible, essentially including correct membrane insertion, the native APP signal peptide was cloned in front of the α-CTF and β-CTF. These in turn were fused to Citrine at the C-terminus for microscopical visualization purposes; see FIG. 10, right.

The probability of this chimera composed of native signal peptide and APP-CTF being cleaved was assessed using a recently described algorithm (PrediSi (Hiller et al. 2004)), which showed a) the leading signal peptide to presumably still be recognized as such and b) the most favored cleavage position to still be proximal to the beginning of the CTF sequence (FIG. 10, left).

In a first step, the CTF constructs alone were transfected, without any additional influence on subcellular localization and an entirely homogeneous distribution of Citrine fluorescence throughout the cell was observed. Like APP, these constructs first require cleavage of the signal peptide by Signal Peptidase. However, these “precleaved” CTFs never require ectodomain-shedding in the conventional sense of α- or β-secretase cleavage, which is time consuming not only because of the cleavage step alone, but because of the protein trafficking required to make APP accessible to the secretases. PS cleavage was therefore assumed to take place much more rapidly than is the case for wtAPP, whereupon the same experiments were performed with γ-secretase inhibitor L-685,458 aiming at prolonging the half-life of the uncleaved CTFs. Now, accumulation in ER and Golgi was observed, as is visible in the case of APP-Citrine expression. Apart from this stabilization, however, no obvious difference was discerned between the subcellular localization of the two constructs.

Furthermore, the Citrine labeled CTF constructs were transfected into Hek 293 cells together with Fe65 and Tip60 to see whether they would both result in formation of ternary nuclear complexes. Both were capable of producing AFT complexes in the nucleus. However, with quantification of the number of cells with AFT complexes and correcting for transfection efficiency based on total Citrine fluorescence, it was found that there were significantly more cells with tripartite complexes in the β-CTF experiments (n=3).

Also RT-PCR experiments were performed to find whether or not there were observable differences in gene expression. To this end, Hek 293 cells were transfected with either of the two CTF constructs for 24 h prior to harvesting, without Tip60-CFP and HA-Fe65 due to low transfection efficiency when performing triple transfections. Gene expression of Kai 1, C13O18 and ProlR from three independent biological experiments was normalized to GAPDH and Actin expression. The trend corresponded with the microscopy data for two of the three reporter genes but was not significant in Mann-Whitney nonparametric testing at the p<0.05 level.

While this was probably due to the fact that two other interaction partners of AICD that are required for nuclear signaling were only present at endogenous levels, it was nevertheless clear that further validation of a possible difference between α-secretase and β-secretase-derived AICD translocation to the nucleus would require additional experimental setups.

### α- and β-Cleavage Mutants

One further method to differentiate between the amyloidogenic and the non-amyloidogenic pathway is to mutate the secretase-interaction characteristics of APP. Based on two publications analyzing the effect of mutations at and proximal to the α- or β-cleavage site, APP was used containing the H609D/K612E (De Strooper et al. 1993) and M596V (Citron et al. 1995) mutations inhibiting α- and β-cleavage respectively. The respective clones were obtained by site directed mutagenesis (“α-knockdown” (KD) or “β-KD”, respectively) and fused to C-terminal Citrine tags for visualization in fluorescence microscopy.

The relative number of cells in which AFT spots formed were assessed in Hek 293 cells after triple transfection with either of the two cleavage-inhibiting constructs and both HA-Fe65 and Tip60-CFP, and including in the analysis wt APP-producing cells. The APP version containing the β-secretase inhibiting M596V mutation consistently resulted in formation of fewer cells with nuclear spots, while the mutant carrying the α-cleavage inhibiting mutations was on a par with wt APP.

For RT-PCR, the Citrine tag was swapped with an HA tag in place of Citrine using the pUKBK vector system, and Kai 1, C13O18 and ProlR gene expression was compared between α-KD and β-KD APP, using the averaged expression of PGK and GAPDH for normalization. The expression of these reference genes is not significantly changed between the two experimental conditions, while all three AICD target genes showed reduced expression when β-secretase cleavage was inhibited (n=3 and p<0.05). Finally, Western Blot analysis was performed with lysates from APP-HA (β-KD, α-KD and wt) transfected cells that had been additionally treated with γ-secretase inhibitor to allow accumulation of CTFs, showing the effect of the mutations.

### β-Secretase Inhibitor Assays

Several BACE inhibitors have been developed. Such a tri-peptide based inhibitor was purchased with an IC₅₀ of 700 nM that had previously been tested in cell culture (Abbenante et al. 2000). A dilution series of this inhibitor, ranging from 2-fold IC₅₀ down to DMSO vehicle only, was applied to a Hek 293 cell-line constitutively expressing APP-Citrine and again a reduced formation of nuclear AFT spots was found as soon as the non-amyloidogenic pathway was favored. The experiments were performed in triplicate and the number of cells containing AFT spots was significantly different between the vehicle-treated and the 2-fold IC₅₀ treated cells at the p<0.05 level according to Mann-Whitney. The number of cells containing nuclei with ternary AFT spots correlates negatively with the concentration of the inhibitor (correlation=−0.984).

In order to monitor whether the experiments conducted were within concentration range that results in a clear inhibition of the formation of β-CTF by β-secretase, also wt Hek 293 cells were treated with this β-secretase inhibitor using the same dilution series as for the fluorescence microscopy experiments. Additionally, so as to allow detection of CTFs in WB, Hek 293 cells were treated with the γ-secretase inhibitor DAPT and the processing of endogenous APP was analyzed with Western Blot analysis. The clear shift in β-CTF to α-CTF ratio from β-CTF/total CTF=27% for the vehicle-only condition to 9.4% for the 2×IC50 condition, based on densitometry, verified that the dilution series actually does encompass the relevant range.

As already described above, a new transgenic mouse model has been generated expressing TAP-tagged APP at physiological levels. Using mass spectrometry (MS) analysis, several proteins involved in synaptic vesicle endo- and exocytosis to interact with APP have been identified. Further, one of these proteins, Dynamin, was analyzed for its influence on nuclear signaling of APP, leading to the discovery that RIP-mediated APP signaling to the nucleus may depend on whether APP is processed by the amyloidogenic pathway or not, adding yet another twist to its diverse functionality.

### Proteomics Approach to Finding APP Interaction Partners

### Methodological Considerations

Based on previous work of Kohli and Ostermeier 2003, where His-tag purification had been successfully employed, this initially seemed like a feasible way to prepare peptides for pull-down experiments, since besides cost and availability issues, one advantage would have been the possibility of introducing specific mutations modifying the YENPTY site or inserting negatively charged residues mimicking phosphorylation by site directed mutagenesis. The reasoning behind this was the possibility of monitoring phosphorylation dependent binding to AICD (cf. (Ando et al. 2001)). Typically, exogenous proteins driven by the T7 promoter make up nearly 50% of the entire cellular protein (Studier and Moffatt 1986), which clearly was not the case in the experiments performed in connection with the present invention. Also, establishing and optimizing chromatography-based purification instead of batch purifications would have required a concerted and time-consuming effort, negating the previously mentioned benefits.

Thus, as an alternative, chemically synthesized bait peptides were used for the pull-downs performed in accordance with the present invention. SET1, a nucleosome assembly factor that interacts with part of the Fe65 WW-domain, was isolated and identified using a similar peptide-bait based approach as was used for pull-down of AICD—interacting proteins (Telese et al. 2005). The discovering group shows silver stain gels from these pull-downs with only one single protein band, corresponding to SET1. In spite of using several different washing and elution procedures, as well as the specific elution by bait peptide cleavage via PreScission protease, this degree of specificity was never attained. Still, the signal intensities of purified interaction partners of AICD were significantly stronger when using peptides as bait than when using conventional IPs. Also, the specific binding of the AICD interacting proteins mDab and X11α to the nonmutated form of bait peptide was clearly shown, proving the validity of using synthetic bait peptides for pull-downs.

In order to reduce contaminant or “background” proteins, the peptide bait purification according to the present invention was re-designed to allow specific elution by PreScission protease, as described in FIG. 6. In 2DGE experiments with this modified protocol, however, it turned out that even with this method, purification of whole brain homogenates from mice resulted in highly complex samples with only few visible differences between the PrSciAICD(wt) and the PrSciAICD(mut) negative control sample, which led to the concept of tandem affinity purification:

In a comprehensive study on TNF-α signaling (Bouwmeester et al. 2004), Hek 293 cells were not only transfected with 32 tagged bait proteins known to be involved in the signaling pathway, but an additional 50 that were transfected iteratively upon discovery of interactions with previously identified proteins involved in the pathway, in a total of 237 purifications. Since the tandem affinity purification (TAP) technology was shown to be successful (Rigaut et al. 1999), which allows purification of pull-down material to a high degree, a commercially available, slightly modified version thereof was implemented for the purposes of the present invention; see FIG. 8, A.

However, no transient transfection was used to prepare cell-culture material for TAP purifications for MS, as the amount of chaperones and heat shock proteins that are purified under such conditions is far higher than when using stably transfected cell-lines (Brian Raught, ISB, Seattle, personal communication). Again, the pull down of known interaction partners of APP was possible (FIG. 8). In order to enable scalable purification in a more physiologically relevant environment, a transgenic mouse was established producing APP-TAP-AICD under the ubiquitously active Prion promoter, resulting in a unique in vivo application of the TAP-tag approach; see Example 7. Due to the unreliability of the second purification step and the de facto infinite dilutions involved, however, only one single purification step was performed, which yielded large enough eluate protein concentrations to show clear differences between the sample and the negative control. Finally, semi-quantitative MS methods eliminated the undersampling issue discussed in the context of “Fulspec”, below.

### MS Data

Surface enhanced laser desorption/ionization time of flight analysis (SELDI-TOF) had previously been used to detect the presence of a specific DNA interacting protein which was subsequently analyzed by conventional MS analysis from affinity purified protein (Forde et al. 2002). SELDI-TOF was found to be unsuited for the purposes of the present invention for two reasons: a) typical interaction partners of AICD are much larger than the 32 kDa protein, and SELDI was shown to be insensitive in higher MW regions. b) SELDI-TOF does not allow for CID measurement, which is required for obtaining peptide sequence information, and as discussed supra, tandem MS of in-solution digests has become far more important to proteomics than peptide mass fingerprinting from excised gel-derived proteins.

As described supra, several basic concepts of tandem MS were discussed. For the choice of instruments suitable for the purposes of the present invention, several characteristics were important. While ion trap apparatus' such as the LCQ-Deca from ThermoFinnigan have relatively low resolving power and mass accuracy, the very principle of ion traps allows accumulation of precursor ions before fragmentation and results in good sensitivity, which has been additionally increased with the advent of linear ion traps (Riter et al. 2006). Currently, for proteomics methods such as those used in the present invention, i.e. when posttranslational modifications are not an imminent issue, two types of apparatus are probably most suited: Fourier-Transform Ion Cyclotron Resonance (FT-ICR) apparatus' coupled to linear ion traps, such as ThermoFinnigan's FT-LTQ, and MALDI-TOF/TOF machines, such as the Applied Biosystems 4800 (Domon and Aebersold 2006). The former combines the rapid and sensitive tandem MS capabilities of modern linear ion traps and the exceptional accuracy of precursor ion mass from the FT instrument (ppm and sub-ppm range), raising the probability of correct protein identification (IDs). The choice between these two instruments also depends on the method of quantification, if such is used—for iTRAQ labeling, which results in the dissociation of very small (114 Da-117 Da) reporter ions during MS/MS, the extended mass range of MALDI-TOF/TOF instrumentation is required. Thus, LCQ-Deca equipment was used for early experiments, LTQ-(FT) and for iTRAQ measurements the AB4800.

For the analysis of MS-data from the peptide pull-down experiments (supra), several obstacles had to be overcome. The data presented in Table 1 and Table 2 are the result of more recent analyses made once probability calculations with PeptideProphet and ProteinProphet were possible. Initial analyses were done entirely with Sequest, which gives a peptide match and score to each collision induced dissociation spectrum (CID). An earlier program not discussed here grouped peptides common to a single protein, but there was no absolute or quantitative measure for accepting a protein ID as correct. As it is not feasible to visually inspect thousands of CID spectra, the dependence on software for data analysis is high. Small changes in the databases or program versions used can make significant differences. One example from the data obtained according to the present invention is the identification of Guanine-nucleotide binding protein G(0), a known interaction partner of AICD: in an older database search of the synaptosome data (Table 2), the protein was identified with a probability of 0.94, which narrowly missed the quality criteria. In the newest data analysis, the value assigned by ProteinProphet was 0.97, which was in accordance with the p<0.05 a significance criterion. Later experiments with samples from the transgenic mouse of the present invention confirmed this result (Table 5). The combination of database searching (Yates et al. 1995) and scoring of peptide (Keller et al. 2002) and protein (Nesvizhskii et al. 2003) IDs by absolute probability in the “Transproteome Analysis Pipeline” (TPP, Example 3) carries the possibility of standardized analysis and cross-experiment comparisons. The reason for presenting the iTRAQ data according to the present invention in Mascot-format is that the TPP has not yet been adapted to MALDI-data.

Data analysis software is not the only situation where informatics play a role in MS. Acquisition of MS/MS data, i.e. the choice of which ion precursors to fragment by CID, is an important task performed by any LC-MS/MS system. Especially with complex samples, it is imperative that the control software does not waste analytical capacity repeatedly on precursor ions from which good data has already been obtained. Furthermore, Fulspec (Full-scan based peak exclusion) was developed, an alternative sampling algorithm that takes chromatographic principles into account and attempts not to repeatedly pick ions for CID from the same elution peak, as described supra and in Kohli et al. 2005. Finally, the finding in accordance with the present invention that precursor-intensity does not necessarily correlate with CID quality had implications for the experimental procedures of the present invention, as described in the context of semiquantitative comparisons below. Combining better sampling of underrepresented peak areas with quality-correlated intensity thresholds, Fulspec should make the most of LC-MS/MS sample analysis, once hardware-implemented.

In view of the strong differences in WB signal strength of proteins binding to AICD(wt) and AICD(mut), initially a filter was programmed to eliminate from the data of the present invention all proteins that were not identified exclusively in the AICD(wt) or PrSciAICD(wt) sample. However, this led to miss potentially interesting candidates for AICD-interaction partners such as SNAP-25, which was later identified as a clearly enriched protein in both LTQ-FT and iTRAQ measurements on MALDI-TOF/TOF equipment (Table 4 and Table 5). In the synaptosome preparation (Table 2), peptides from SNAP-25 were identified five times in the PrSciAICD(wt) sample and only once in the PrSciAICD(mut) sample, but still the filter eliminated the protein, as it was “present” in both samples. In ideal affinity purifications, there would be no interacting protein in the negative sample and the filter according to the present invention would be entirely effective. However, as the Fulspec data show, high-quality CID spectra can be obtained over a large intensity range, which is why even proteins that bind spuriously to the negative control can give a clear identification. A further reason why such a stringent filter may have been incompatible with the negative control was that not all proteins binding to the YENPTY region of AICD may show equally reduced binding to YENATA as do X11α and mDab, and surrounding aa could also play a role in binding.

Being aware of this possibility, empty vector was used producing the TAP cassette as a negative control, while simultaneously the raise of the standard of purification was sought when designing the TAP experiments of the present invention. Similarly, in the transgenic mouse of the present invention expressing APP-TAP-AICD (see Example 2), PCR-negative littermates were used as negative controls.

Best MS results were obtained with iTRAQ methodology from the transgenic mice, in line with the findings from Fulspec suggesting that changes in precursor ion quantity would hardly reflect on protein IDs (discussed supra). Also, the low percentage of overlap between two different measurements of similar samples hints at the problems of comparing IDs between sample runs. 41% (13 of 32) of proteins identified from the AICD(wt) sample shown in Table 8, ribosomal proteins excluded, were again identified in an identically purified sample that had been fractionally eluted and for which the results are shown in the “supplementary data” section (Table 13), even though the total number of proteins identified in this second sample was nearly 3-fold (94 vs. 32). There are several ways to ameliorate this, which were sequentially pursued: further fractionation of samples, purifying samples more strongly or labeling samples and subsequently mixing them for simultaneous analysis. Fractionations were performed using gel electrophoresis, but, however, protein analysis from the weakly stained small spots could not be analyzed at a high level of confidence. Further purification was also undertaken, using specific elution with PreScission protease (FIG. 6) or by resorting to TAP purification (for a discussion of these topics, see supra). Analyses of samples from the latter, using the transgenic mouse of the present invention and using only the first, highly robust Streptavidin/Biotin purification step, yielded the best results. The presence of APP in both “enrichment lists” obtained using the transgenic mouse of the present invention is a prerequisite for defining a successful purification. Between the iTRAQ and the LTQ-FT dataset thus derived, there is a good overlap of physiologically interesting proteins that are found to be present at higher levels in the APP-TAP-AICD purified samples:

TABLE 7
Physiologically relevant proteins that were identified by both LTQ-FT and
iTRAQ MALDI-TOF/TOF semi-quantitative measurements to be
enriched in purifications from the transgenic mouse

| Swissprot ID | Name | ITRAQ | LTQ |
|---|---|---|---|
| 1433E_MOUSE | 14-3-3 protein epsilon (14-3-3E) | [symbol] | [symbol] |
| 1433G_MOUSE | 14-3-3 protein gamma | [symbol] | [symbol] |
| 1433Z_MOUSE | 14-3-3 protein zeta/delta (Protein kinase C inhibitor protein | [symbol] | [symbol] |
| | 1) (KCIP-1) (SEZ-2) | | |
| A4_MOUSE | Amyloid beta A4 protein precursor (APP) | [symbol] | [symbol] |
| [symbol] | [symbol] | [symbol] | [symbol] |
| [symbol] | [symbol] [symbol] | [symbol] | [symbol] |
| PEBP_MOUSE | Phosphatidylethanolamine-binding protein (HCNPpp) | [symbol] | [symbol] |
| [symbol] | [symbol] [symbol] | [symbol] | [symbol] |
| | [symbol] | | |
| [symbol] | [symbol] [symbol] | [symbol] | [symbol] |
| [symbol] | [symbol] | [symbol] | [symbol] |
| [symbol] | [symbol] [symbol] | [symbol] | [symbol] |
| | [symbol] | | |
| [symbol] | [symbol] | [symbol] | [symbol] |
| [symbol] | [symbol] | [symbol] | [symbol] |
| [symbol] | [symbol] [symbol] | [symbol] | [symbol] |
| | [symbol] | | |
| [symbol] | [symbol] | [symbol] | [symbol] |
| [symbol] | [symbol] [symbol] | [symbol] | [symbol] |
| 1433B,F,T_MOUSE | 14-3-3 proteins beta, eta, theta | [symbol] | [symbol] |

14-3-3 and associated proteins (italic and underlined) are adaptor proteins involved in several signaling pathways, APP (underlined) was the bait protein, and proteins involved in vesicle endo- and exocytosis are given italic and in bold (all proteins listed in alphabetical order). Method(s) detecting enrichment: as indicated on the right. Proteins listed below the bold line were identified as enriched in the APP-TAP-AICD sample based on a single method.

Three proteins that fit into the category of proteins involved in endo- and exocytosis were found solely in the iTRAQ sample: Synapsin-1 and Synaptotagmin-1 and Vesicle Associated Membrane Protein 2. One component that is essential for the formation of coated pits during endocytosis was calculated as enriched in each measurement: Clathrin light chain A in the iTRAQ sample and Clathrin coat assembly protein Aβ180 in the LTQ-FT measurement. On the other hand, the 14-3-3 family was more strongly covered in the LTQ sample, with 14-3-3 β, η and θ (beta, eta and theta) additionally present.

Recently, an AD proteomics study was published that reported on proteins binding to APP in post-mortem brains of healthy control subjects and of AD patients (Cottrell et al. 2005). However, there were no clear differences of APP-binding proteins between the two sample groups, but several novel APP-interacting proteins were found. As summarized in Table 6, their data and that of others validate several of the findings of the present invention.

Some statistics from the data of the present invention help determine the extent to which the categories that were used to describe the enriched proteins are correctly identified. a) In the iTRAQ measurement, 10 enriched proteins are involved in vesicle endo- or exocytosis (20.4% of all enriched proteins), while only one such protein is present at below-average levels (only 2.2%). Also, the latter single protein is ranked 7th of 45 and is thus only weakly underrepresented in the purifications of the transgenic mouse sample. b) 52% of proteins that fit into the categories defined in Table 7 were found to be enriched in purified APP-TAP-AICD samples in both the iTRAQ and the LTQ measurements, compared to an overlap of only 17% for all other enriched proteins. This lends some credibility to the assertion that proteins involved in endo- and exocytosis, as well as the 14-3-3 proteins lie at the core of the data of the present invention. This suggests APP to play an important role in vesicle trafficking in the brain, in concordance with localization of APP to neuronal growth cones and synapses (Ferreira et al. 1993; Sabo et al. 2003). Three modes of synaptic vesicle cycling are currently discerned: sorted by decreasing speed of release and increasing capacity, these are termed “kiss-and-stay”, “kiss-and-run” and endosomal recycling (Sudhof 2004). In the third mode, which is especially required during higher stimulation frequencies (Richards et al. 2000), Clathrin-mediated endocytosis occurs. Via adaptor proteins such as AP180, Clathrin forms a coat around clusters of receptors and other proteins containing endocytosis sequences such as the YENPTY-region of APP (Chen et al. 1990; Guenette et al. 1999). Dynamin is essential for pinching off vesicles during endocytosis, dependent on its GTP-binding domain (Herskovits et al. 1993). SH3p8, perhaps less well known, has been found in a Y2H screen to interact with Dynamin and expression has been shown to be high in nerve terminals (Ringstad et al. 1997). Also, the finding that Dynamin, Clathrin and SH3p8 are associated with APP in pull-down experiments measured by MS correlate with previous findings that APP is endocytosed from presynaptic axon termini (Marquez-Sterling et al. 1997). The findings of the present invention additionally are congruent with their data that shows APP from synaptosomes not to co-purify with Synaptophysin insofar as Synaptophysin was not detected in the experiments of the present invention either. However, the enrichment of a group of factors directly involved in exocytosis extends their proposal that APP is just coincidentally endocytosed with synaptic vesicle proteins but afterwards is sorted off to the soma. Among these factors identified are Syntaxin-1B2, Synaptosome associated protein/soluble NSF-attachment protein (SNAP-25) and the Synaptobrevin VAMP2 (this latter only identified by iTRAQ, Table 3-5), which together make up the core SNARE complex (Chen et al. 2002). N-ethyl-maleimide sensitive factor (NSF), a further essential component for SNARE-mediated membrane fusion (Sollner et al. 1993) and the Syntaxin interacting protein Unc 18 homolog (Hata et al. 1993) were identified in both MS analyses. Finally, Synaptotagmin was identified in the iTRAQ measurement, which brings in the Ca²⁺-dependent component to this fusion apparatus, with its 5 binding sites for Ca²⁺, which can explain the high amount of cooperativity during Ca²⁺ influx and neurotransmitter release (Sudhof 2004). This association of many proteins involved in synaptic vesicle cycling with APP (Table 7) provides further evidence for APP involvement in synaptic function. Therefore, the mouse model of the present invention, coupled with synaptosome preparations, is beneficial for elucidating exactly at which stages APP is involved in the excitatory process. Several of the proteins discussed above were also identified in the bait peptide pull-downs from a mouse brain synaptosome preparation (Table 2; Synapsin 1, VAMP 2, Clathrin heavy chain) but no quantitative information is available on these proteins. However, a further retrospective confirmation of the data described above is the fact that in these synaptosome-derived samples, SNAP 25, a central component of the SNARE complex, was found exclusively in the PreSciAICD(wt) sample.

A further question is, how the group of 14-3-3 proteins fits in, and whether there may be a connection to the “synaptic vesicle”-group. In general, the 14-3-3 protein family mediates three main effects by interacting with other proteins: regulating enzyme activity, regulating subcellular localization and regulation of protein-protein interactions (van Hemert et al. 2001). Recently, it was found that 14-3-3 γ dimers can interact with both AICD and Fe65, facilitating nuclear signaling in a T668 (APP₆₉₅-numbering) phosphorylation dependent fashion (Sumioka et al. 2005). This proves the 14-3-3 proteins' second functional category to pertain to the physiology of APP and is nicely mirrored in the fact that 14-3-3 γ is one of three 14-3-3 proteins that were common to both the LTQ and iTRAQ measurements of the present invention.

There is evidence of 14-3-3 ζ inhibiting UV-induced apoptosis but it is uncertain whether this is through direct physical interaction with JNK (Xing et al. 2000), which has been shown to modulate the apoptotic pathway (Lin and Dibling 2002) and can phosphorylate AICD through interaction with JIP1 (Inomata et al. 2003). 14-3-3 ζ has been shown to interact with an important component of the mitogen activated protein kinase cascade (MAPK) pathway (Koyama et al. 1995). Therefore a further link between the 14-3-3 proteins and the data of the present invention may be that Phosphatidylethanolamine-binding protein is synonymous with Raf1 kinase inhibitor. Finally, it is reasonable to assume that there is a link between the two main groups of APP/AICD-associated proteins. For example, the 14-3-3 protein's ability to modulate protein—protein interactions could be one possibility to act as scaffold proteins between APP and the exocytosis machinery, which would functionally link all proteins in Table 7.

Throughout all the purifications and MS measurements performed in accordance with the present invention, Fe65, Jip1, X11α or mDab were not detected as interactors of APP or AICD. Although it could have been shown that Fe65 can bind to AICD under the buffer conditions employed in the TAP purification process (FIG. 8), Fe65 was only seen in WB of pull-down eluates when it was transiently transfected, i.e. overexpressed. This and the fact that the proteins we found to associate with AICD were not typically detected using Y2H screens shows the complementarity of methods: Y2H technology can inherently detect interactions even of low abundance proteins, but only physical isolation of bait proteins with their associated proteins and MS analysis thereof can identify protein complex components under physiological conditions. Further, it was not enriched specifically for nuclear proteins but a holistic approach was performed to identify interaction partners of APP and AICD: by isolating physiologically expressed APP from brain tissue, APP was pulled down with its natural interaction partners in their normal abundance ratios. Y2H screens detect any possible interaction between two overexpressed proteins, even though one of the partners may be vastly less abundant in vivo.

### APP Processing and Effects on Transcription

### Evidence for β-Secretase Mediated Signaling

The subcellular localization and trafficking of APP is of great importance to its processing. Aβ secretion is strongly reduced in Chinese Hamster Ovary (CHO) cells when endocytosis is disrupted (Koo and Squazzo 1994), even though the secretion of sAPPβ into the medium is not impaired. This suggests that γ-secretase cleavage of CTF-β occurs in endosomes and can be inhibited by blocking endocytosis, as was shown by Carey et al (Carey et al. 2005). However, some data are now going even further, suggesting β-cleavage to occur exclusively after endocytosis. In contrast to β-CTF production, it was reported that production of sAPP-α occurs at the plasma membrane (Sisodia 1992). As γ-secretase is present in the plasma membrane (Tarassishin et al. 2004), besides in intracellular compartments such as ER and Golgi (Annaert et al. 1999), AICD produced from α-CTF can therefore be generated at the plasma membrane, i.e. spatially distinct from AICD derived from β-CTF. This possibly results in different proximity to the nucleus of AICD produced from the two distinct precursors and differing ability to interact with nuclear shuttling proteins. Finally, microscopy analysis of late endosomes detected the presence of APP, suggesting that the main fraction of endocytosed APP is mostly not recycled to the plasma membrane (Ferreira et al. 1993), in line with the assertion that it is cleaved instead and translocates to the nucleus. Thus intracellular trafficking, modulated by several of the proteins identified in the MS analyses according to the present invention (Table 7), have an important effect on the localization of AICD-generation. With translocation of AICD to the nucleus depending on interaction with additional factors such as Fe65 (Kimberly et al. 2001; Von Rotz et al. 2004) and 14-3-3 γ (Sumioka et al. 2005), this might have an influence on whether and how quickly AICD can be shuttled to the nucleus. Therefore, as described supra, the assumption was challenged that α-CTF and β-CTF derived AICD are shuttled with equal efficiency or preference to the nucleus to activate transcription. Instead, it was assumed that it might be possible for the APP-RIP pathway, which can head along both the amyloidogenic and the non-amyloidogenic process, to also attain two different nuclear signaling outcomes.

The disruption of normal endocytosis of APP by Dynamin, one of the physiologically interesting proteins from our MS experiments (Table 13), showed that there is a clear decrease in the number of cells that contain nuclei with spherical AICD/Tip60/Fe65 (AFT) spots. Similarly, RT-PCR and fluorescence microscopy experiments with pre-formed α- and 13-CTFs and α- and β-KD APP cleavage mutants all pointed in the direction of β-CTF playing a dominant role.

An important further indication that BACE-cleavage is more conducive to signaling by AICD is the fact that application of a dilution series of a specific inhibitor of the amyloidogenic processing pathway of APP (Abbenante et al. 2000) clearly results in a matching reduction of the number of cells that show formation of nuclear tripartite AFT spots. The impact of this finding is augmented by the fact that in various cell lines and primary neurons, β-CTF makes up only approximately 5-10% of all CTFs. That inhibiting the formation of this small fraction makes a readily detectable difference in translocation of AICD to the nucleus is thus a further sign that β-CTF plays an important role in AICD signaling. It also means that slight shifts in processivity in the relative formation of α-CTFs to β-CTFs may dramatically change the outcome of APP signaling.

### Implications

In summary, the present invention provides several lines of evidence suggesting a preference of nuclear AICD localization to transcriptionally active complexes for the amyloidogenic pathway.

sAPPα has beneficial properties as a neurotrophic factor (Mattson et al. 1993). Also, Aβ as an inhibitor of LTP (Klyubin et al. 2005), as a toxic molecule (Singer and Dewji 2006) and as the initiator of the amyloid cascade (Hardy and Higgins 1992), is widely regarded as the main culprit in the development of AD. However, while BACE-KO mice are viable, they do show subtle behavioral deficits in a test that assesses spatial working memory (Ohno et al. 2004) and the data of the present invention indicate a significant role of β-CTF in nuclear signaling. It would therefore seem that shifting drug research from γ-secretase inhibitors to β-secretase inhibitors in the expectation of reducing side-effects may have to take into account the possibility of altered gene expression even when “only” inhibiting β-secretase.

In summary, both, the MS data and the analyses of AICD-mediated signaling according to the present invention emphasize the importance of APP sorting and trafficking for APP processing and signaling. With APP present in synaptic vesicles, this would seem like an efficient way to make sure that sAPPα is produced where it is needed most, as a protectant against excitotoxicity (Mattson et al. 1993), for example. The involvement of Clathrin and Dynamin in endocytosis of APP obtains a new dimension if this influences not only production of Aβ but also nuclear signaling. Therefore, the identification of new interaction partners of APP and further elucidation of the RIP-mediated signaling of APP is successfully provided by the present invention. Thus, several new avenues of research are provided such as the cloning of cDNA of proteins from Table 7 into the pUKBK-C system of the present invention (supra) and to monitor in cell culture whether these proteins colocalize with APP and whether they have an effect on APP processing. It would be of great interest to find one protein to enhance Aβ production, e.g. through more rapid internalization (cf. (Carey et al. 2005)), as blocking such a protein would seem pharmacologically more feasible than activating a stabilizer of APP such as X11α.

Furthermore, the crossing of currently breeding homozygous APP-TAP-AICD mice with APP-KO mice (Li et al. 1996) may be useful to ideally arrive at a homozygous APP-TAP-AICD (+/+)/APP (−/−) mouse, as the entirety of proteins interacting with APP would be bound to the bait protein of the present invention for purification, while levels of transgenic APP would still be at close to physiological levels and thus not be expected to entail potential overexpression artifacts.

With the data provided by the present invention which suggest reduced transcriptional activity by AICD when β-CTF is downregulated, it would also be beneficial to inversely manipulate the system by raising the ratio of β-CTF to α-CTF. Therefore, two procedures may be useful, for example, the cloning of the Swedish double mutation into the pUKBK-C-APP-Citrine expression vector according to the present invention, which would result in transfected cells producing far more β-CTF than normal (Cai et al. 2001). According to the data of the present invention that ought to result in the nuclei of more cells showing AFT complexes. Analogously, reducing the amount of α-CTF by applying an α-Secretase inhibitor ought to have a similar effect. If this substantiates the current evidence for a greater role of β-CTF-derived AICD in nuclear signaling, it would finally be necessary to determine how the generation of sAPPα, with its neurotrophic benefits, could be reconciled with the transcription of AICD targets.

These and other embodiments are disclosed and encompassed by the following description of the present invention, including inter alia by reference a PHD thesis. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

The above disclosure generally describes the present invention. A more complete under-standing can be obtained by reference to the following detailed description and experiments which is provided herein for purposes of illustration only and is not intended to limit the scope of the invention.

### EXAMPLES

The Examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed. by Beers and Berkow (Merck & Co., Inc., 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11, (2001), 98-107.

### Example 1

### Nucleic Acid Based Techniques

### Chemically Competent Cells

2 ml overnight culture of DH5α cells were used to inoculate 100 ml LB-medium in which growth was allowed to take place at 37° C. until turbidity of OD₆₀₀=0.4 (log phase) was obtained. Cells were centrifuged, washed and incubated for 20 min under mixing on ice in 20 ml CCMB (Potassium acetate (10 mM), Glycerol (10% (w/v)), CaCl₂ (80 mM), MnCl₂ (20 mM), MgCl₂ (10 mM), pH 6.0, sterile filtration), centrifuged and resuspended in 5 ml CCMB. 50 μl Aliquots were shock-frozen in liquid nitrogen and stored at ±80° C. The method relies on a combination of calcium- and glycerol induced shock.

### Purification of DNA

### Plasmid Preparation

Two different kits were used for purification of plasmid DNA; for minipreps (2 ml cultures, for cloning and sequencing), lysozyme induced cell wall disruption was used (Eppendorf Fastprep kit, #955150619), while alkaline lysis was used for maxipreps (100 ml cultures, for mammalian cell transfection). Plasmid DNA for cell transfection was additionally purified by precipitation: 0.5 volumes Ammonium Acetate (7.5 M) and 3 volumes EtOH were added, the solution was vortexed, centrifuged at 20′000 g, washed with 70% EtOH, dried and the resulting pellet resuspended in water. Plasmid DNA was accepted as sufficiently pure when A260/A280 was higher than 1.8.

### Fragment Purification

Linear DNA fragments <10 kb were purified by gel extraction (Qiagen, #28706) or column purification (Sigma, #NA1020), according to the manufacturer's recommendations. Both kits use chaotropic salts such as guanidine hydrochloride (GuHCl) to allow DNA binding to matrix material made of silicon dioxide (silica) under dehydrating conditions.

### Polymerase Chain Reaction (PCR)

PCR allows rapid amplification of DNA flanked by known sequences. For analytical purposes only, such as mouse genotyping, the robust but not proofreading RedTaq polymerase (derived from Thermus aquaticus, Sigma, #D-8312) was used. For preparative purposes, the proofreading Pfu Turbo polymerase (from Pyrococcus furiosus, Stratagene, #600250) was used. Primers were always designed to obtain a basic (not salt-adjusted) annealing temperature (Tₘ) of 58-61° C. Tₘ was calculated using the empirically fitted formula for long oligonucleotides (www.nwfsc.noaa.gov/protocols/oligoTMcalc.html):

$${T}_{m} / C ° \approx 64.9 + 41 \cdot \frac{( Gcount + Ccount - 16.4 )}{Oligolength}$$

Default reaction mixture and cycling program as detailed below. For diagnostic PCR, 20 μl reactions with 30 amplification cycles were used. The apparatus used was a Perkin Elmer 9700 thermocycler.

TABLE 8
Default PCR reaction conditions
50 μl PCR reaction

| | | |
|---|---|---|
| 1 | μl | Pfu turbo polymerase (stock: |
| | | 2.5 U/μl) |
| 1 | μl | each primer (stock: 25 μM) |
| 0.5 | μl | dNTP (100 mM total) |
| 5 | μl | 10x buffer (incl. 2.5 mM |
| | | MgCl₂) |
| 1 | μl | 1/500 diluted plasmid from |
| | | miniprep or 1/1000 diluted |
| | | from midiprep (~200 pg) |
| 41.5 | μl | H₂O |

| Temperature | | |
|---|---|---|
| (° C.) | Time (min) | No. of cycles |
| 95 | 1:30 | 1 |
| 95 | 0:30 | 28 |
| 55 | 0:30 | |
| 72 | 1:00/2 kb | |
| 72 | 5:00 | 1 |
| 4 | ∞ | 1 |

### Restriction-Based Cloning

Where combination of two DNA fragments was required, to restriction based cloning (as opposed to the Gateway recombination based cloning system) was resorted:

### Insert Preparation

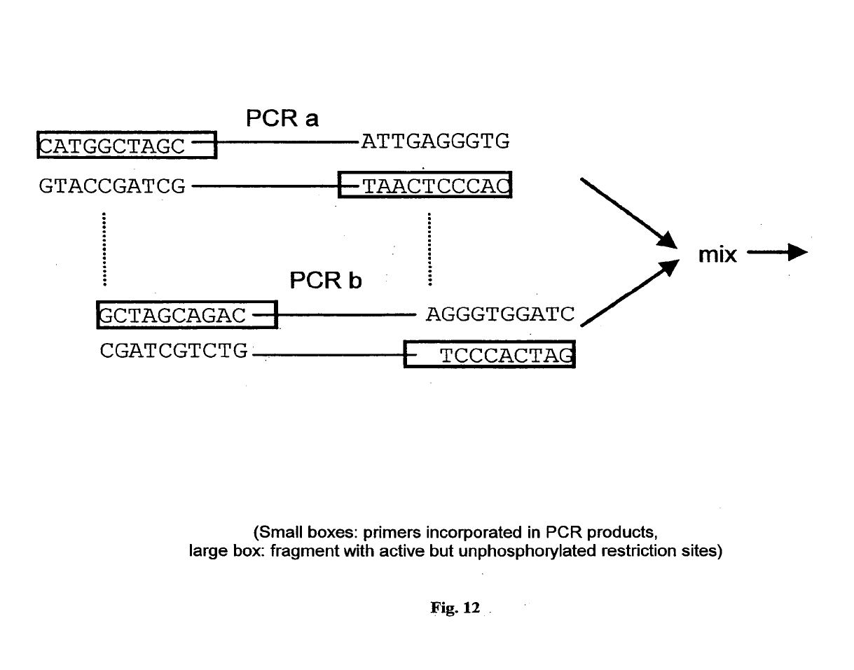

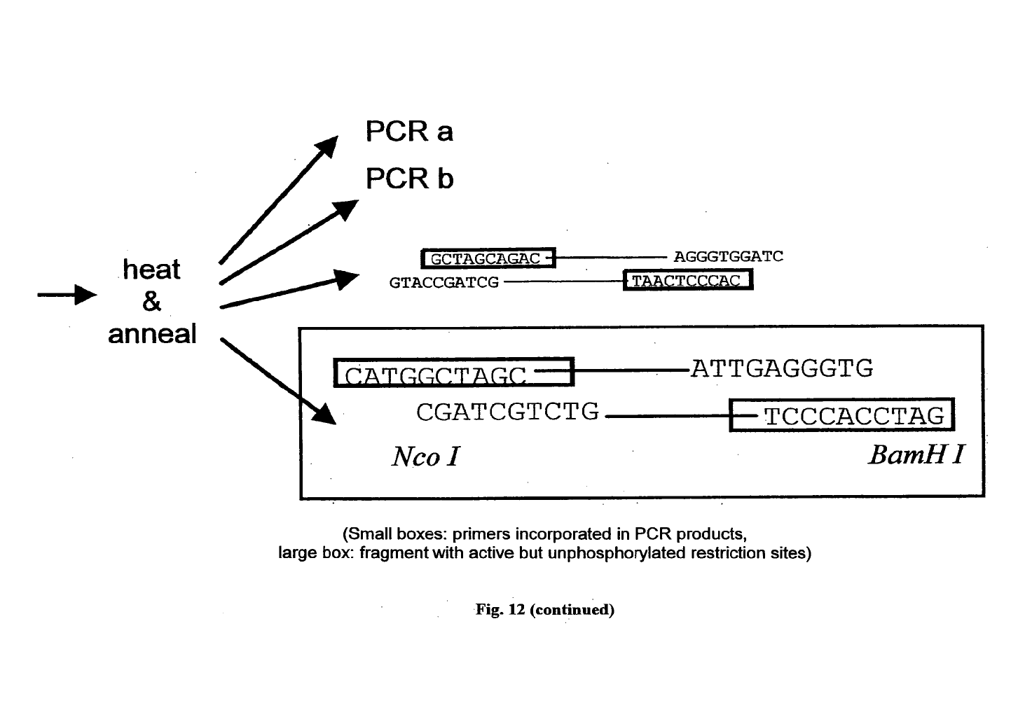

When the desired restriction sites were not already flanking the insert, inserts were PCR amplified using restriction site flanked primers. A minimum of 5 nt were added to the 5′ ends of primers for efficient enzyme cleavage. Where internal restriction sites were present, staggered PCR cloning was used to avoid the use of partial digestions, necessitating two separate PCRs according to the schematic representation as depicted in FIG. 12.

The two PCR products were mixed and reannealed into the four possible fragments by heating to 95° C. and cooling slowly to RT on a thermocycler, prior to phosphorylation with 1 μl T4 Polynucleotide Kinase for 30 min at 37° C.

### Restriction and Dephosphorylation

Reaction conditions were always chosen according to the manufacturer's protocol. Incubation periods generally were 1 h at 37°. Starting material was <1 μg for diagnostic and >1 μg for preparative purposes. Cloning procedures were unidirectional due to usage of two in-dependent, incompatible restriction sites—typically Sfi/AscI/Pme, with 8-12 by recognition sites, as implemented in the pUKBK vector system, described above. Plasmids were always de-phosphorylated by addition of 1 μl calf intestinal phosphatase (CIP) directly to the restricted plasmid and incubation for 30 min at 37° C.

### Ligation

For insertion of PCR-fragments into the plasmid backbone, T4 DNA ligase (Rapid ligation kit, Roche, # 11 635 379 001) was used to catalyze the covalent linkage of 5′-Phosphate groups with 3′-hydroxyl groups, using insert:plasmid ratios of around 5. Ligation was for 5 min at RT prior to transfection (described infra).

TABLE 9
Default ligation reaction with 5:1 insert to backbone ratio
20 μl rapid ligation reaction

| | | |
|---|---|---|
| 2 | μl | 5x DNA dilution buffer |
| x | μl | 1 part plasmid (~250 ng) |
| 8-x | μl | 5 parts insert |
| 10 | μl | 2x ligation buffer |
| 1 | μl | T4 DNA ligase (stock: 5 U/□l) |

### Site Directed Mutagenesis

For introduction of individual point mutations, site directed mutagenesis (SDM) was performed: mismatch primers containing the desired mutation flanked by a sufficient number of bases on each side to obtain a non salt-adjusted Tₘ of approximately 60° C. were used to prime a PCR reaction on plasmid DNA (final concentration of 20 pg/μl). PCR program: see supra, however with elongation temperatures of 68° for twice the usual time and only 19 cycles. 2 μl DpnI were added directly to the completed PCR reaction which was then incubated for 3 h at 37° C. for digestion of methylated (original, bacteria derived) plasmid prior to transformation.

### Transformation of Bacteria

Transformation of competent cells (described supra) occurred by adding 5 μl of ligation product or 1 μl of site directed mutagenesis product to one aliquot. Mixture was stirred, left on ice for 30 min, prior to performing a 1 min heat shock at 42° C., incubating the cells with 1 ml LB medium for 1 h at 37° C., centrifuging for 5 min at 3000 g and then plating the resuspended cell pellet on agar plates with the corresponding antibiotic.

### Plasmid Analysis

Plasmids were always verified prior to: further sub-cloning, storage-batch production of plasmid via maxiprep or cell transfection experiments.

### Restriction Analysis

Reactions were performed as appropriate and procedure as described in detail supra.

### Colony Screening PCR

Directly from agar plates, sterile plastic tips were used to inoculate miniprep cultures and then swirled in 60 μl H₂O, which was boiled at 95° C. for five minutes to break up cells and pelleted. 10 μl of supernatant were used as template in a 20 μl PCR. Diagnostic PCR program as described in detail supra.

### DNA Sequencing

Where PCR amplification of important components of a new plasmid was involved, these regions were sequenced by the Sanger sequencing method, using di-deoxy terminator nucleotides. This linear amplification (PCR with one primer only) method results in partial sequences of interest with a statistical fragment length distribution spreading over approximately 600 by that are separated in polymer-filled capillaries and detected by multiplexed fluorescence excitation of the dyes attached to the terminator nucleotides. BigDye reaction kit 1.1 (Applied Biosystems, #4336776) was used: 1 μg plasmid DNA, 25 μmol primer, 8 μl provided reaction mixture (containing polymerase and ddNTPs). Cycling parameters were as follows: 95° C.: 15 s, (95° C.: 30 s, 50° C.: 30 s, 60° C.: 4 min)×25. Reactions were precipitated by addition of 9 volumes 66% isopropanol and centrifugation for 15 min at 20′800 g and RT, which was repeated prior to resuspension in 10 μl H₂O.

### Realtime PCR (RT-PCR)

“Realtime” alludes to the fact that DNA intercalating dyes that strongly fluoroesce only when bound to double stranded DNA can be employed to monitor the growing number of PCR products during amplification; therefore, the term is synonymous with “quantitative”.

### RNA Extraction

One 6 cm cell culture dish was harvested for each experiment 24 h after transfection and/or treatment: Cells were washed once with PBS and lysed with 1 ml Trizol reagent (Invitrogen, #15596-018), a mono-phasic solution of guanidine isothiocyanate and phenol. Undissolved cell debris was pelleted at 12′000 g for 10 min. All centrifugation steps were performed at 4° C. ⅕ volume (0.2 ml) Chloroform was added for precipitation of proteins, while DNA remained in the interface between the organic and the aqueous phase wherein the RNA resided. With the aqueous phase, the RNA was extracted and subjected to two final wash steps in 75% EtOH in DEPC treated H₂O before drying and redissolving the RNA in DEPC treated H₂O. RNA was stored at −80° C.

cDNA Synthesis

RNA was reverse transcribed using oligo-dT primers targeting the poly-A tail of mRNA and SuperScript viral reverse transcriptase (Invitrogen, #12371-019). RNA concentrations were measured photometrically at 260 nm and 2.5 μg from each sample were used for cDNA synthesis: reaction mixtures contained 1 mM dNTPs, 50 ng/μl oligo-dT₁₂₋₁₈ primers and 2.5 μg RNA in DEPC-treated H₂O. After RNA denaturation at 65° C. for 5 min, 1×RT buffer was added, as well as 6 mM MgCl₂, 10 mM DTT and RNAse inhibitor (40 U). SuperScript (50 U) was added and reverse transcription took place at 42° C. for 50 min. Samples were heated to 70° C. for 15 min, cooled and RNAse H (2 U) enzyme added for 20 min at 37° C. to degrade the RNA in RNA/DNA hybrids.

### RT-PCR Reaction and Data Analysis

For quantitative PCR, it was relied on the SybrGreen dye that binds indiscriminately to double-stranded DNA (dsDNA), fluorescing intensely only in the dsDNA-bound state. This chemistry is generic and thus compatible to using normal oligonucleotides as primers for each gene. Primers were designed to be of similar Tₘ and to yield products of slightly over 100 by length. 25 μl reactions contained 0.5 μM primers, 12.5 μl 12× SybrGreen reaction buffer (Stratagene, #929581) and cDNA transcribed from initially 20 ng RNA. 40 annealing/extension cycles were run on the ABI Prism 7700 Sequence detector.

When semiquantitatively comparing the expression of a single gene in different samples, 100% amplification efficiency was assumed, i.e. a doubling of product with each cycle inside the exponential phase of the PCR, as efficiency considerations do not play a role when looking to see only whether a gene is up- or down-regulated under certain treatment conditions. An average Ct value was calculated for two reference genes in each sample as well as the relative amounts of all other genes were calculated in accordance with:

Abundance(cDNA)/Abundance(cDNA_{reference})=2^{(Ct}^{_{reference}}^{-Ct)}

Up- or down-regulation of individual genes in between samples was measured by dividing the above relative abundance of these cDNAs from two different samples and performing the non-parametric Mann-Whitney U test with n>3 to test for significance at the p=0.05 level.

### Example 2

### Protein Biochemistry

Protein Extraction from Cell Culture

For analytical IP of transfection based experiments, single 10 cm dishes were used for sample preparation. For TAP experiments using stably transfected cell-lines, twenty 15 cm dishes were used for each pull-down experiment. Medium was discarded and the cells were washed carefully once with cold PBS. Lysis occurred using cytoMgCa buffer for IPs or SBB lysis buffer with 1% TX-100 for TAP experiments, using 1 ml per 10 cm dish and 2.5 ml per 15 cm dish, respectively. Lysed cells were scraped from dishes and incubated for 10 min on ice prior to a freeze-thaw cycle at −80° C. Cell debris, nuclei and unsolubilized membranes were then pelleted at 20′800 g at 4° C. for 20 min and discarded, while the supernatant was used for further analysis.

Protein Extraction from Mouse Brains

### Homogenization

Mouse brain homogenization always was performed in a Potter homogenizer, using 5 ml glass homogenization tubes fitted with Teflon pestles, under cooled conditions. 15 strokes at 600 rpm were used to disrupt and shear brain tissue and cell membranes. Cerebellum was always cut away using a scalpel, and discarded.

For synaptosome preparation, homogenization occurred in 4 ml of SynaptosomePrep buffer A (0.32 M sucrose, 10 mM HEPES, 1 mM MgCl₂, 0.5 mM CaCl₂, Complete Proteinase Inhibitor Cocktail (Roche) 1 tablet/50 ml, pH 7.4), for TAP purifications, 4 ml SBB lysis buffer (NP40 or Triton X100; 0.4%-1%; TrisHCl 40 mM, KCl 150 mM, 5% glycerol, Complete Proteinase Inhibitor Cocktail (Roche) 1 tablet/50 ml, β-ME 5 mM, pH=7.4) were employed per brain, followed by centrifugation at 4° C. for 20 min at 20′800 g to separate cytosolic and dissolved membrane components from insoluble membrane components and nuclei.

### Synaptosome Preparation

During the entire procedure, samples were kept at 4° C. Brain homogenate was centrifuged for 10 min at 1000 g, pelleting nuclei, and the supernatant was retained. The pellet was washed once with SynaptosomePrep buffer A (buffer A) by rehomogenizing and then centrifuged for 10 min at 1000 g. The combined supernatants were centrifuged for 15 min at 20′800 g, yielding the crude membrane fraction which was washed in 1 ml buffer A by resuspension and centrifuged again at 20′800 g for 10 min and finally resuspended in 0.5 ml buffer A. This suspension was then centrifuged for 120 min at 100′000 g in an ultracentrifuge on a sucrose step gradient consisting in equal parts of 1.2 M, 1 M and 0.85 M sucrose 10 mM HEPES, pH 7.4 buffer. Synaptosomal plasma membranes forming a thin band between the 1.0 and 1.2 M sucrose phase were collected. The aspirated samples were diluted 5 times with 1× cytoMgCa buffer (140 mM KCl, 12 mM NaCl, 5 mM MgCl₂, 2 mM CaCl₂, pH 7.4) and recentrifuged at 48′200 g. For lysis, this pellet was finally resuspended in 200 μl cytoMgCa buffer containing 1% TX100. This lysate was directly added to prepared affinity purification beads, described infra.

### Enrichment of Nuclei

For enrichment of nuclei from mouse brain homogenate as described supra, the centrifugation step at 20′800 g was not performed. Instead, after homogenization, filtration followed, using nylon inserts (100 μm) for 50 ml Falcon tubes and 100 g for 1-2 min. This filtered homogenate was centrifuged in 2 ml Eppendorfs, using 600 g for 10 min. The supernatant was discarded, the pellets resuspended (using a 1 ml Gilson pipette) in 1.5 ml homogenization buffer. Re-centrifugation followed, as above, and again the supernatant was discarded. This nucleus-enriched fraction preparation procedure was shown to be effective by Histone staining in WB.

### Protein Concentration Determination

A Lowry (Lowry et al. 1951) based protein assay (BioRad, #500-0114) was used to determine protein concentrations from homogenates. It is a two-step reaction where under alkaline conditions proteins present in the sample first reduce copper, which in turn reduces Folin reagent, resulting in light absorption that was measured at 595 nm. A standard curve was always prepared with 4 dilutions of BSA in the current lysis buffer, at concentrations ranging from 0.6 μg/ml to 5 mg/ml.

### Protein Purification Techniques

Purification of AICD from E. coli

His₆-AICD or AICD-His₆ transfected IPTG-induced BL21 DE3 E. coli (Novagen, #693879) were harvested by centrifugation at 4′000 g. Aiming to obtain an OD₆₀₀=30 based on the final cell density of the expression culture, E. coli cells were resuspended in EC resuspension buffer (50 mM NaPi, 10% glycerol, 300 mM NaCl, 200 mM imidazole, pH 7.0). The addition of TX-100 to 1% solubilized the lipid membrane during a 10 minute incubation on ice, permitting access of the peptidoglycan layer to Lysozyme, at a final amount of 1 mg/ml. Complete lysis and shearing of DNA was carried out by sonication at medium output levels and a 30% duty cycle until DNA-caused viscosity disappeared. Cell debris was pelleted at 20′000 g for 20 min at 4° C. Affinity purification of His₆-tagged AICD was performed using Ni-NTA gravity flow columns (Qiagen, #30622) according to the manufacturer's recommendations.

### Immunoprecipitation (IP)

Resins for IPs were prepared using 60 μl Protein G sepharose (Amersham, #17-0618-01), plus 2 μg antibody, and 0.2% TX-100 in 0.5 ml PBS. After 2 h incubation on a rotating wheel at 4° C., resins were washed 3 times with 0.75 ml PBS. Solubilized proteins were first precleared by incubation with equal amounts of washed but not antibody-coupled resins and were then added (300 μl per IP, typically in the range of 2-5 mg/ml) and incubated with the prepared antibody-coupled resins overnight at 4° C. on a rotating wheel. Three 0.75 ml PBS washing steps preceded elution using 50 μl of 1 M acetic acid and finally 50 μl SDS sample buffer (450 mM Tris-HCl, 12% (w/v) glycerol, 4% SDS, 7.5‰ Coomassie blue, 5% β-ME, pH 8.45), resulting in two separate eluate fractions.

### Synthetic Bait Peptide Based Purifications

Biotinylated peptides were custom-ordered from Metabion in an N-terminally Biotinylated form, including their in-house hydrophylic chemical linker between the Biotin moiety and the peptide. All incubations were performed at 4° C., and magnetic bead pull-downs in a permanent-magnet holder. For each experiment, 100 μl Streptavidin-coated Dynabeads M280 (Dynal, #112.06) were washed twice with PBS in order to get rid of azide. These magnetic beads were then incubated for 2 h with 500 μl of a 0.1 mg/ml solution of Biotinylated bait peptide or the mutant thereof as a negative control, prior to washing away unbound peptide with three PBS-washes. Typically, the lysate from half a mouse brain or from five 15 cm cell culture plates—less for WB only, with appropriately scaled use of matrix—was applied to washed beads without bound peptide as a preclearing step and the supernatant from this step was then added to the bait peptide coupled Dynabeads. The duration of this final incubation was 2 h on a rotating wheel. Prior to elution, the beads were washed thrice with PBS. For the AICD(wt)/(mut) peptides (described infra), proteins were eluted using 1 M GuHCl, unless noted otherwise. For the PrSciAICD(+/−) peptides (described infra), a more specific procedure was used: after an additional washing step in PreScission buffer (50 mM TrisHCl, 2.5 mM EDTA, 1 mM DTT, 2 mM NaCl, pH 7.0), 1 μl PreScission protease was added to the bait/interactor assembly and cleavage was allowed to take place at 10° C. for 2-4 h. PreScission enzyme, fused to a GST moiety, was captured and removed from the sample by binding to glutathione-sepharose 4B beads that bind GST (Amersham #17-0756-01).

### Tandem Affinity Purification Method (TAP)

The starting point for this procedure was always cell or mouse brain homogenate prepared as detailed supra. In the following, all centrifugations of solutions containing resin occurred at 1500 g, for 5 min at 4° C. Similarly, all incubations of solutions containing resin occurred at 4° C. on a rotating wheel, enabling thorough mixing of the samples.

Preclearing step: For each purification sample, 2 slurries containing 1 ml Sepharose CL4B (Sigma #CL4B200) per mouse brain or per twenty 15 cm cell culture dishes, were pre-equilibrated twice with 10 volumes SBB lysis buffer (10 ml), entailing repeated resuspension and centrifugation prior to finally discarding supernatant equilibration buffer. Volume adjusted equal total protein amounts (see supra) of each sample were incubated with the washed resin for at least 30 min. Samples were centrifuged and the incubation was repeated with the non-bound supernatant (SN) and the second prepared resin.

600 μl Streptavidin Sepharose CL4B resin (Novagen, #69203) per mouse brain or per twenty 15 cm cell culture dishes were preequilibrated twice (see above) with 20 volumes (12 ml) SWB (same buffer as SBB lysis, but without Proteinase inhibitor cocktail, and only 0.4% of detergent). According to the manufacturer's data, 1 ml resin could theoretically bind ca. 1.6 mg of NTAP-AICD (14 kDa). The SN from the preceding preclearing steps were incubated with the thus prepared resin for at least 2 h.

Samples were then centrifuged, and the SN discarded after re-centrifugation to capture any additional resin, minus a 25 μl aliquot for WB/silver staining (ss). Using 20 volumes of SWB each time, the resins were washed 3 times. Resuspended resins were always gently inverted several times prior to centrifugation. After the final centrifugation step, 1 ml SEB (same buffer as SWB+2 mM Biotin, NH₄HCO₃ instead of TrisHCl) was added to each resin and the mixtures incubated for an elution duration of at least 1 h.

For single affinity purification, as finally used for LTQ (—FT) and MALDI-TOF/TOF analysis (as described in detail infra), this elution slurry was centrifuged and the SN (=eluate 1; EL1) evaporated by vacuum pump. For TAP, as used for analysis by gel extraction followed by LCQ analysis, the second purification step occurred as follows. 200 p. 1 Calmodulin resin (Stratagene, #214303-52) were pre-equilibrated twice with 10 ml CBB and resuspended in CBB, using triple the EL1 volume (3 ml). EL1 was added to the resuspended resin and the slurry was incubated for at least 2 h, centrifuged and the non-bound SN discarded, minus a 25 μl aliquot for analysis. Three washing steps with 10 ml CBB (CXB, 100 mM CaCl₂, Complete Proteinase Inhibitor Cocktail (Roche) 1 tablet/50 ml, β-ME 10 mM) each ensued. After centrifuging and discarding the wash buffer for the third time, the proteins bound during the second affinity purification step were first eluted (incubation=1 h) using CEB (CXB, 100 mM CaCl₂, Complete Proteinase Inhibitor Cocktail (Roche) 1 tablet/50 ml, β-ME 10 mM) and then PAGE loading buffer under cooking at 95° C. for 5 min).

### SELDI-TOF

Measurements were performed on the ProteinChip Reader (Series PBSII from Ciphergen), using either of two chip types: PS20 chips with epoxide chemistry for covalently binding free amine groups (Ciphergen, #C553-0045) or NP20 chips with inert silicate coating (Ciphergen, #C573-0043). All binding and washing reactions were performed according to the manufacturer's protocols, using 2 mg Extravidin (Sigma, #E2511) and 500 ng bioAICD biotinylated peptide (as described infra) per spot for binding of lysate.

### SDS-PAGE (1DGE)

For size based separation of denatured proteins, sodium dodecyl sulfate polyacrylamide gel electrophoresis was used; SDS covers the proteins with a negative charge density of 1/aa by binding with its hydrocarbon chain to the polypeptide backbone through van-der-Waals interactions, rendering gel migration in a homogenous electric field largely size dependent. For one-dimensional gel electrophoresis, it was adhered to the original recipe (Laemmli 1970) for 10% Tris-Glycine gels, gradient gels were used (10-20% Tricine gels, Invitrogen, #EC66252BOX) for enhanced resolution, according to the manufacturer's recommendations. In both cases, sample preparation involved simultaneous denaturation and reduction of proteins in SDS sample buffer for 5 min at 95° C.

### 2DGE

### Isoelectric Focusing (IEF)

60 μl of samples from synthetic bait peptide mediated pull-down of mouse brain homogenate were mixed by vortexing in 300 μl Rehydration Buffer (8.5 M Urea, 4% CHAPS, 0.5% pharmalytes pH 3-10, 1.2% DeStreak reagent) containing pharmalytes and DeStreak reagent (Amersham, #17-0456-01 and #17-6003-18, respectively). Samples were incubated therein at RT for 30 min and then centrifuged at 20′800 g for 10 min. For passive rehydration, samples were then loaded into a tray on 7 cm IPG strips (pH 3-10, Bio-Rad, #163-2002) for 15 h at 20° C. Strips were transferred into an IEF tray and focused in a Bio-Rad Protean IEF Cell using a stepwise—1 h each—increase in voltage from 150 V, 300 V, 500 V, 1 kV to 10 kV, finishing with 60 kVh at 10 kV. Before loading for SDS-PAGE, the IPG strips were equilibrated in 2DGE-equilibration buffer (2% DTT, 6 M Urea, 2% SDS, 50 mM Tris-Base pH 8.8, 20% glycerol) for 30 min and then again in the same buffer but replacing the DTT with 2.5% iodoacetamide for cysteine methylation.

### (i) Gel Electrophoresis

Gels were prepared as 12% acrylamide solutions in 2D Gel Buffer (0.375 M Tris-Base, pH 8.8, 5% glycerol and 1% SDS), with 0.05% APS and 0.05% TEMED inducing polymerization. IPG strips were fixed at the top of the gels in 2D Running Buffer (25 mM Tris-Base, 0.2 M Glycine, 0.1% SDS) containing 0.5 agarose. The gel was run under constant cooling at 200 V constant and 500 mA maximum current. Staining was performed as described infra.

### Silver Staining (ss)

A silver staining protocol was used that does not involve using glutaraldehyde, and which is thus MS-compatible. ssFix (50% MetOH, 12% acetic acid, 0.05% Formalin (35% formaldehyde)), ssStain (0.2% AgNO₃, 0.076% formalin), ssDevelop (6% Na₂CO₃, 0.05% formalin, 0.0004% Na₂SO₃) and ssStop (50% MetOH, 12% acetic acid) buffers. All steps were carried out at RT. Gels were fixed in ssFix for at least 1 h or over night, washed thrice in 35% EtOH for 20 min, sensitized in 0.02% Na₂SO₃ for 2 min, rinsed in H₂O three times for 5 min each, stained for 20 min in ssStain, washed twice for 1 min in H₂O, developed in ssDevelop until the desired exposure was obtained and the reaction stopped with ssStop solution.

### Western Blotting (WB)

Western Blotting allows identification of individual proteins in a mixture, assuming a specific antibody is available, or when a generic tag has been genetically fused to the protein of interest. It further allows visualization of protein processing based on the fact that proteins are size-separated in a first step by 1DGE (see supra). Gels were equilibrated in WB transfer buffer for 5 min and then used to form a sandwich cassette consisting of following pre-wetted layers, from cathode to anode: sponge, filter paper, gel, Protran Nitrocellulose transfer membrane (0.1 μm, Schleicher & Schuell, #10402096), filter paper, sponge. Electrophoretic transfer was set to 1 h at 90 V constant, at 4° C. with precooled WB transfer buffer.

The nitrocellulose membrane was then blocked for 30 mM with PBS containing 5% milk powder (blocking buffer), incubated with blocking buffer containing the primary antibody at the manufacturer's recommended dilution for a minimum of 1 h, washed thrice with PBS for 5 mM each, incubated again for at least 1 h in blocking buffer containing the 1° antibody species directed horse-radish-peroxidase conjugated (HRP) secondary antibody at a 1:4000 dilution, washed again and developed using commercial electrochemiluminescent reagents (ECL, Pierce, # 34095). ECL was captured on X-omat LS film (Kodak, #868 8681) developed in a Kodak X-OMAT 2000 processor.

### Example 3

### Mass Spectrometry

### Sample Preparation

### Direct In-Solution Preparation

Samples from affinity purifications (cf. supra) were evaporated down to volumes of approximately 90-100 μl (EL1). Acetone precipitation was performed by adding 600 μl (6 volumes) of −20° C. prechilled Acetone. Samples were inversed 5 times, centrifuged and incubated at −20° C. for 4 h. Precipitate was pelleted by centrifuging at 20′000 g at 4° C. for 10 min. Any KCl still present from the TAP purification is not eliminated in this step, does however not inhibit Trypsin and is lost during the C₁₈ purification described infra. Pellets were resuspended by vortexing in 50 μl of reduction & alkylation buffer (70% H₂O, 10% TCEP, 10% Rapigest (10 mg/ml; 1% in 50 mM NH₄HCO₃, pH 7.8), 100 mM NH₄HCO₃ solution, pH 7.8) containing TCEP reducing agent (e.g. from iTRAQ kit, Applied Biosystems, #4352135) and Rapigest acid-cleavable detergent (Waters, #186001861) for enhanced trypsinization. 2 μl aliquots were saved for ss analysis (predigested sample). Samples were reduced for 1 h at 60° C., under mild shaking (300 rpm). 3 μl MMTS blocking reagent each (e.g. from iTRAQ kit) was added, samples were mixed and incubated for 10 min at RT, resulting in quantitative methylation of reduced Cystein residues. For trypsinization, a 1:100 Lys-C (cuts only at K but is more robust than Trypsin) and Trypsin to protein ratio (w/w) was used, based on calculated protein amounts from densitometry of ss-1DGE. A 1 h 30 min Lys-C digestion at 37° C. preceded Tryptic digestion due to the robustness of the enzyme. Sample volumes were then raised to 300 μl by addition of a 100 mM Ammonium bicarbonate (pH 8.0) solution. This volume increase was used so that KCl from the elution buffer or other salts would be diluted. Up to 3 μl of 0.5 ug/μl Trypsin solution (1:100) were then added for overnight digestion at 37° C. on a shaker under slow shaking (300 rpm). The degree of digestion was monitored by ss-1DGE (supra)—undigested and digested sample at similar concentrations loaded next to each other for comparison. The acid-labile Rapigest detergent had to be degraded prior to C₁₈ purification: samples were acidified to pH=1.0 by 20 mM HCl (verified on pH paper), heated at 37° C. for 30 min and centrifuged 20′800 g for 10 mM at RT. The resulting SN was used for C₁₈ purification prior to running the samples on an LCQ/LTQ.

### (i) C₁₈ Microspin Column Peptide Purification

Microspin C₁₈ columns (Harvard, #74-7206) were used to desalt unlabeled tryptic peptides. All centrifugations were for 2 min, at 1000 g and RT. Columns were wetted with 100 μl 100% acetonitrile (ACN) before equilibration with 100 μl H₂O. The 300 μl samples from tryptic digestion were acidified with enough 10% formic acid (FA) to obtain a pH of 2.5, as checked on pH paper (0.1% FA solution corresponds to ca. pH=2.5, for comparison), typically requiring 40 μl 10% FA. This sample was loaded on the columns and the flow-through was reloaded an additional 2 times. Salt was washed out with a triple application of 100 μl of 0.1% FA. Final elution occurred in 200 μl 80% ACN, 0.1% FA in H₂O. The eluate had to be evaporated down to 40 μl at least, as less volume reduction would not have met the requirement of getting rid of the 80% ACN in the 200 μl elution buffer volume, which would have resulted in the peptide material not binding to the reverse phase column fractioning the tryptic peptides on the LCQ/LTQ.

iTRAQ Labeling

Importantly, this labeling procedure was only used in conjunction with already trypsinized proteins, as the reagents employed are amine-specific and thus are far more efficient (i.e. the probability of deriving quantitative information on a protein is higher) when peptides are treated. Based on an average of LTQ base peak ion and total ion counts, equivalent total amounts of peptides from the samples to be compared were dried down and redissolved in 15 μl iTRAQ redissolution buffer (iTRAQ kit, Applied Biosystems, #4352135). Negative control samples were labeled with the 114 Da reporter, and the positive sample with the 116 Da reporter, allowing best possible quantification resolution for individual duplex reactions with the four possible reagents (114.1-117.1 Da). 70 μl of EtOH was added to each reagent vial for prevention of hydrolysis and 35 μl thereof to each digest tube. Samples were vortexed, centrifuged and incubated for 1 h at RT, shaking at 300 rpm. 1 volume of H₂O (50 μl) was added for hydrolysis of excess reagent during a 30 min incubation at RT, whereupon the two samples were pooled and dried down to approximately 10 μl.

### (i) C₁₈ Ziptip Purification

10% trifluoroacetic acid (TFA) was added to make samples acidic (pH=2.5) for the Ziptip extraction, as checked on pH paper. 10 μl Ziptips C₁₈ (Millipore, #ZTC18S096) were employed using 20 μl pipettes, allowing aspiration of 20 μl. Tips were wetted twice with ACN before equilibrating twice with 0.1% TFA. Peptides were then bound by aspirating and dispensing 10 times each, salts were washed out thrice with 0.1% TFA. Elution occurred in two steps: first by aspirating and dispensing 5 times each in 8 μl of a 50% ACN, 0.1% TFA solution and second by repeating this procedure in 8 μl 80% ACN, 0.1% TFA. Binding, washing and elution were repeated three times for each sample and all eluates were finally combined, dried down and resuspended in 0.1% TFA for spotting on MALDI plates.

### Gel Excision Peptide Extraction

For analysis by LCQ-MS/MS, bands from MS-compatible ss gels were excised with a scalpel and processed entirely according to the Montage in Gel Digestion kit (Millipore, #LSKGDZP96) according to the manufacturer's protocol.

### LCQ/LTO LC-MS/MS

Tandem MS spectra were collected on Finnigan LCQ Deca or Finnigan LTQ (−FT) machines. Prior to electrospray ionization, samples in 0.1% FA were separated online in a reverse phase microcapillary column in an ACN gradient. The resulting .dta files were converted to .mzXML format (Pedrioli et al. 2004) and channeled into the Trans Proteomic data analysis Pipeline (TPP, infra).

### LC/MALDI-TOF/TOF

MS of iTRAQ labeled samples was performed on an Applied Biosystems 4800 (AB 4800) vertical MALDI-TOF/TOF. These samples cannot be analyzed on an LCQ, LTQ, or even LTQ-FT, as the reporter masses (<118 Da) are below the full MS scan m/z range limits on these instruments.

The MALDI plate was prepared for sample spotting by washing with H₂O and conventional dishwashing detergent using a toothbrush, cleaning with Kimwipes, wiping with isopropanol and finally applying conventional metal polish solution for increased plate hydrophobicity, rubbing the surface until it was shiny.

Peptide separation and spotting was performed in a linear ACN gradient on a Dionex UltiMate LC system with a 70 μm diameter reverse phase column. Eluting fractions were mixed with CHCA matrix solution (2.5 mg/ml CHCA in 70% ACN, 0.1% TFA) and deposited on the MALDI plate by a Dionex Probot spotting device.

The UV-trace recorded during the offline-LC was correlated to the spot number and the resulting spots analyzed on an AB 4800 after calibrating m/z using the 4 on-chip calibration positions containing GluFib peptide calibrant. The following settings were used for data-dependent tandem MS, with laser shots always randomly dispersed: 1000 shots per full MS scan, minimum signal-to-noise ratio (S/N) for MS/MS=75, MS/MS-fragmentation energy: 1 kV, maximum number of collision induced dissociations (CID) per fraction=12. For the centroid conversion, i.e. the peak data extraction into the text file used for the Mascot search, the parameters were set at: minimum S/N=10, maximum number of peaks per precursor=50, 5 peaks maximum per 200 Da and precursor mass minus 20 Da is the upper limit. This latter parameter excludes any unfragmented precursor ion in the MS/MS that mustn't be counted as a genuine CID fragment.

### Database Searches and Analyses

### Transproteomic Pipeline (TPP)

For LCQ, LTQ and LTQ-FT derived data, Sequest/Comet, PeptideProphet and ProteinProphet have been fused into a single software suite (Institute of Systems Biology, Seattle) that allows: scoring of peptide matches to CID spectra by Sequest or Comet (Yates et al. 1995) and absolute assignments to the probability that a specific peptide or even protein was present in the analyzed sample by PeptideProphet (Keller et al. 2002) and ProteinProphet (Nesvizhskii et al. 2003), respectively. Briefly, the theory behind absolute probability assignment is as follows: The underlying assumption is that the quality of spectra inside the two distinct populations of correctly and incorrectly identified peptides or proteins is normally distributed (Gaussian). Plotting the peptide or protein score distribution yields a discrete curve to which two Gaussians are optimally fitted. Denoting the probability that an ID with the score D belongs to the correctly identified population as p(+|D) and the probability that an ID in the correctly identified group has the score D as p(D|+), the former can be calculated as:

$$p ( + \vert D ) = \frac{p ( D \vert + ) \cdot p ( + )}{p ( D \vert - ) \cdot p ( - ) + p ( D \vert + ) \cdot p ( + )}$$

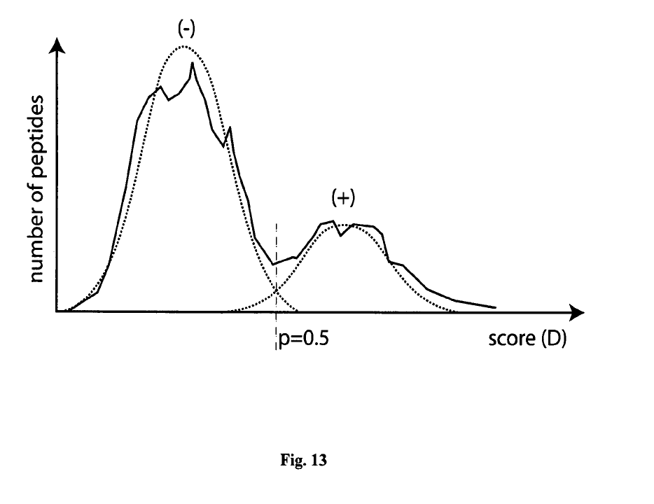

The total area under the curve corresponds to p(+)+p(−)=1, allowing normalization of each sample; see FIG. 13.

Comet peptide search parameters are given in detail in table 10 below.

TABLE 10
Comet search parameters used for the identification of proteins based on
the CID, i.e. tandem MS spectra of individual peptide precursor ions

| Comet Parameter | Setting |
|---|---|
| Database | IPI |
| Taxonomy | mus musculus |
| Enzyme | Trypsin |
| Missed cleavages | 1 |
| Number of correct termini | 2 |
| Static modifications | MMTS (C), (iTRAQ (N-term), iTRAQ |
| | (K), as applicable) |
| Peptide charge | 2+ |
| Mass tolerances | 3.0 Da |
| Peaks | average mass |

“missed cleavages” denotes the number of internal Arginines or Lysines in an identified peptide that are not immediately followed by Proline. The number of correct termini is used by PeptideProphet to score IDs but can also be set to 2 for higher stringency. Static modifications are used to denote quantitative changes to aa, while using dynamic modifications can be used to allow a certain degree of missed reactions but raises search time disproportionately and may result in more false IDs.

### Mascot

MALDI-TOF/TOF data was processed with Mascot, as Sequest, PeptideProphet and ProteinProphet have not yet been optimized for specific MALDI ionization and spectra characteristics, using score calculations that have been trained on LCQ and LTQ datasets. Settings are depicted below as entered into the MS/MS-ion search form. The data-file is a peak-list prepared as described supra or can be formed by concatenating .dta files generated on LCQ/LTQ apparatus by Sequest.

TABLE 11
Mascot search parameters used for MALDI-TOF/TOF data
for comments, confer Table 10

| Mascot Parameter | Setting |
|---|---|
| Database | SwissProt |
| Taxonomy | mus musculus |
| Enzyme | Trypsin |
| Missed cleavages | 2 |
| Static modifications | iTRAQ (N-term), iTRAQ (K), MMTS (C) |
| Dynamic modifications | Oxidation (M) |
| Peptide charge | 1+ |
| Mass tolerances | Default |
| Machine | MALDI-TOF/TOF |
| Peaks | Monoisotopic |
| File format | Mascot generic |

### Example 4

### Cell Culture

### Hek 293 Cells

Human embryonic kidney cells (Deutsche Sammlung von Mikroorganismen and Zellkulturen, DSMZ ACC 305) were cultivated at 37° C., 5% CO₂, 95% humidity in Dulbeccos modified eagle medium (DMEM, Invitrogen #52100-039) supplemented with 10% fetal calf serum (FCS) and Penicillin/Streptomycin (PS, Invitrogen #10378-016). For passaging, cells were loosened from plates by strong pipetting of fresh medium on cells and dispersed by several aspiration and dispensing cycles. Cells were stored in 5% dimethylsulfoxide (DMSO) and 50% FCS at −80° C. After thawing at 37° C., cells were centrifuged for 2 min at 1000 g and resuspended in fresh medium in order to rid the medium of DMSO.

### SH-SY5Y Cells

SH-SY5Y neuroblastoma cells (Deutsche Sammlung von Mikroorganismen and Zellkulturen, DSMZ #ACC 209) were cultivated at 37° C., 5% CO₂, 95% humidity in DMEM nutrient mix F-12 (Invitrogen, #32500-035) supplemented with 20% FCS and PS. For passaging, cells were scraped from plates and dispersed by several aspiration and dispensing cycles in fresh medium. The maximum splitting ratio was 1:5. Cells were stored in 5% DMSO and 95% medium at −80° C. After thawing at 37° C., cells were centrifuged for 2 min at 1000 g and resuspended with a flame polished Pasteur pipette in fresh medium.

For differentiation of SH-SY5Y cells into manifesting a neuronal phenotype, cells were seeded onto collagen type I coated dishes or glass slides at a density of 2×10⁴ cells/cm² and treated with 20 μM retinoic acid (RA) for five days.

### Transfection

Cells were transfected when 70-80% confluent, ideally passaged the day before transfection, reducing any extracellular matrix deposition surrounding the cells hindering transfection. Lipofectamine 2000 (Invitrogen, #11668-019) was used in a 2:1 v/w ratio in regard to plasmid DNA (Example 1). For 10 cm diameter cell culture plates, 30 μg DNA was used, 10 μg for 6 cm plates and 3 μg per well on slides. LF and DNA were mixed separately in 50 times the LF volume of Optimem with Glutamax (Invitrogen, #51985-026), incubated for 5 min at RT prior to mixing and DNA/LF complex formation at RT for 20 min prior to careful pipetting onto cell cultures. Transfection medium was replaced after 2 h with fresh medium supplemented with any inducers or secretase inhibitors as required by the current experiment. Unless otherwise noted, experiments were halted 24 h after transfection.

### Stable Cell Lines

When required for protein expression prior to selection, transcription was induced by Tebufenozide (TEB) activation of cDNA in the pTBoris system (Von Rotz et al. 2004). Cells transfected with fluorescent proteins were picked with sterile 20 μl tips from open plates under the fluorescence microscope and transferred to 12 well plates. Enriched fluorescence colonies formed in these wells were selected again until the desired degree of transfected cells was stable.

Where proteins were not tagged with a fluorescence protein (cf. pNTAP-AICD) for experimental reasons, cells containing the construct had to be selected by use of negative selective pressure in the form of the antibiotic Geneticin (G418, Invitrogen, #10131-027), at final concentrations of 250 μg/ml. The regime was started 3 days after transfection and maintained continuously.

### Example 5

### Immunocytochemistry

Cells were grown on fibronectin coated glass slides and transfected 24 hours prior to fixation, unless expressly stated otherwise. All the following incubations occurred under mild horizontal shaking at RT. The cells were washed with PBS (10×TBS: 0.84 M Tris-HCl, 0.16 M Tris-Base, 1.5 M NaCl) and fixed for 20 min, with 4% paraformaldehyde (PFA). They were then washed thrice for 10 min each with TBS (10×TBS: 0.84 M Tris-HCl, 0.16 M Tris-Base, 1.5 M NaCl) containing 0.05% Triton X-100 (TX100), before blocking unspecific antibody binding with TBS containing 0.02% TX100 and 10% horse serum for a minimum of 2 h. The first antibody was then applied in fresh blocking solution over night at 4° C. at the manufacturer's recommended concentration. Cells were then again washed and blocked as described above, whereupon the secondary dye-conjugated antibody was applied at concentrations of 1:250 for a minimum of 2 h. With the first ensuing washing step, DAPI nucleic acid UV-detectable stain was employed, followed by two additional washing steps as described above. The glass slide was then embedded in Mowiol and covered with glass. Slides were stored at 4° C. and sealed with nail polish after two days.

### Example 6

### Fluorescence Microscopy

### Standard Microscopy

Cell counting and viability assessment were performed on a Nikon TMS F light microscope. Manual cell picking for clonal selection (supra) was done on a Nikon Eclipse TE300 using the corresponding fluorescence filters. Counting the number of cells in specific slide wells that contained tripartite AFT nuclear spots (infra) was performed on a Leica DM IRE2 inverse microscope.

### Confocal Laser Scanning Microscopy (CLSM)

Subcellular protein distribution of fluorescently tagged proteins or proteins stained as described supra was analyzed on a Leica TCS/SP2 confocal microscope. Pictures were acquired at a resolution of 1024×1024 using the 63×H₂O immersion objective. Typically, 5-8 z-axis sections were recorded across the entire height of embedded cells so that typical section separations were 0.5 μm. Settings for the excitation and emission (i.e. detection) wavelengths for different experimental conditions are given below:

TABLE 12
Excitation and photomultiplier settings used for confocal microscopy

| Fluorophore | | Excitation | Detection |
|---|---|---|---|
| of interest | Laser | (nm) | (nm, PMT-window) |
| DAPI DNA stain | UV | 405 | 410-430 |
| CFP | Argon | 458 | 465-485 |
| Citrine | Argon | 514 | 525-580 (wide, no Cy3 |
| | | | in sample) 525-545 (Cy3 |
| | | | present - prevents |
| | | | glow-through) |
| Cy3 | Helium-Neon | 543 | 553-600 |
| Cy5 | Helium-Neon | 633 | 655-710 |

PMT: photomultiplier tube,
CFP: Cyan Fluorescent protein,
Cy3 and Cy5: fluorescent dyes conjugated to secondary antibodies,
UV: ultraviolet light

### Example 7

### APP-TAP-AICD Transgenic Mouse

APP was mutagenized to contain BsrgGI and NcoI restriction sites at K650 and H657, respectively, without changing the aa composition. As the TAP cassette has an internal NcoI restriction site, sticky end cloning (supra) was used to prepare the TAP cassette for entry between these two amino acids, yielding full length APP that contains the TAP tag juxtamembraneously. This construct was cloned by blunt end cloning in front of a PrP promoter. After removal of vector sequence, the linear construct was injected into pronuclei of fertilized zygotes of B6D2F1 mice. Founders were screened for transgene expression by tail PCR and Western blot analysis by 6E10 Aβ-specific antibody, and the line used in this study was expanded by pairing littermates. All MS results shown were derived from hemizygous mice.

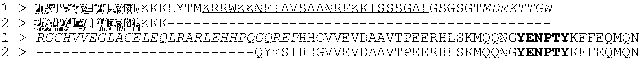

The above alignment shows where the TAP construct was inserted into the normal APP C-terminal sequence. Legend: 1>APP-TAP-AICD (SEQ ID NOs: 7 and 8), 2>APP (SEQ ID NOs: 9 and 10), grey: transmembrane region, underlined: Calmodulin binding peptide, italic: Streptavidin binding peptide.

### Example 8

### Verification of APP-Interacting Molecules

PCR was performed on a human brain-derived cDNA library to amplify the genes of the candidate APP-interacting proteins. Amplified cDNAs were cloned into expression vectors with the in frame addition of HA tags.

Single candidate genes were co-transfected with APP into primary mouse neurons. Clear co-localization in vesicular structures throughout the neurites was seen for SNAP-25, NSF, VAMP2 and Synaptotagmin1. With VAMP2 and Synaptotagmin1 there was a close to 100% overlap of vesicles stained for APP. This clearly shows that APP is localized to synaptic vesicles that contain the SNARE protein VAMP2 required for fusion and the Ca²⁺-sensor Synaptotagmin1.

To further verify the correct identification of APP-interacting proteins by the MS experiments APP-pulldown was performed from transgenic and wild-type mouse brains. Analysis of the isolated proteins showed that full-length APP and cleaved APP-stubs are isolated with Streptavidin purification (L, whole brain lysate; E, eluate after streptavidin purification) only from transgenic brains but not from wild-type mice. In addition, in isolates from transgenic mice we could identify Synapsin1, Syntaxin1, SNAP-25, VAMP2 and Synaptotagmin1 to be strongly enriched in comparison to eluates from wild-type mice.

Furthermore, the expression of the candidate proteins was analyzed in synaptosomes of APP knockout, compared to wild-type animals. No difference in expression levels were detected. Therefore, it is expected that the function of APP in synaptic vesicle cycling can only be determined studying physiological parameters of synaptic vesicle release.

### Reagents

### Chemicals

Standard chemicals were purchased from Sigma. Specialty chemicals and kits, including order numbers, are indicated in the Materials and Methods section.

Buffers

| Buffer | Composition |
|---|---|
| 10 x PBS | 1.4 M NaCl, 27 mM KCl, 100 mM Na₂HPO₄, 18 mM KH₂PO₄ |
| 10 x TBS | 0.84 M Tris-HCl, 0.16 M Tris-Base, 1.5 M NaCl |
| 2D Gel Buffer | 0.375 M Tris-Base, pH 8.8, 5% glycerol and 1% SDS |
| 2D Running Buffer | 25 mM Tris-Base, 0.2 M Glycine, 0.1% SDS |
| 2DGE-equilibration buffer | 2% DTT, 6 M Urea, 2% SDS, 50 mM Tris-Base pH 8.8, 20% glycerol |
| CBB | CXB, 100 mM CaCl₂, Complete Proteinase Inhibitor Cocktail (Roche) 1 |
| | tablet/50 ml, β-ME 10 mM |
| CCMB | Potassium acetate (10 mM), Glycerol (10% (w/v)), CaCl₂ (80 mM), |
| | MnCl₂ (20 mM), MgCl₂ (10 mM), pH 6.0, sterile filtration |
| CEB | CXB, 100 mM EGTA-Na₄ |
| CXB stock | Tris HCl 10 mM, NaCl 150 mM, MgAc (•4H2O) 1 mM, Imidazol 1 mM, |
| | SBB detergent 0.1%, pH 8.0 |
| cytoMgCa buffer | 140 mM KCl, 12 mM NaCl, 5 mM MgCl₂, 2 mM CaCl₂, pH 7.4 |
| EC Resuspension buffer | 50 mM NaPi, 10% glycerol, 300 mM NaCl, 200 mM imidazole, pH 7.0 |
| LB Agar | LB medium, 15 g/l Bacto Agar |
| LB Medium | 10 g/l Bacto Tryptone, 5 g/l Bacto yeast extract, 10 g/l NaCl, pH 7.0 |
| MALDI matrix solution | 2.5 mg/ml CHCA in 70% ACN, 0.1% TFA |
| PreScission buffer | 50 mM TrisHCl, 2.5 mM EDTA, 1 mM DTT, 2 mM NaCl, pH 7.0 |
| reduction & alkylation buffer | 70% H₂O, 10% TCEP, 10% Rapigest (10 mg/ml; 1% in 50 mM |
| | NH₄HCO₃, pH 7.8), 100 mM NH₄HCO₃ solution, pH 7.8 |
| Rehydration Buffer | 8.5 M Urea, 4% CHAPS, 0.5% pharmalytes pH 3-10, 1.2% DeStreak |
| | reagent |
| SBB lysis buffer | NP40 or Triton X100; 0.4%-1%; TrisHCl 40 mM, KCl 150 mM, 5% |
| | glycerol, Complete Proteinase Inhibitor Cocktail (Roche) 1 tablet/50 ml, |
| | β-ME 5 mM, pH = 7.4 |
| SDS sample buffer | 450 mM Tris-HCl, 12% (w/v) glycerol, 4% SDS, 7.5 ‰ Coomassie |
| | blue, 5% □-ME, pH 8.45 |
| SEB | same buffer as SWB + 2 mM Biotin, NH₄HCO₃ instead of TrisHCl |
| ssDevelop | 6% Na₂CO₃, 0.05% formalin, 0.0004% Na₂SO₃ |
| ssFix | 50% MetOH, 12% acetic acid, 0.05% Formalin (35% formaldehyde) |
| ssStain | 0.2% AgNO₃, 0.076% formalin |
| ssStop | 50% MetOH, 12% acetic acid |
| SWB | same buffer as SBB lysis, but without Proteinase inhibitor cocktail, and |
| | only 0.4% of detergent |
| SynaptosomePrep buffer A | 0.32 M sucrose, 10 mM HEPES, 1 mM MgCl₂, 0.5 mM CaCl₂, |
| | Complete Proteinase Inhibitor Cocktail (Roche) 1 tablet/50 ml, pH 7.4 |
| WB transfer buffer | 3 mM Tris-Base, 19.2 mM Glycine, 15% MetOH |

### Additional MS Data

TABLE 13
Comparison of proteins identified by AICD(wt)/(mut) pull-down of SH-SY5Y
cell lysate cytosolic fraction

| Swissprot | AICD(wt) | Swissprot | AICD(mut) |
|---|---|---|---|
| P42704 | 130 kDa leucine-rich protein | | |
| | 27 × 40S ribosomal protein subunits | | 14 × 40S ribosomal protein subunits |
| | 37 × 60S ribosomal protein subunits | | 22 × 60S ribosomal protein subunits |
| P68133 | Actin, alpha skeletal muscle | P10809 | 60 kDa heat shock protein, mitochondrial |
| | | | precursor |
| P63261 | Actin, cytoplasmic 2 | P68133 | Actin, alpha skeletal muscle |
| P12236 | ADP, ATP carrier protein, liver | P60709 | Actin, cytoplasmic 1 |
| | isoform T2 | | |
| P06576 | ATP synthase beta chain, | P12236 | ADP, ATP carrier protein, liver isoform T2 |
| | mitochondrial precursor | | |
| Q96HW2 | ATP5A1 protein | P68104 | Elongation factor 1-alpha 1 |
| P61221 | ATP-binding cassette sub-family E | P49411 | Elongation factor Tu, mitochondrial |
| | member 1 | | precursor |
| P13010 | ATP-dependent DNA helicase II, 80 kDa | P07900 | Heat shock protein HSP 90-alpha |
| | subunit | | |
| O75531 | Barrier-to-autointegration factor | P04792 | Heat-shock protein beta-1 |
| Q9UJS0 | Calcium-binding mitochondrial carrier | Q5T6W5 | Heterogeneous nuclear ribonucleoprotein K |
| | protein Aralar2 | | |
| P06493 | Cell division control protein 2 | P20670 | Histone H2A |
| | homolog | | |
| Q96D46 | CGI-07 protein | Q96BA7 | HNRPU protein |
| Q9Y5B9 | Chromatin-specific transcription | Q9NTK6 | Hypothetical protein DKFZp761K0511 |
| | elongation factor FACT 140 kDa | | |
| | subunit | | |
| P53621 | Coatomer alpha subunit | Q8N1K5 | Hypothetical protein FLJ40584 |
| Q99829 | Copine I | Q9HBR7 | Hypothetical protein |
| P12532 | Creatine kinase, ubiquitous | P35527 | Keratin, type I cytoskeletal 9 |
| | mitochondrial precursor | | |
| O00571 | DEAD-box protein 3, X-chromosomal | P04264 | Keratin, type II cytoskeletal 1 |
| P78527 | DNA-dependent protein kinase | Q5VLR4 | Lung cancer oncogene 7 |
| | catalytic subunit | | |
| Q14204 | Dynein heavy chain, cytosolic | Q96RQ3 | Methylcrotonoyl-CoA carboxylase alpha |
| | | | chain, mitochondrial precursor |
| Q6IQ15 | EEF1A1 protein | Q9HCC0 | Methylcrotonoyl-CoA carboxylase beta |
| | | | chain, mitochondrial precursor |
| P13639 | Elongation factor 2 | Q9H3F4 | MSTP030 |
| P49411 | Elongation factor Tu, mitochondrial | P35579 | Myosin heavy chain, nonmuscle type A |
| | precursor | | |
| P60842 | Eukaryotic initiation factor 4A-I | P35580 | Myosin heavy chain, nonmuscle type B |
| P62495 | Eukaryotic peptide chain release factor | O94832 | Myosin Id |
| | subunit 1 | | |
| P56537 | Eukaryotic translation initiation factor 6 | P60660 | Myosin light polypeptide 6 |
| P06396 | Gelsolin precursor | P19105 | Myosin regulatory light chain 2, |
| | | | nonsarcomeric |
| P00367 | Glutamate dehydrogenase 1, | P67809 | Nuclease sensitive element binding protein 1 |
| | mitochondrial precursor | | |
| P04406 | Glyceraldehyde-3-phosphate | Q5T1D1 | OTTHUMP00000017090 |
| | dehydrogenase, liver | | |
| P07900 | Heat shock protein HSP 90-alpha | Q5T6D9 | OTTHUMP00000039257 |
| P04792 | Heat-shock protein beta-1 | Q9P2W0 | PEG8\IGF2AS protein |
| Q5T6W5 | Heterogeneous nuclear | Q8NC51 | Plasminogen activator inhibitor 1 RNA- |
| | ribonucleoprotein K, | | binding protein |
| Q00839 | Heterogenous nuclear | P43490 | Pre-B cell enhancing factor precursor |
| | ribonucleoprotein U | | |
| Q92522 | Histone H1x | O00425 | Putative RNA binding protein KOC |
| Q6IBM4 | HNRPH1 protein | P11498 | Pyruvate carboxylase, mitochondrial |
| | | | precursor |
| Q8TCJ8 | Hypothetical protein DKFZp564C172, | Q6NZ55 | ribosomal protein L13 |
| Q7Z349 | Hypothetical protein | Q8N6Z7 | ribosomal protein S6 |
| | DKFZp686M22160 | | |
| Q9NTK6 | Hypothetical protein | Q8TBK5 | RPL6 protein |
| | DKFZp761K0511 | | |
| Q6ZS99 | Hypothetical protein FLJ45706 | P02768 | Serum albumin precursor |
| Q7L7R3 | Interleukin enhancer binding factor 2, | Q9UNL2 | Translocon-associated protein, gamma |
| | 45 kDa | | subunit |
| Q12906 | Interleukin enhancer-binding factor 3 | Q15657 | Tropomyosin isoform |
| P42167 | Lamina-associated polypeptide 2, | Q71U36 | Tubulin alpha-3 chain |
| | isoforms beta\gamma | | |
| Q5VLR4 | Lung cancer oncogene 7 | P68363 | Tubulin alpha-ubiquitous chain |
| Q9HCC0 | Methylcrotonoyl-CoA carboxylase | P68371 | Tubulin beta-? chain |
| | beta chain, mitochondrial precursor | | |
| Q9BRJ6 | MGC11257 protein | P07437 | Tubulin beta-2 chain |
| Q8TBR1 | MGC27348 protein | P08670 | Vimentin |
| Q9H3F4 | MSTP030 | Q9UDW8 | WUGSC: H_DJ0747G18.3 protein |
| Q9H3E4 | MSTP041 | | |
| Q9P0V5 | MYB-binding protein 1A | | |
| Q6IBG5 | MYL6 protein | | |
| P35579 | Myosin heavy chain, nonmuscle type A | | |
| P35580 | Myosin heavy chain, nonmuscle type B | | |
| O43795 | Myosin Ib | | |
| P14649 | Myosin light chain 1, slow-twitch muscle A | | |
| | isoform | | |
| P60660 | Myosin light polypeptide 6 | | |
| P19105 | Myosin regulatory light chain 2, | | |
| | nonsarcomeric | | |
| Q9BQC5 | NONO protein | | |
| P67809 | Nuclease sensitive element binding | | |
| | protein 1 | | |
| Q6V962 | Nucleophosmin | | |
| Q5T1D1 | OTTHUMP00000017090 | | |
| Q8NC51 | Plasminogen activator inhibitor 1 | | |
| | RNA-binding protein | | |
| P09874 | Poly [ADP-ribose] polymerase-1 | | |
| P43490 | Pre-B cell enhancing factor precursor | | |
| P17844 | Probable RNA-dependent helicase p68 | | |
| Q9UQ80 | Proliferation-associated protein 2G4 | | |
| O14547 | PRP8 protein | | |
| O00425 | Putative RNA binding protein KOC | | |
| P32322 | Pyrroline-5-carboxylate reductase | | |
| Q96C36 | Pyrroline-5-carboxylate reductase | | |
| Q6NZ55 | ribosomal protein L13 | | |
| Q8N5Z7 | ribosomal protein L6 | | |
| Q8NI61 | ribosomal protein S2 | | |
| Q8N6Z7 | ribosomal protein S6 | | |
| Q9BSW5 | RPS2 protein | | |
| Q8WVC2 | RPS21 protein | | |
| Q7Z7N7 | SLC25A3 protein | | |
| Q15393 | Splicing factor 3B subunit 3 | | |
| Q01081 | Splicing factor U2AF 35 kDa subunit | | |
| P26368 | Splicing factor U2AF 65 kDa subunit | | |
| Q08945 | Structure-specific recognition protein 1 | | |
| P30048 | Thioredoxin-dependent peroxide reductase, | | |
| | mitochondrial precursor | | |
| P20290 | Transcription factor BTF3 | | |
| P09493 | Tropomyosin 1 alpha chain | | |
| P67936 | Tropomyosin alpha 4 chain | | |
| Q15657 | Tropomyosin isoform | | |
| Q71U36 | Tubulin alpha-3 chain | | |
| P68371 | Tubulin beta-? chain | | |
| P07437 | Tubulin beta-2 chain | | |
| Q13509 | Tubulin beta-3 chain | | |
| Q969E5 | Tubulin, beta 4 | | |
| Q9BVA1 | Tubulin, beta polypeptide paralog | | |
| O75643 | U5 small nuclear ribonucleoprotein | | |
| | 200 kDa helicase | | |
| Q5RKT7 | Ubiquitin and ribosomal protein S27a | | |
| P08670 | Vimentin | | |
| Q9UDW8 | WUGSC: H_DJ0747G18.3 protein | | |

For each analysis, four 15 cm plates of confluent SH-SY5Y cells were lysed, bound to AICD(wt)/(mut) resins, eluted in either 0.5 M, 1 M, 2 M or 4 M GuHCl, dialyzed into Trypsin compatible buffer, trypsinized, Zip-Tip desalted and analyzed on a ThermoFinnigan Deca ion trap after RP separation. The four individual fractions from each sample were analyzed separately and pooled afterwards for analysis. The CID spectra were searched against a human Swissprot/Genbank combined database using the transproteomic pipeline. Proteins identified in both samples are given italic; those unique to one sample are given without specific indications.

TABLE 14
PreScission Protease protocol results in strong reduction of contaminant
proteins and first physiologically relevant IDs

| IPI ID | PrSciAICD(wt) | IPI ID | PrSciAICD(mut) |
|---|---|---|---|
| IPI00221093 | 40s ribosomal protein s17. | IPI00180776 | 29 kda protein. |
| IPI00021428 | Actin, alpha skeletal muscle. | IPI00383237 | 58 kda protein. |
| IPI00021439 | Actin, cytoplasmic 1. | IPI00003362 | 78 kda glucose-regulated protein |
| | | | precursor. |
| IPI00022434 | Alb protein. | IPI00021428 | Actin, alpha skeletal muscle. |
| IPI00465248 | Alpha-Enolase. | IPI00021439 | Actin, cytoplasmic 1. |
| IPI00303476 | ATP Synthase beta chain, | IPI00022434 | Alb protein. |
| | mitochondrial precursor. | | |
| IPI00020599 | Calreticulin precursor. | IPI00465248 | Alpha-Enolase. |
| IPI00442122 | CDna16459, clone brcan2002473, | IPI00440493 | Atp synthase alpha chain, |
| | moderately similar to Tropomyosin, | | mitochondrial precursor. |
| | fibroblast isoform 2. | | |
| IPI00014230 | Complement component 1, q | IPI00220834 | ATP-dependent DNA helicase 2 |
| | subcomponent-binding protein, | | subunit 2. |
| | mitochondrial precursor. | | |
| IPI00465439 | Fructose-bisphosphate Aldolase a. | IPI00020599 | Calreticulin precursor. |
| IPI00219219 | Galectin-1. | IPI00382990 | Derp12. |
| IPI00386208 | Gastric-associated differentially- | IPI00027230 | Endoplasmin precursor. |
| | expressed protein ya61p. | | |
| IPI00219018 | Glyceraldehyde-3-phosphate | IPI00163187 | Fascin. |
| | dehydrogenase. | | |
| IPI00442866 | Hypothetical protein flj26480. | IPI00219219 | Galectin-1. |
| IPI00431701 | Hypothetical protein. | IPI00386208 | Gastric-associated differentially- |
| | | | expressed protein ya61p. |
| IPI00163286 | Loh12cr1. | IPI00219018 | Glyceraldehyde-3-phosphate |
| | | | Dehydrogenase. |
| IPI00329351 | P60, 60-kda heat shock protein, | IPI00382470 | Heat shock protein HSP 90-alpha 2. |
| | hsp60 (fragment). | | |
| IPI00102950 | Predicted: hypothetical protein | IPI00442866 | Hypothetical protein flj26480. |
| | loc170082 isoform 1. | | |
| IPI00550882 | Pyrroline-5-carboxylate Reductase 1. | IPI00163286 | Loh12cr1. |
| IPI00470610 | Pyrroline-5-carboxylate Reductase 2. | IPI00329351 | P60, 60-kda heat shock protein, hsp60 |
| | | | (fragment). |
| IPI00299402 | Pyruvate Carboxylase, mitochondrial | IPI00179964 | Splice isoform 1 of polypyrimidine |
| | precursor. | | tract-binding protein 1. |
| IPI00024067 | Similar to Clathrin heavy chain. | IPI00550363 | Transgelin-2. |
| IPI00003865 | Splice isoform 1 of heat shock | IPI00465028 | Triosephosphate Isomerase 1 variant |
| | cognate 71 kda protein. | | (fragment). |
| IPI00009960 | Splice isoform 1 of mitochondrial | IPI00216134 | Tropomyosin 1 alpha chain isoform 7. |
| | inner membrane protein. | | |
| IPI00333771 | Splice isoform 5 of Caldesmon. | IPI00387144 | Tubulin alpha-ubiquitous chain. |
| IPI00217563 | Splice isoform beta-1a of integrin | IPI00011654 | Tubulin beta-2 chain. |
| | beta-1 precursor. | | |
| IPI00412681 | Splice isoform 1-APP752 of Amyloid | IPI00216308 | Voltage-dependent anion-selective |
| | beta a4 Protein Precursor (fragment). | | channel protein 1. |
| IPI00216393 | Splice isoform non-brain of | IPI00219757 | 2 Glutathione s-Transferase p. |
| | Clathrin light chain a. | | |
| IPI00180675 | Tubulin alpha-3 chain. | | |
| IPI00011654 | Tubulin beta-2 chain. | | |
| IPI00456429 | Ubiquitin and ribosomal protein 140 | | |
| | precursor. | | |
| IPI00216308 | Voltage-dependent anion-selective | | |
| | channel protein 1. | | |

Lysates from undifferentiated SH-SY5Y cells were purified using PrSciAICD(wt)/(mut) peptides and specifically eluted as already described supra. Proteins identified in both samples are given italic; those unique to one sample are given without specific indications. Bait peptide is shaded blue and putatively interesting proteins are given in bold.

### REFERENCES

Abbenante, G., et al. (2000). “Inhibitors of beta-amyloid formation based on the beta-secretase cleavage site.” Biochem Biophys Res Commun 268(1): 133-5.
Ando, K., et al. (2001). “Phosphorylation-dependent regulation of the interaction of amyloid precursor protein with Fe65 affects the production of beta-amyloid.” J Biol Chem 276(43): 40353-61.
Annaert, W. G., et al. (1999). “Presenilin 1 controls gamma-secretase processing of amyloid precursor protein in pre-golgi compartments of hippocampal neurons.” J Cell Biol 147(2): 277-94.
Asada, T., et al. (1996). “Prevalence of dementia and distribution of ApoE alleles in Japanese centenarians: an almost-complete survey in Yamanashi Prefecture, Japan.” J Am Geriatr Soc 44(2): 151-5.
Bachman, D. L., et al. (1992). “Prevalence of dementia and probable senile dementia of the Alzheimer type in the Framingham Study.” Neurology 42(1): 115-9.
Baek, S. H., et al. (2002). “Exchange of N-CoR corepressor and Tip60 coactivator complexes links gene expression by NF-kappaB and beta-amyloid precursor protein.” Cell 110(1): 55-67.
Bales, K. R., et al. (1997). “Lack of apolipoprotein E dramatically reduces amyloid beta-peptide deposition.” Nat Genet. 17(3): 263-4.
Bauer, J., et al. (1991). “Interleukin-6 and alpha-2-macroglobulin indicate an acute-phase state in Alzheimer's disease cortices.” FEBS Lett 285(1): 111-4.
Behl, C., J. B. Davis, R. Lesley and D. Schubert (1994). “Hydrogen peroxide mediates amyloid beta protein toxicity.” Cell 77(6): 817-27.
Biederer, T., X. Cao, T. C. Sudhof and X. Liu (2002). “Regulation of APP-dependent transcription complexes by Mint/X11s: differential functions of Mint isoforms.” J Neurosci 22(17): 7340-51.
Borg, J. P., J. Ooi, E. Levy and B. Margolis (1996). “The phosphotyrosine interaction domains of X11 and FE65 bind to distinct sites on the YENPTY motif of amyloid precursor protein.” Mol Cell Biol 16(11): 6229-41.
Borg, J. P., et al. (1998). “The X11 alpha protein slows cellular amyloid precursor protein processing and reduces Abeta40 and Abeta42 secretion.” J Biol Chem 273(24): 14761-6.
Bouwmeester, T., et al. (2004). “A physical and functional map of the human TNF-alpha/NF-kappa B signal transduction pathway.” Nat Cell Biol 6(2): 97-105.
Braak, H. and E. Braak (1995). “Staging of Alzheimer's disease-related neurofibrillary changes.” Neurobiol Aging 16(3): 271-8; discussion 278-84.
Bressler, S. L., et al. (1996). “cDNA cloning and chromosome mapping of the human Fe65 gene: interaction of the conserved cytoplasmic domains of the human beta-amyloid precursor protein and its homologues with the mouse Fe65 protein.” Hum Mol Genet. 5(10): 1589-98.
Buxbaum, J. D., E. H. Koo and P. Greengard (1993). “Protein phosphorylation inhibits production of Alzheimer amyloid beta/A4 peptide.” Proc Natl Acad Sci USA 90(19): 9195-8.
Buxbaum, J. D., et al. (1998). “Evidence that tumor necrosis factor alpha converting enzyme is involved in regulated alpha-secretase cleavage of the Alzheimer amyloid protein precursor.” J Biol Chem 273(43): 27765-7.
Cai, H., et al. (2001). “BACE1 is the major beta-secretase for generation of Abeta peptides by neurons.” Nat Neurosci 4(3): 233-4.
Cao, X. and T. C. Sudhof (2001). “A transcriptionally [correction of transcriptively] active complex of APP with Fe65 and histone acetyltransferase Tip60.” Science 293(5527): 115-20.
Cao, X. and T. C. Sudhof (2004). “Dissection of amyloid-beta precursor protein-dependent transcriptional transactivation.” J Biol Chem 279(23): 24601-11.
Capell, A., et al. (1998). “The proteolytic fragments of the Alzheimer's disease-associated presenilin-1 form heterodimers and occur as a 100-150-kDa molecular mass complex.” J Biol Chem 273(6): 3205-11.
Carey, R. M., B. A. Balcz, I. Lopez-Coviella and B. E. Slack (2005). “Inhibition of dynamin-dependent endocytosis increases shedding of the amyloid precursor protein ectodomain and reduces generation of amyloid beta protein.” BMC Cell Biol 6: 30.
Chen, W. J., J. L. Goldstein and M. S. Brown (1990). “NPXY, a sequence often found in cytoplasmic tails, is required for coated pit-mediated internalization of the low density lipoprotein receptor.” J Biol Chem 265(6): 3116-23.
Chen, X., et al. (2002). “Three-dimensional structure of the complexin/SNARE complex.” Neuron 33(3): 397-409.
Citron, M., D. B. Teplow and D. J. Selkoe (1995). “Generation of amyloid beta protein from its precursor is sequence specific.” Neuron 14(3): 661-70.
Cotman, C. W. and A. J. Anderson (1995). “A potential role for apoptosis in neurodegeneration and Alzheimer's disease.” Mol Neurobiol 10(1): 19-45.
Cottrell, B. A., et al. (2005). “A pilot proteomic study of amyloid precursor interactors in Alzheimer's disease.” Ann Neurol 58(2): 277-89.
Coulson, E. J., K. Paliga, K. Beyreuther and C. L. Masters (2000). “What the evolution of the amyloid protein precursor supergene family tells us about its function.” Neurochem Int 36(3): 175-84.
Cummings, B. J., C. J. Pike, R. Shankle and C. W. Cotman (1996). “Beta-amyloid deposition and other measures of neuropathology predict cognitive status in Alzheimer's disease.” Neurobiol Aging 17(6): 921-33.
De Strooper, B., et al. (1998). “Deficiency of presenilin-1 inhibits the normal cleavage of amyloid precursor protein.” Nature 391(6665): 387-90.
De Strooper, B., L. Umans, F. Van Leuven and H. Van Den Berghe (1993). “Study of the synthesis and secretion of normal and artificial mutants of murine amyloid precursor protein (APP): cleavage of APP occurs in a late compartment of the default secretion pathway.” J Cell Biol 121(2): 295-304.
Delaere, P., et al. (1990). “Large amounts of neocortical beta A4 deposits without neuritic plaques nor tangles in a psychometrically assessed, non-demented person.” Neurosci Lett 116(1-2): 87-93.
Domon, B. and R. Aebersold (2006). “Mass spectrometry and protein analysis.” Science 312(5771): 212-7.
Duyckaerts, C., et al. (1985). “Cortical atrophy in senile dementia of the Alzheimer type is mainly due to a decrease in cortical length.” Acta Neuropathol (Berl) 66(1): 72-4.
Ebinu, J. O. and B. A. Yankner (2002). “A RIP tide in neuronal signal transduction.” Neuron 34(4): 499-502.
Edbauer, D., et al. (2003). “Reconstitution of gamma-secretase activity.” Nat Cell Biol 5(5): 486-8.
Ehrmann, M. and T. Clausen (2004). “Proteolysis as a regulatory mechanism.” Annu Rev Genet. 38: 709-24.
Ermekova, K. S., et al. (1997). “The WW domain of neural protein FE65 interacts with proline-rich motifs in Mena, the mammalian homolog of Drosophila enabled.” J Biol Chem 272(52): 32869-77.
Ferreira, A., A. Caceres and K. S. Kosik (1993). “Intraneuronal compartments of the amyloid precursor protein.” J Neurosci 13(7): 3112-23.
Forde, C. E., et al. (2002). “A rapid method to capture and screen for transcription factors by SELDI mass spectrometry.” Biochem Biophys Res Commun 290(4): 1328-35.
Funamoto, S., et al. (2004). “Truncated carboxyl-terminal fragments of beta-amyloid precursor protein are processed to amyloid beta-proteins 40 and 42.” Biochemistry 43(42): 13532-40.
Geling, A., et al. (2002). “A gamma-secretase inhibitor blocks Notch signaling in vivo and causes a severe neurogenic phenotype in zebrafish.” EMBO Rep 3(7): 688-94.
Gotz, J. (2001). “Tau and transgenic animal models.” Brain Res Brain Res Rev 35(3): 266-86.
Gotz, J., F. Chen, J. van Dorpe and R. M. Nitsch (2001). “Formation of neurofibrillary tangles in P3011 tau transgenic mice induced by Abeta 42 fibrils.” Science 293(5534): 1491-5.
Gretener, D. (2005). “A proteomics approach to the diagnosis of Alzheimer's Disease.” ETH Zürich dissertation (16263).
Grober, E. and C. Kawas (1997). “Learning and retention in preclinical and early Alzheimer's disease.” Psychol Aging 12(1): 183-8.
Grundke-Iqbal, I., et al. (1986). “Abnormal phosphorylation of the microtubule-associated protein tau (tau) in Alzheimer cytoskeletal pathology.” Proc Natl Acad Sci USA 83(13): 4913-7.
Gu, Y., et al. (2001). “Distinct intramembrane cleavage of the beta-amyloid precursor protein family resembling gamma-secretase-like cleavage of Notch.” J Biol Chem 276(38): 35235-8.
Guenette, S. Y., et al. (1999). “hFE65L influences amyloid precursor protein maturation and secretion.” J Neurochem 73(3): 985-93.
Guenette, S. Y., J. Chen, P. D. Jondro and R. E. Tanzi (1996). “Association of a novel human FE65-like protein with the cytoplasmic domain of the beta-amyloid precursor protein.” Proc Natl Acad Sci USA 93(20): 10832-7.
Gygi, S. P., et al. (1999). “Quantitative analysis of complex protein mixtures using isotope-coded affinity tags.” Nat Biotechnol 17(10): 994-9.
Haass, C., A. Y. Hung and D. J. Selkoe (1991). “Processing of beta-amyloid precursor protein in microglia and astrocytes favors an internal localization over constitutive secretion.” J Neurosci 11(12): 3783-93.
Haass, C., E. H. Koo, D. B. Teplow and D. J. Selkoe (1994). “Polarized secretion of beta-amyloid precursor protein and amyloid beta-peptide in MDCK cells.” Proc Natl Acad Sci USA 91(4): 1564-8.
Hanninen, T., et al. (1995). “A follow-up study of age-associated memory impairment: neuropsychological predictors of dementia.” J Am Geriatr Soc 43(9): 1007-15.
Hardy, J. (1997). “Amyloid, the presenilins and Alzheimer's disease.” Trends Neurosci 20(4): 154-9.
Hardy, J. and D. J. Selkoe (2002). “The amyloid hypothesis of Alzheimer's disease: progress and problems on the road to therapeutics.” Science 297(5580): 353-6.
Hardy, J. A. and G. A. Higgins (1992). “Alzheimer's disease: the amyloid cascade hypothesis.” Science 256(5054): 184-5.
Hata, Y., C. A. Slaughter and T. C. Sudhof (1993). “Synaptic vesicle fusion complex contains unc-18 homologue bound to syntaxin.” Nature 366(6453): 347-51.
Heber, S., et al. (2000). “Mice with combined gene knock-outs reveal essential and partially redundant functions of amyloid precursor protein family members.” J Neurosci 20(21): 7951-63.
Herms, J., et al. (2004). “Cortical dysplasia resembling human type 2 lissencephaly in mice lacking all three APP family members.” Embo J 23(20): 4106-15.
Herskovits, J. S., C. C. Burgess, R. A. Obar and R. B. Vallee (1993). “Effects of mutant rat dynamin on endocytosis.” J Cell Biol 122(3): 565-78.
Higuchi, M., et al. (2005). “19F and 1H MRI detection of amyloid beta plaques in vivo.” Nat Neurosci 8(4): 527-33.
Hiller, K., et al. (2004). “PrediSi: prediction of signal peptides and their cleavage positions.” Nucleic Acids Res 32(Web Server issue): W375-9.
Ho, C. S., et al. (2002). “Synergistic effects of Munc18a and X11 proteins on amyloid precursor protein metabolism.” J Biol Chem 277(30): 27021-8.
Hock, C., et al. (2002). “Generation of antibodies specific for beta-amyloid by vaccination of patients with Alzheimer disease.” Nat Med 8(11): 1270-5.
Hsiao, K., et al. (1996). “Correlative memory deficits, Abeta elevation, and amyloid plaques in transgenic mice.” Science 274(5284): 99-102.
Ideker, T., et al. (2001). “Integrated genomic and proteomic analyses of a systematically perturbed metabolic network.” Science 292(5518): 929-34.
Inomata, H., et al. (2003). “A scaffold protein JIP-1b enhances amyloid precursor protein phosphorylation by JNK and its association with kinesin light chain 1.” J Biol Chem 278(25): 22946-55.
Iqbal, K., et al. (2005). “Tau pathology in Alzheimer disease and other tauopathies.” Biochim Biophys Acta 1739(2-3): 198-210.
Kamal, A., et al. (2001). “Kinesin-mediated axonal transport of a membrane compartment containing beta-secretase and presenilin-1 requires APP.” Nature 414(6864): 643-8.
Kang, J., et al. (1987). “The precursor of Alzheimer's disease amyloid A4 protein resembles a cell-surface receptor.” Nature 325(6106): 733-6.
Keller, A., A. I. Nesvizhskii, E. Kolker and R. Aebersold (2002). “Empirical statistical model to estimate the accuracy of peptide identifications made by MS/MS and database search.” Anal Chem 74(20): 5383-92.
Kimberly, W. T., et al. (2000). “The transmembrane aspartates in presenilin 1 and 2 are obligatory for gamma-secretase activity and amyloid beta-protein generation.” J Biol Chem 275(5): 3173-8.
Kimberly, W. T., J. B. Zheng, S. Y. Guenette and D. J. Selkoe (2001). “The intracellular domain of the beta-amyloid precursor protein is stabilized by Fe65 and translocates to the nucleus in a notch-like manner.” J Biol Chem 276(43): 40288-92.
King, G. D., et al. (2003). “X11alpha modulates secretory and endocytic trafficking and metabolism of amyloid precursor protein: mutational analysis of the YENPTY sequence.” Neuroscience 120(1): 143-54.
King, G. D. and R. Scott Turner (2004). “Adaptor protein interactions: modulators of amyloid precursor protein metabolism and Alzheimer's disease risk?” Exp Neurol 185(2): 208-19.
Klyubin, I., et al. (2005). “Amyloid beta protein immunotherapy neutralizes Abeta oligomers that disrupt synaptic plasticity in vivo.” Nat Med 11(5): 556-61.
Knopman, D. S. and S. Ryberg (1989). “A verbal memory test with high predictive accuracy for dementia of the Alzheimer type.” Arch Neurol 46(2): 141-5.
Kohli, B. M., J. K. Eng, R. M. Nitsch and U. Konietzko (2005). “An alternative sampling algorithm for use in liquid chromatography/tandem mass spectrometry experiments.” Rapid Communications in Mass Spectrometry 19(5): 589-596.
Kohli, B. M. and C. Ostermeier (2003). “A Rubredoxin based system for screening of protein expression conditions and on-line monitoring of the purification process.” Protein Expr Purif 28(2): 362-7.
Koo, E. H. and S. L. Squazzo (1994). “Evidence that production and release of amyloid beta-protein involves the endocytic pathway.” J Biol Chem 269(26): 17386-9.
Koyama, S., L. T. Williams and A. Kikuchi (1995). “Characterization of the interaction of Raf-1 with ras p21 or 14-3-3 protein in intact cells.” FEBS Lett 368(2): 321-5.
Kroenke, C. D., et al. (1997). “Solution conformations of a peptide containing the cytoplasmic domain sequence of the beta amyloid precursor protein.” Biochemistry 36(26): 8145-52.
Laemmli, U. K. (1970). “Cleavage of structural proteins during the assembly of the head of bacteriophage T4.” Nature 227(5259): 680-5.
Lai, A., S. S. Sisodia and I. S. Trowbridge (1995). “Characterization of sorting signals in the beta-amyloid precursor protein cytoplasmic domain.” J Biol Chem 270(8): 3565-73.
Lau, K. F., D. M. McLoughlin, C. Standen and C. C. Miller (2000). “X11 alpha and x11 beta interact with presenilin-1 via their PDZ domains.” Mol Cell Neurosci 16(5): 557-65.
Lau, K. F., et al. (2000). “Fe65 and X11 beta co-localize with and compete for binding to the amyloid precursor protein.” Neuroreport 11(16): 3607-10.
Lazarov, O., et al. (2005). “Environmental enrichment reduces Abeta levels and amyloid deposition in transgenic mice.” Cell 120(5): 701-13.
Levitan, D., et al. (2001). “PS1 N- and C-terminal fragments form a complex that functions in APP processing and Notch signaling.” Proc Natl Acad Sci USA 98(21): 12186-90.
Li, L., et al. (1999). “An atomic model for the pleated beta-sheet structure of Abeta amyloid protofilaments.” Biophys J 76(6): 2871-8.
Li, Z. W., et al. (1996). “Generation of mice with a 200-kb amyloid precursor protein gene deletion by Cre recombinase-mediated site-specific recombination in embryonic stem cells.” Proc Natl Acad Sci USA 93(12): 6158-62.
Lin, A. and B. Dibling (2002). “The true face of JNK activation in apoptosis.” Aging Cell 1(2): 112-6.
Loffler, J. and G. Huber (1992). “Beta-amyloid precursor protein isoforms in various rat brain regions and during brain development.” J Neurochem 59(4): 1316-24.
Lowry, O. H., N. J. Rosebrough, A. L. Farr and R. J. Randall (1951). “Protein measurement with the Folin phenol reagent.” J Biol Chem 193(1): 265-75.
Lue, L. F., et al. (1999). “Soluble amyloid beta peptide concentration as a predictor of synaptic change in Alzheimer's disease.” Am J Pathol 155(3): 853-62.
Mann, D. M., P. O. Yates, B. Marcyniuk and C. R. Ravindra (1986). “The topography of plaques and tangles in Down's syndrome patients of different ages.” Neuropathol Appl Neurobiol 12(5): 447-57.
Marambaud, P., et al. (2002). “A presenilin-1/gamma-secretase cleavage releases the E-cadherin intracellular domain and regulates disassembly of adherens junctions.” Embo J 21(8): 1948-56.
Marquez-Sterling, N. R., A. C. Lo, S. S. Sisodia and E. H. Koo (1997). “Trafficking of cell-surface beta-amyloid precursor protein: evidence that a sorting intermediate participates in synaptic vesicle recycling.” J Neurosci 17(1): 140-51.
Matsuda, S., et al. (2001). “c-Jun N-terminal kinase (JNK)-interacting protein-1b/islet-brain-1 scaffolds Alzheimer's amyloid precursor protein with JNK.” J Neurosci 21(17): 6597-607.
Mattson, M. P., et al. (1993). “Evidence for excitoprotective and intraneuronal calcium-regulating roles for secreted forms of the beta-amyloid precursor protein.” Neuron 10(2): 243-54.
Mattson, M. P., et al. (1992). “beta-Amyloid peptides destabilize calcium homeostasis and render human cortical neurons vulnerable to excitotoxicity.” J Neurosci 12(2): 376-89.
May, P., Y. K. Reddy and J. Herz (2002). “Proteolytic processing of low density lipoprotein receptor-related protein mediates regulated release of its intracellular domain.” J Biol Chem 277(21): 18736-43.
McGeer, P. L., et al. (1993). “Microglia in degenerative neurological disease.” Glia 7(1): 84-92.
McKhann, G., et al. (1984). “Clinical diagnosis of Alzheimer's disease: report of the NINCDS-ADRDA Work Group under the auspices of Department of Health and Human Services Task Force on Alzheimer's Disease.” Neurology 34(7): 939-44.
McLoughlin, D. M. and C. C. Miller (1996). “The intracellular cytoplasmic domain of the Alzheimer's disease amyloid precursor protein interacts with phosphotyrosine-binding domain proteins in the yeast two-hybrid system.” FEBS Lett 397(2-3): 197-200.
Najlerahim, A. and D. M. Bowen (1988). “Regional weight loss of the cerebral cortex and some subcortical nuclei in senile dementia of the Alzheimer type.” Acta Neuropathol (Berl) 75(5): 509-12.
Nesvizhskii, A. I., A. Keller, E. Kolker and R. Aebersold (2003). “A statistical model for identifying proteins by tandem mass spectrometry.” Anal Chem 75(17): 4646-58.
Nishimoto, I., et al. (1993). “Alzheimer amyloid protein precursor complexes with brain GTP-binding protein G(o).” Nature 362(6415): 75-9.
Oddo, S., et al. (2003). “Amyloid deposition precedes tangle formation in a triple transgenic model of Alzheimer's disease.” Neurobiol Aging 24(8): 1063-70.
Ohno, M., et al. (2004). “BACE1 deficiency rescues memory deficits and cholinergic dysfunction in a mouse model of Alzheimer's disease.” Neuron 41(1): 27-33.
Ohta, M., et al. (1993). “Immunohistochemical distribution of amyloid precursor protein during normal rat development.” Brain Res Dev Brain Res 75(2): 151-61.
Ong, S. E., et al. (2002). “Stable isotope labeling by amino acids in cell culture, SILAC, as a simple and accurate approach to expression proteomics.” Mol Cell Proteomics 1(5): 376-86.
Pardossi-Piquard, R., et al. (2005). “Presenilin-dependent transcriptional control of the Abeta-degrading enzyme neprilysin by intracellular domains of betaAPP and APLP.” Neuron 46(4): 541-54.
Pedrioli, P. G., et al. (2004). “A common open representation of mass spectrometry data and its application to proteomics research.” Nat Biotechnol 22(11): 1459-1466.
Postina, R., et al. (2004). “A disintegrin-metalloproteinase prevents amyloid plaque formation and hippocampal defects in an Alzheimer disease mouse model.” J Clin Invest 113(10): 1456-64.
Price, D. L. and S. S. Sisodia (1998). “Mutant genes in familial Alzheimer's disease and transgenic models.” Annu Rev Neurosci 21: 479-505.
Puglielli, L., R. E. Tanzi and D. M. Kovacs (2003). “Alzheimer's disease: the cholesterol connection.” Nat Neurosci 6(4): 345-51.
Ravaglia, G., et al. (1999). “Prevalence and severity of dementia among northern Italian centenarians.” Neurology 53(2): 416-8.
Rawson, R. B. (2002). “Regulated intramembrane proteolysis: from the endoplasmic reticulum to the nucleus.” Essays Biochem 38: 155-68.
Ray, W. J., et al. (1999). “Cell surface presenilin-1 participates in the gamma-secretase-like proteolysis of Notch.” J Biol Chem 274(51): 36801-7.
Richards, D. A., C. Guatimosim and W. J. Betz (2000). “Two endocytic recycling routes selectively fill two vesicle pools in frog motor nerve terminals.” Neuron 27(3): 551-9.
Rigaut, G., et al. (1999). “A generic protein purification method for protein complex characterization and proteome exploration.” Nat Biotechnol 17(10): 1030-2.
Ringstad, N., Y. Nemoto and P. De Camilli (1997). “The SH3p4/Sh3p8/SH3p13 protein family: binding partners for synaptojanin and dynamin via a Grb2-like Src homology 3 domain.” Proc Natl Acad Sci USA 94(16): 8569-74.
Riter, L. S., K. M. Gooding, B. D. Hodge and R. K. Julian, Jr. (2006). “Comparison of the Paul ion trap to the linear ion trap for use in global proteomics.” Proteomics 6(6): 1735-40.
Rovelet-Lecrux, A., et al. (2006). “APP locus duplication causes autosomal dominant early-onset Alzheimer disease with cerebral amyloid angiopathy.” Nat Genet. 38(1): 24-6.
Russo, T., et al. (1998). “Fe65 and the protein network centered around the cytosolic domain of the Alzheimer's beta-amyloid precursor protein.” FEBS Lett 434(1-2): 1-7.
Sabo, S. L., A. F. Ikin, J. D. Buxbaum and P. Greengard (2003). “The amyloid precursor protein and its regulatory protein, FE65, in growth cones and synapses in vitro and in vivo.” J Neurosci 23(13): 5407-15.
Scheinfeld, M. H., S. Matsuda and L. D'Adamio (2003). “JNK-interacting protein-1 promotes transcription of A {beta} protein precursor but not A {beta} precursor-like proteins, mechanistically different than Fe65.” Proc Natl Acad Sci USA 31: 31.
Scheinfeld, M. H., et al. (2002). “Jun NH2-terminal kinase (JNK) interacting protein 1 (JIP1) binds the cytoplasmic domain of the Alzheimer's beta-amyloid precursor protein (APP).” J Biol Chem 277(5): 3767-75.
Schenk, D., et al. (1999). “Immunization with amyloid-beta attenuates Alzheimer-disease-like pathology in the PDAPP mouse.” Nature 400(6740): 173-7.
Selkoe, D. J. (1991). “The molecular pathology of Alzheimer's disease.” Neuron 6(4): 487-98.
Seshadri, S., et al. (1997). “Lifetime risk of dementia and Alzheimer's disease. The impact of mortality on risk estimates in the Framingham Study.” Neurology 49(6): 1498-504.
Shimohama, S., et al. (1997). “Differential involvement of synaptic vesicle and presynaptic plasma membrane proteins in Alzheimer's disease.” Biochem Biophys Res Commun 236(2): 239-42.
Singer, S. J. and N. N. Dewji (2006). “Evidence that Perutz's double-beta-stranded subunit structure for beta-amyloids also applies to their channel-forming structures in membranes.” Proc Natl Acad Sci USA 103(5): 1546-50.
Sisodia, S. S. (1992). “Beta-amyloid precursor protein cleavage by a membrane-bound protease.” Proc Natl Acad Sci USA 89(13): 6075-9.
Small, D. H., et al. (1999). “Neurite-outgrowth regulating functions of the amyloid protein precursor of Alzheimer's disease.” J Alzheimers Dis 1(4-5): 275-85.
Smith, R. P., D. A. Higuchi and G. J. Broze, Jr. (1990). “Platelet coagulation factor XIa-inhibitor, a form of Alzheimer amyloid precursor protein.” Science 248(4959): 1126-8.
Sollner, T., et al. (1993). “SNAP receptors implicated in vesicle targeting and fusion.” Nature 362(6418): 318-24.
Sterner, D. E. and S. L. Berger (2000). “Acetylation of histones and transcription-related factors.” Microbiol Mol Biol Rev 64(2): 435-59.
Strittmatter, W. J., et al. (1993). “Apolipoprotein E: high-avidity binding to beta-amyloid and increased frequency of type 4 allele in late-onset familial Alzheimer disease.” Proc Natl Acad Sci USA 90(5): 1977-81.
Studier, F. W. and B. A. Moffatt (1986). “Use of bacteriophage T7 RNA polymerase to direct selective high-level expression of cloned genes.” J Mol Biol 189(1): 113-30.
Sudhof, T. C. (2004). “The synaptic vesicle cycle.” Annu Rev Neurosci 27: 509-47.
Sumioka, A., et al. (2005). “Role of 14-3-3gamma in FE65-dependent gene transactivation mediated by the amyloid beta-protein precursor cytoplasmic fragment.” J Biol Chem 280(51): 42364-74.
Tanzi, R. E., et al. (1987). “Amyloid beta protein gene: cDNA, mRNA distribution, and genetic linkage near the Alzheimer locus.” Science 235(4791): 880-4.
Tarassishin, L., Y. I. Yin, B. Bassit and Y. M. Li (2004). “Processing of Notch and amyloid precursor protein by gamma-secretase is spatially distinct.” Proc Natl Acad Sci USA 101(49): 17050-5.
Taru, H., Y. Kirino and T. Suzuki (2002). “Differential roles of JIP scaffold proteins in the modulation of amyloid precursor protein metabolism.” J Biol Chem 277(30): 27567-74.
Telese, F., et al. (2005). “Transcription regulation by the adaptor protein Fe65 and the nucleosome assembly factor SET.” EMBO Rep 6(1): 77-82.
van Hemert, M. J., H. Y. Steensma and G. P. van Heusden (2001). “14-3-3 proteins: key regulators of cell division, signalling and apoptosis.” Bioessays 23(10): 936-46.
Vassar, R., et al. (1999). “Beta-secretase cleavage of Alzheimer's amyloid precursor protein by the transmembrane aspartic protease BACE.” Science 286(5440): 735-41.
Von Haller, P. D., et al. (2003). “The Application of New Software Tools to Quantitative Protein Profiling Via Isotope-coded Affinity Tag (ICAT) and Tandem Mass Spectrometry: II. Evaluation of Tandem Mass Spectrometry Methodologies for Large-Scale Protein Analysis, and the Application of Statistical Tools for Data Analysis and Interpretation.” Mol Cell Proteomics 2(7): 428-42.
Von Rotz, R. C., et al. (2004). “The APP intracellular domain forms nuclear multiprotein complexes and regulates the transcription of its own precursor.” J Cell Sci 117(Pt 19): 4435-48.
Wasco, W., et al. (1993). “Isolation and characterization of APLP2 encoding a homologue of the Alzheimer's associated amyloid beta protein precursor.” Nat Genet. 5(1): 95-100.
Weggen, S., et al. (2003). “A beta42-lowering nonsteroidal anti-inflammatory drugs preserve intramembrane cleavage of the amyloid precursor protein and ErbB-4 receptor and signaling through the APP intracellular domain.” J Biol. Chem.
Weidemann, A., et al. (1989). “Identification, biogenesis, and localization of precursors of Alzheimer's disease A4 amyloid protein.” Cell 57(1): 115-26.
Widiger, T. A. and D. B. Samuel (2005). “Diagnostic categories or dimensions? A question for the Diagnostic And Statistical Manual Of Mental Disorders—fifth edition.” J Abnorm Psychol 114(4): 494-504.
Winkler, J., L. J. Thal, F. H. Gage and L. J. Fisher (1998). “Cholinergic strategies for Alzheimer's disease.” J Mol Med 76(8): 555-67.
Xing, H., et al. (2000). “14-3-3 proteins block apoptosis and differentially regulate MAPK cascades.” Embo J 19(3): 349-58.
Yan, R., et al. (2001). “The transmembrane domain of the Alzheimer's beta-secretase BACE1) determines its late. Golgi localization and access to beta-amyloid precursor protein (APP) substrate.” J Biol Chem 276(39): 36788-96.
Yan, W., et al. (2004). “System-based proteomic analysis of the interferon response in human liver cells.” Genome Biol 5(8): R54.
Yao, X., et al. (2001). “Proteolytic 180 labeling for comparative proteomics: model studies with two serotypes of adenovirus.” Anal Chem 73(13): 2836-42.
Yates, J. R., 3rd, J. K. Eng, A. L. McCormack and D. Schieltz (1995). “Method to correlate tandem mass spectra of modified peptides to amino acid sequences in the protein database.” Anal Chem 67(8): 1426-36.
Zheng, H., et al. (1995). “beta-Amyloid precursor protein-deficient mice show reactive gliosis and decreased locomotor activity.” Cell 81(4): 525-31.
Zheng, P., J. Eastman, S. Vande Pol and S. W. Pimplikar (1998). “PAT1, a microtubule-interacting protein, recognizes the basolateral sorting signal of amyloid precursor protein.” Proc Natl Acad Sci USA 95(25): 14745-50.

1. A method of identifying or obtaining a molecule interacting with a neurodegenerative, neurological, or neuropsychiatric disorder-associated protein comprising:
(a) providing the neurodegenerative, neurological, or neuropsychiatric disorder-associated protein, or a fragment thereof, containing a tag within a cell or tissue under conditions allowing complex formation;
(b) subjecting a sample of the cell or tissue to at least one purification step; and
(c) isolating the complex purified in step (b).

2. The method of claim 1, wherein the neurodegenerative, neurological, or neuropsychiatric disorder-associated protein is a member of the amyloid precursor protein (APP)/APP-like protein (APLP)-family.

3. The method of claim 1, wherein the protein is APP.

4. The method of claim 1, wherein the tag comprises streptavidine binding peptide (SBP).

5. The method of claim 46, wherein step (d) comprises mass spectroscopy.

6. The method of claim 5, wherein the mass spectroscopy comprises MALDI-TOF/TOF.

7. The method of claim 5, wherein the mass spectroscopy comprises ion trap and Fourier Transformation (LTQ-FT).

8. The method of claim 1, wherein the sample comprises brain homogenate, brain sections, cerebrospinal fluid, or cells of the brain or CNS.

9. The method of claim 1, wherein said cell or tissue is comprised in or derived from a transgenic animal.

10. The method of claim 47, wherein APP or a fragment thereof is recombinantly expressed in the mouse.

11. The method of claim 47, wherein the mouse is the APP-TAP-AICD mouse.

12. The method of claim 1, wherein the purification step (b) essentially consists of affinity purification or through use of streptavidin.

13. (canceled)

14. The method of claim 1, wherein step (c) or (d) immediately follows step (b) without any further substantial purification step.

15. A complex or interacting molecule that interacts with the neurodegenerative, neurological, or neuropsychiatric disorder-associated protein or fragment thereof as defined in claim 1.

16. The complex or interacting molecule of claim 15, wherein said molecule is a protein or peptide.

17. The complex or interacting molecule of claim 16, wherein said protein is selected from the group consisting of proteins given in tables 1, 2, 4, 5, 13, and 14 in the description.

18. The complex or interacting molecule of claim 16, wherein the protein is selected from the group consisting of (P56564) excitatory amino acid transporter (GLAST), (P62962) profilin-1, (P70296) phosphatidylethanolamine-binding protein (PEBP), elongation factor 1-alpha 2 (EF-1-alpha-2), (P99029) peroxiredoxin 5, (P08228) superoxide dismutase [Cu—Zn], (Q8VCR8) myosin light chain kinase 2, skeletal/cardiac muscle (MLCK2), (P63054) brain-specific polypeptide PEP-19, 5 serine/threonine-protein phosphatase 2A 65 kD regulatory subunit A, (Q3UHC2) leucine-rich repeat kinase 1 (LRRK1), synaptosomal-associated protein 25 (SNAP-25), neuronal membrane glycoprotein M6-b (M6b), N-ethylmaleimide sensitive fusion protein (NSF), plasma membrane calcium-transporting ATPase 2 (PMCA2), Ras-related protein Rab-1A (YPT1-related protein), clathrin coat assembly protein AP180, dynamin-1, (Q9R0P9) ubiquitin carboxyl-terminal hydrolase isozyme L1 (UCH-L1), (P6 1264) syntaxin-1B2, (P43006) excitatory amino acid transporter 2 (GLT-1), (P63044) vesicle-associated membrane protein 2 (VAMP-2), (P46096) synaptotagmin-1, (4624 19) SH3-containing GRB2-like protein 1, (P 17742) peptidyl-prolyl cis-trans isomerase A (rotamase), (P05213) tubulin alpha-2 chain (alpha-tubulin 2), or (Q9D6F9) tubulin beta-4 chain, (P35803) proteolipid protein PLPIdm-20, (P62631) elongation factor 1-alpha 2 (EF1A2) and the mitochondrial ATP synthase subunits e.g. the alpha, beta, gamma and epsilon chains (Q03265, P56480, Q91VR2, Q06185, P56385).

19. The method of claim 1, which is a method of identifying or obtaining a drug.

20. The method of claim 19, wherein a test compound or a collection of test compounds is subjected to the cell or tissue or a sample thereof prior, during or after complex formation between APP or a fragment thereof with its interacting molecule.

21. The method of claim 20, wherein the test compound is selected for its capability of modulating the binding of APP or a fragment thereof to its natural interacting molecule and/or modifying the enzymatic activity of the interacting molecule.

22. The method of claim 19, wherein the natural interacting molecule is a protein.

23. The method of claim 19, wherein the method further comprises performing the method without the test compound or a collection of test compounds, and wherein a decrease of complex formation compared to performing the method without the test compound or collection of test compounds is indicative the presence of a putative drug.

24. The method of claim 19, wherein the compound is a peptide, polypeptide, PNA, peptide mimetic, antibody, nucleic acid molecule, aptamer or small organic compound, capable of interfering with the interaction of APP or its fragment with the natural interacting molecule or substantially suppressing the endogenous expression of the gene encoding the interacting molecule.

25. The method of claim 24, wherein the peptide, polypeptide, or peptide mimetic is derived from a protein binding domain or antibody recognizing the natural interacting molecule.

26.-28. (canceled)

29. A composition for treating or diagnosing a neurodegenerative, neurological, or neuropsychiatric disorder comprising the interacting molecule of claim 15; and optionally a pharmaceutically acceptable carrier or means for detection.

30.-31. (canceled)

32. A method of diagnosing a neurodegenerative, neurological, or neuropsychiatric disorder, said method comprising using the complex or interacting molecule of claim 15 or corresponding nucleic acid or protein/antibody based probes as a diagnostic marker and diagnostic means, respectively.

33. A transgenic non-human animal comprising stably integrated into its genome a foreign nucleic acid molecule encoding a protein involved in the onset or development of a neurodegenerative, neurological, or neuropsychiatric disorder, wherein said encoded protein comprises a tag.

34. (canceled)

35. The transgenic non-human animal of claim 33, wherein said disorder is a neurodegenerative disease.

36. (canceled)

37. The transgenic non-human animal of claim 33, which is a rodent.

38. The transgenic non-human animal of claim 37, wherein the rodent is a mouse.

39. The transgenic non-human animal of claim 38, which is the APP-TAP-AICD mouse.

40. A cell or tissue sample derived from the transgenic non-human animal of claim 33.

41. A method of screening for a drug for the treatment of a neurodegenerative, neurological, or neuropsychiatric disorder, or for diagnosing of or research for any of these disorders, said method comprising using the transgenic animal of claim 33.

42. A microarray comprising at least one complex and/or interacting molecule of claim 15 or a corresponding encoding nucleic acid molecule.

43. A kit useful for performing the method of claim 1, said kit comprising an APP or a fragment thereof, containing a tag or a recombinant nucleic acid molecule encoding such APP or fragment, a purification device, a control APP interacting molecule or a recombinant nucleic acid molecule encoding said control molecule, a suitable detection means, spectroscopic devices and/or monitoring systems capable of monitoring complex formation of tagged APP with an interacting molecule.

44. (canceled)

45. A method for treating a neurodegenerative, neurological, or neuropsychiatric disorder in a subject comprising administering to the subject an agent, wherein said agent
(i) binds to a protein selected from the group consisting of the proteins referred to in tables 1, 2, 4, 5, 13, and 14 and the corresponding human orthologs, paralogs, or homologs thereof; or
(ii) binds to APP and is derived from a protein as defined in (i); wherein such binding results in the inhibition of functions or processing patterns that contribute to central nervous system disease.

46. The method of claim 1, further comprising:
(d) identifying the respective interacting molecule of the neurodegenerative, neurological or neuropsychiatric disorder-associated protein.

47. The method of claim 9, wherein the transgenic animal is a mouse.

48. The transgenic animal of claim 33, wherein the foreign nucleic acid molecule is operably linked to expression control sequences allowing transcription and expression of the gene in the brain and/or CNS of the animal.

49. The transgenic animal of claim 35, wherein the disease is Parkinson's disease or Alzheimer's disease.

50. The cell or tissue sample of claim 40, which is derived from the brain or CNS.

51. A method of screening for a drug for the treatment of a neurodegenerative, neurological, or neuropsychiatric disorder, or for diagnosing of or research for any of these disorders, said method comprising using the tissue sample of claim 40.
